(19) United States
(12) Patent Application Publication
(10) Pub. No.: US 2026/0137810 A1
(43) Pub. Date: May 21, 2026

(54) GENE THERAPY VECTORS FOR THE EXPRESSION OF PREPRODYNORPHIN VARIANTS FOR THE TREATMENT OF EPILEPSY

(71) Applicant: CHARITÉ - UNIVERSITÄTSMEDIZIN BERLIN, Berlin (DE)

(72) Inventors: Regine HEILBRONN, Berlin (DE); Christoph SCHWARZER, Innsbruck (AT); Daniela HÜSER, Berlin (DE)

(73) Assignee: CHARITÉ - UNIVERSITÄTSMEDIZIN BERLIN, Berlin (DE)

(21) Appl. No.: 19/113,786

(22) PCT Filed: Oct. 2, 2023
(86) PCT No.: PCT/EP2023/077251

§ 371 Date: Mar. 20, 2025

(30) Foreign Application Priority Data
Sep. 30, 2022 (EP) 22199230.8
May 12, 2023 (EP) 23173162.1

(51) Int. Cl. A61K 48/00; A61K 38/33; C12N 15/86

(52) U.S. Cl. CPC A61K 48/0058; A61K 38/33; C12N 15/86; C12N 2750/14122; C12N 2750/14143

(57) ABSTRACT

A delivery vector including a DNA sequence encoding pre-prodynorphin or pre-prodynorphin-variants, which drives expression of a pre-propeptide in a target cell, and enables the release of dynorphin or dynorphin-variants from the target cell on demand, and contains a signal peptide, which is a N-terminal extension of nascent polypeptide chains and mediates protein targeting to the lumen of the endoplasmatic reticulum, and contains a N-terminal pro-peptide fragment on the C-terminal end and contains i) a sorting motif including the elements DL and EXyL, or ii) a sorting motif of a pre-pro-neuropeptide or protein sorted to large dense core vesicles, other than pre-prodynorphin, where in certain embodiments x is an integer 1 to 20, y is an integer 1 to 10, and each X is independently an amino acid, and wherein the N-terminal pro-peptide fragment consists of 16 to 90 amino acids, and corresponding peptides, DNA molecules viral vectors and particles.

The present invention provides delivery vectors for transferring a nucleic acid sequence that encodes a pre-propeptide to a cell in vitro, ex vivo or in vivo. The present invention provides methods of delivering a nucleic acid sequence to a cell and methods of treating focal epilepsies.

With a prevalence of 1-2%, epilepsies belong to the most frequent neurological diseases worldwide (McNamara et al., 1999) with focal epilepsy accounting for around 70% of cases. Of these, mesial temporal lobe epilepsy (mTLE) is the most frequent clinical presentation. In mTLE the focus lies in or near the hippocampus where learning, memory and emotional control are modulated. mTLE represents an acquired disease frequently induced by traumatic brain injury. Also, CNS infections, high fevers, brain tumours, or vascular malformations can lead to epileptic foci. With ongoing disease, hippocampal sclerosis with accompanying neurological deficits may develop. These represent key clinical features of this subtype of mTLE (for review, see Engel et al., 2001). Despite the introduction of a large variety of antiepileptic drugs over the last few decades, the rate of drug-resistant epilepsies (30% to 70%) has not improved since the early study of Coatsworth in 1971 (Coatsworth et al., 1971, Loscher et al., 2011). To date, surgical resection of the epileptogenic focus remains as the ultimate treatment option for patients in whom the epileptic focus can be clearly defined and can be separated well from other critical CNS regions. The speech centre near to the hippocampal focus represents a major contraindication for epilepsy surgery. But even if epilepsy surgery can be performed, there is no guarantee for lasting seizure freedom. Only up to 50% of patients remain seizure-free for at least one year after removal of the epileptic focus (Spencer et al., 2008).

Since the early 1980s, there has been evidence that opioids, namely dynorphins (Dyn), act as modulators of neuronal excitability in vitro (Henriksen et al., 1982, Siggins et al., 1986). In line with this, the deletion of the prodynorphin (pDyn) coding sequence in mice (Loacker et al., 2007) and the finding of low Dyn levels in humans due to mutations in the promoter region of the pDyn gene (Stogmann et al., 2002, Gambardella et al., 2003) are associated with increased vulnerability to the development of epilepsy. In most animal models of temporal lobe epilepsy (TLE; comprising epilepsies arising in the temporal lobe=lateral TLE and mTLE), cortical and hippocampal pDyn gene expression is reduced after an initial, short peak of over-expression (for review, see (Simonato et al., 1996, Schwarzer et al., 2009). This finding is in line with the description of assumedly short-lived, post-ictally increased pDyn mRNA levels in hippocampal granule cells (Pirker et al., 2009). In an earlier study, an overall reduction of Dyn-immunoreactivity in surgically removed brain tissue obtained from mTLE patients had been described (de Lanerolle et al., 1997).

Dynorphins act preferentially on kappa opioid receptors (KOR). Despite the reduction of endogenous Dyn, KOR remains available as drug target under epileptic conditions, and the application of KOR agonistic drugs can suppress experimental seizures (Tortella et al., 1988, Takahashi et al., 1990, Solbrig et al., 2006, Loacker et al., 2007, Zangrandi et al. 2016). Various selective KOR agonists applied through different routes of administration yielded time- and dose-dependent effects similar to those upon treatment with phenytoin or phenobarbital in models of epilepsy (for review, see (Simonato et al., 1996). We previously demonstrated that activation of KOR promotes the survival of hippocampal neurons subsequent to the acute epileptic phase after unilateral injection of kainic acid in mice (Schunk et al., 2011).

The object of the present invention is to provide delivery vectors to transfer a nucleic acid sequence encoding a pre-propeptide or a peptide comprising a sequence that enables the packing of said pro-peptide, e.g. a prodynorphin into vesicles, wherein the pro-peptide undergoes maturation and the active substance which is a dynorphin or variant of dynorphin is released upon a series of action potentials that exceed a certain excitation threshold. The object of the present invention is, thus, to provide delivery vectors for transferring a nucleic acid sequence to a cell in vitro, ex vivo or in vivo. Object of the invention is in particular a vector-based therapy for treatment of focal epilepsies with pre-prodynorphin or dynorphin or variants thereof. The inventive delivery vectors comprising a nucleic acid encoding pre-prodynorphin or prodynorphin or dynorphin or variants thereof shall transduce neurons, express, process, store and release pre-prodynorphin or prodynorphin or dynorphin or variants thereof and thus provide activation of KOR in the epileptogenic focus, thereby inhibiting seizures.

It is an aim of the invention to further develop the AAV-based gene vector for the expression of pre-prodynorphin for the therapy of focal epilepsy. Here, AAV vectors are preferably used for the expression of pre-prodynorphin. In the present invention, following vector transduction of neurons, pre-prodynorphin is expressed and processed to dynorphin peptides (dyn) of different types and lengths, which are released after stimulation by the trigger, which is high-frequency excitation.

The present invention is further based on the truncation of the ppDYN protein. In order to avoid interfering with the correct vesicular targeting in neurons or with the correct processing into defined peptides, two strategies were used:

First, the region of the so-called N-peptide of ppDyn was shortened. Here, it was found that this region contains a specific sorting motif which was maintained. Secondly, for some neuropeptides (e.g. pPOMC or ppEnk), these N-terminally located signal and sorting sequences are significantly shorter than the N-terminally located signal and sorting sequence of ppDyn. Therefore, fusion proteins that combine the N-terminal of part of such neuropeptide-precursor molecules stemming from pPOMC with the C-terminal sequence of ppDyn coding for the various active Dyn peptides were constructed (FIG. 1).

### DETAILED DESCRIPTION OF THE INVENTION

Subject matter of the present invention are delivery vectors comprising a DNA sequence encoding pre-prodynorphin or pre-prodynorphin-variants and
wherein said delivery vector drives expression of a pre-propeptide in a target cell, and
wherein said delivery vector comprising said DNA sequence enables the release of dynorphin or dynorphin-variants from the target cell on demand, and
wherein said pre-propeptide is pre-prodynorphin or a pre-prodynorphin-variant and
wherein said pre-propeptide comprises a signal peptide, wherein the signal peptide is a N-terminal extension of a nascent polypeptide chain and wherein said signal peptide mediates protein targeting to the lumen of the endoplasmatic reticulum, and,
wherein said pre-propeptide comprises either (i) a N-terminal pro-peptide fragment on the C-terminal end of said signal peptide and wherein said N-terminal pro-peptide fragment comprises a sorting motif comprising the elements DL and EX_{y}L, in particular a sorting motif consisting of the amino acid sequence DLXₓEX_{y}L (SEQ ID No. 36), where x is an integer from 1 to 20, y is an integer from 1 to 10, and each instance of X may independently be any amino acid (for the avoidance of doubt, this means that in the present invention in the first sequence of e.g. 1 to 20 X, each X may individually be any amino acid, and in the second sequence of e.g. 1 to 10 X, each X may individually be any amino acid, particularly as further defined herein), or wherein said pre-propeptide comprises (ii) a N-terminal pro-peptide fragment of a pre-pro-neuropeptide or protein sorted to large dense core vesicles, other than pre-prodynorphin, on the C-terminal end of said signal peptide and wherein said N-terminal pro-peptide fragment comprises a sorting motif of said pre-pro-neuropeptide or protein sorted to large dense core vesicles, and wherein said N-terminal pro-peptide fragment consists of 16 to 90 amino acids, and
wherein said pre-prodynorphin or pre-prodynorphin-variants comprise at least one of the following sequences selected from the group consisting of a,b,c,d,e,f:
a. Dyn A that is SEQ ID No. 2 (AA 207-223 of SEQ ID No. 1; ppDyn) or a variant thereof consisting of the first 13 AA (first from the N-terminal end) or a variant thereof consisting of the first 8 AA (first from the N-terminal end)
b. Dyn B that is SEQ ID No. 3 (AA 226-238 of SEQ ID No. 1; ppDyn)
c. leumorphin that is SEQ ID No. 4 (AA 226-254 of SEQ ID No. 1; ppDyn)
d. variants of Dyn A according to SEQ ID No. 2 having an amino acid sequence identity of at least 60% within the first 8 AA from the N-terminal end of SEQ ID No. 2 (YGGFLRRI) i.e. having an amino acid sequence identity of at least 60% within the sequence YGGFLRRI comprised in SEQ ID No. 2.
e. variants of Dyn B according to SEQ ID No. 3 having an amino acid sequence identity of at least 60% within the first 8 AA from the N-terminal end of SEQ ID No. 3 (YGGFLRRQ) i.e. having an amino acid sequence identity of at least 60% within the sequence YGGFLRRQ comprised in SEQ ID No. 3.
f. variants of leumorphin according to SEQ ID No. 4 having an amino acid sequence identity of at least 60% within the first 8 AA from the N-terminal end of SEQ ID No. 4 (YGGFLRRQ), i.e. having an amino acid sequence identity of at least 60% within the sequence YGGFLRRQ comprised in SEQ ID No. 4.

In this context, 60% sequence identity is defined as follows: 3 of the first 8 N-terminal amino acids may be removed or replaced by another amino acid. Percentage of sequence identity is calculated for the shortened peptide in case of truncated peptide variants. Introduction of additional amino acids are handled as gap in the original sequence, deletions are handled as gap in the modified peptide for calculation of sequence identity (YGGFLRRQ differs from YG-FLRRQ only by 1 AA, although now AA in positions 3, 4, 5, 6 and 7 are different). In any case a variant of SEQ ID No. 2 having an amino acid sequence identity of at least 60% from the N-terminal end in the first 8 AA may be a variant that comprises the sequence: YGZFLRKZ with each Z individually standing for any amino acid and K resubstituting R in position 7, conserving the peptidase recognition site (RK or RR).

In the present invention, “amino acids” stands for naturally occurring amino acids, which are more particularly the canonical amino acids, i.e. the amino acids that are encoded directly by the codons of the universal genetic code.

Throughout the present invention “Z” in an amino acid sequence stands for any of the naturally occurring amino acids, in a specific embodiment “Z” may be selected from the group comprising alanine, glycine, asparagine, glutamine, leucine, serine, valine and isoleucine.

Pre-pro-neuropeptides other than pre-prodynorphin are known to the person skilled in the art and are described e.g., in Zhang et al. Progress in neurobiology 90 (2010) 276-283:

A pre-pro-neuropeptide or protein sorted to large dense core vesicles other than pre-prodynorphin may be selected from the group comprising but not limited to pre-pro-Substance P, pPOMC, pptachykinin A, pptachykinin B, pproopiomelanocortin, ppcholecystokinin, ppchromogranin B, calcitonin gene-related peptide (CGRP), pre-proEnkephalin, pre-proBDNF, pre-proTachykinin, pre-pro-Somatostatin, pre-pro-VIP, pre-pro-CCK, pre-proNociceptin or pre-proNPY.

Pre-pro-neuropeptides or proteins sorted to large dense core vesicles including pre-prodynorphin have a signal peptide at their extreme N-terminus that directs them to translocate into the endoplasmic reticulum (ER) as above described. Neuropeptides are expressed in neurons and secreted in response to physiological or pathological stimuli which means that they are released on-demand. Neuropeptide prohormones exhibit a great diversity of sorting motifs. Some of them have a sorting motif comprising the elements DL and EX_{y}L, in particular a sorting motif consisting of the amino acid sequence DLXₓEX_{y}L (SEQ ID No. 36), where x is an integer from 1 to 20, y is an integer from 1 to 10, and each instance of X may independently be any amino acid as above described. Other pre-pro-neuropeptides, as e.g., ppneuropeptide Y, comprise another sorting motif than DLXₓEX_{y}L (SEQ ID No. 36). In any event sorting shall be comprised in said above-defined N-terminal pro-peptide fragment that consists of 16 to 90 amino acids.

It should be noted that the distance between the elements DL and EX_{y}L of the sorting motif may vary and in particular it is relevant that these elements are present in the respective amino acid sequence; the sorting motif is typically formed by said elements being in spatial proximity to one another. For the avoidance of doubt, the element DL refers to the amino acid sequence asparagine-leucine, and the element EX_{y}L refers to the amino acid sequence glutamic acid-X_{y}-leucine, wherein X_{y} is as defined herein. In particular embodiments, the elements DL and EX_{y}L occur in the amino acid sequence in question in the order DL . . . EX_{y}L, read from N- to C-terminus of the sequence, in other words, DL is located closer to the N-terminal end than EX_{y}L.

In certain embodiments, in the sorting motif consisting of the amino acid sequence DLXₓEX_{y}L (SEQ ID No. 36), x is an integer from 2 to 13, more particularly 5 to 10, more particularly 5 to 8, or alternatively x is 2, 11 or 13. In certain embodiments, in the sorting motif consisting of the amino acid sequence DLXₓEX_{y}L (SEQ ID No. 36), y is an integer from 1 to 5. In certain embodiments, in the sorting motif consisting of the amino acid sequence DLₓEX_{y}L (SEQ ID No. 36), x is an integer from 2 to 13, more particularly 5 to 10, or alternatively x is 2, 5, 8, 10 or 13, and y is an integer from 1 to 5.

In certain specific peptides, x is 2 (POMC), 11 (pDyn) or 13 (BDNF or TAC1).

In certain specific peptides, y is 1-2 (TAC1), 2 (POMC), 2-5 (BDNF) or 3-5 (pDyn).

According to the present invention said delivery vector comprises a DNA sequence encoding the pre-propeptide of dynorphin or dynorphin-variants. This means said vector comprises a DNA sequence encoding a signal peptide fused to the propeptide. As one aspect, the present invention provides delivery vectors for transferring a nucleic acid to a cell, the delivery vector comprising a segment encoding a signal peptide targeting the pre-propeptide to the lumen of endoplasmatic reticulum. The DNA sequence encoding the signal peptide may be a sequence according to SEQ ID No.: 11. In another aspect of the invention said delivery vector comprises a DNA sequence encoding a propeptide fragment. In a particular aspect of the invention said propeptide fragment is a sequence according to SEQ ID No. 5.

The advantage of the present delivery vectors is a release on demand of dynorphins or dynorphin-variants. Particularly, this means that prodynorphin (or a variant of prodynorphin) is packed into vesicles, undergoes maturation and is released on demand upon high frequency stimulation (e.g. stimulation ≥8 Hz) as it occurs at seizure onset. Particularly, a release-on-demand formulation, thus, provides a pre-prodynorphin (or a variant of pre-prodynorphin) that is then packed into vesicles, undergoes maturation and the active substance which is a dynorphin or variant of dynorphin is released upon a frequency of action potentials that exceeds a certain threshold. In other words, release of dynorphin or dynorphin-variants from the target cell “on demand” particularly denominates a “release upon high frequency stimulation” as it occurs at seizure onset and/or “release upon a frequency of action potentials that exceed a certain threshold”. Said certain threshold may be a threshold that is ≥6 Hz, in another embodiment ≥7 Hz in another embodiment ≥8 Hz, in another embodiment ≥9 Hz. This means said release-on-demand is triggered by increased neuronal firing frequency. Said increased neuronal firing frequency may be measured by EEG (electroencephalography) as spike trains, “increased” means a frequency of spikes in a train measured by EEG in said subject that is ≥6 Hz, in another embodiment ≥7 Hz in another embodiment ≥8 Hz, in another embodiment ≥9 Hz.

This means that the present delivery vectors drive expression of pre-propeptides that enable the provision of dynorphin or dynorphin-variants on demand as such delivery vectors first express the pre-propeptides in neurons, where the resulting propeptides are sorted into large dense core vesicles, where they are enzymatically processed and the derived peptides are stored until a sufficiently intense excitation leads to their release, i.e. said release is triggered by increased neuronal firing frequency as explained above. Dyn peptides bind to pre- and/or postsynaptic KOR which activate G-proteins, which, beside others, regulate ion channels to dampen further amplification and spread of neuronal excitation. The translation of the signal peptide of ppDyn is the initial step, guiding ppDyn into the endoplasmatic reticulum, from where prodynorphin is sorted into “large dense core” vesicles (LDV). Using existing mechanisms in neurons, the prodynorphin is enzymatically processed to mature peptides and transported to axon terminals. LDV are stored in the axon terminals and released in a stimulation-dependent manner. High frequency stimulation as explained above, like at the onset of seizures, induce the release, while low-frequency stimulation does not. This creates a release on demand situation. Released Dyn peptides bind to pre- and/or postsynaptic KOR, which activate G-proteins, that, beside others, regulate ion channels to dampen further amplification and spread of neuronal excitation.

In other words, a release on demand composition is a composition that releases the peptide having agonistic effects on human KOR derived from any of the delivery vectors or recombinant virus particles or liposomes or nanoparticles according to the present invention at the onset of seizures in said subject. The onset of seizures. may be characterized by increased neuronal firing frequency that may be measured by EEG (electroencephalography) as spike trains, increased means a frequency of spikes in a train measured by EEG in said subject that is ≥6 Hz, in another embodiment ≥7 Hz in another embodiment ≥8 Hz, in another embodiment ≥9 Hz.

Subject matter of the present invention is a delivery vector comprising a DNA sequence encoding pre-prodynorphin or pre-prodynorphin-variants wherein said N-terminal pro-peptide fragment consists of 16 to 90 amino acids, preferably 20 to 90 amino acids, preferably 30 to 90 amino acids.

Subject matter of the present invention is a delivery vector comprising a DNA sequence encoding pre-prodynorphin or pre-prodynorphin-variants wherein said N-terminal pro-peptide fragment on the C-terminal end of the signal peptide is a modified propeptide fragment of ppDyn wherein the unmodified propeptide fragment of ppDyn is

SEQ ID No. 5:
DCLSRCSLCA VKTQDGPKPI NPLICSLQCQ AALLPSEEWE
RCQSFLSFFT PSTLGLNDKE DLGSKSVGEG PYSELAKLSG
SFLKELEKSK FLPSISTKEN TLSKSLEEKL RGLSDGFREG
AESELMRDAQ LNDGAMETGT LYLAEEDPKE QV

and wherein the modification of said propeptide fragment of SEQ ID No. 5 is a shortening while maintaining the sorting motif that is consisting of the amino acid sequence DLXₓEX_{y}L (SEQ ID No. 36).

Subject matter of the present invention is a delivery vector comprising a DNA sequence encoding pre-prodynorphin or pre-prodynorphin-variants according to the present invention, wherein said N-terminal pro-peptide fragment on the C-terminal end of the signal peptide is a modified propeptide fragment of ppDyn that comprises or consists of

SEQ ID No. 6:
DCLSRCSLCA VKTQDGPKPI NPLICSLQCQ AALLPSEEWE
RCQSFLSFFT PSTLGLNDKE DLGSKSVGEG PYSELAKLSG
SFLRKEQVKR

Subject matter of the present invention is a delivery vector comprising a DNA sequence encoding pre-prodynorphin or pre-prodynorphin-variants wherein said N-terminal pro-peptide fragment on the C-terminal end of the signal peptide is a modified propeptide fragment of ppDyn that comprises or consists of

SEQ ID No: 7
DLGSKSVGEG PYSELAKLSG SFLKELEKSK FLPSISTKEN
TLSKSLEEKL RGLSDGFREG AESELMRDAQ LNDGAMETGT
LYLAEEDPKE QV
or
SEQ ID No 8
DLGSKSVGEG PYSELAKLSG SFLRKEQV
or
SEQ ID No 9
DLGSKSVGEG PYSELAKLRK EQV
or
SEQ ID No. 55
DLGSKSVGEG PYSELRKEQV.

An embodiment of the present invention is a delivery vector comprising a DNA sequence encoding pre-prodynorphin or pre-prodynorphin-variants wherein said N-terminal pro-peptide fragment on the C-terminal end of the signal peptide is a modified pro-peptide fragment of ppDyn that comprises or consists of

SEQ ID No.: 56
DLGSKSVGEG PYSELAKLSG SFLKKEQV

Another embodiment of the present invention is a delivery vector comprising a DNA sequence encoding pre-prodynorphin or pre-prodynorphin-variants wherein said N-terminal pro-peptide fragment on the C-terminal end of the signal peptide is a modified propeptide fragment of ppDyn that comprises or consists of

SEQ ID No.: 57
DLGSKSVGEG PYSELAKL

Another embodiment of the present invention is a delivery vector comprising a DNA sequence encoding pre-prodynorphin or pre-prodynorphin-variants wherein said N-terminal pro-peptide fragment on the C-terminal end of the signal peptide is a modified propeptide fragment of ppDyn that comprises or consists of

SEQ ID No.: 58
DLGSKSVGEG PYSEL

Subject matter of the present invention is a delivery vector comprising a DNA sequence encoding pre-prodynorphin or pre-prodynorphin-variants wherein said N-terminal pro-peptide region on the C-terminal end of the signal peptide is a modified hybrid propeptide fragment of ppDyn wherein the unmodified propeptide fragment of ppDyn is

SEQ ID No. 5
DCLSRCSLCA VKTQDGPKPI NPLICSLQCQ AALLPSEEWE
RCQSFLSFFT PSTLGLNDKE DLGSKSVGEG PYSELAKLSG
SFLKELEKSK FLPSISTKEN TLSKSLEEKL RGLSDGFREG
AESELMRDAQ LNDGAMETGT LYLAEEDPKE QV

and wherein the modification of said propeptide fragment of SEQ ID No. 5 is a replacement of parts of SEQ ID No. 5 with a propeptide of or a fragment of a propeptide of a neuropeptide, wherein the modified propeptide fragment of ppDyn i) comprises at least one sorting motif comprising the elements DL and EX_{y}L, in particular a sorting motif consisting of the amino acid sequence DLXₓEX_{y}L (SEQ ID No. 36) or ii) comprises a sorting motif of said pre-pro-neuropeptide or protein sorted to large dense core vesicles, other than pre-prodynorphin, as defined further herein. For the avoidance of doubt: the skilled person can readily acknowledge that in this specific context obviously the propeptide of or a fragment of a propeptide of a neuropeptide is different from the unmodified propeptide fragment of ppDyn of SEQ ID No. 5, since parts of the sequence is to be replaced.

Subject matter of the present invention is a delivery vector comprising a DNA sequence encoding pre-prodynorphin or pre-prodynorphin-variants wherein the modified propeptide fragment comprises

SEQ ID No. 10:
MPRSCCSRSG ALLLALLLQ ASMEVRGWCL ESSQCQDLTT
ESNLLECIRA CKP.

Subject matter of the present invention is a delivery vector comprising a DNA sequence encoding pre-prodynorphin or pre-prodynorphin-variants wherein the modified propeptide fragment comprises or consists of a propeptide fragment of pre-proEnkephalin, pre-proBDNF, pre-proTachykinin, pre-pro-Somatostatin, pre-pro-VIP, pre-pro-CCK, pre-proNociceptin or pre-proNPY, wherein said propeptide fragment comprises said sorting motif as described above or another sorting motif. For example for ppNPY a different sorting motif was proposed. In particular, subject matter of the present invention is a delivery vector comprising a DNA sequence encoding pre-prodynorphin or pre-prodynorphin-variants wherein the modified propeptide fragment is selected from the group comprising of any of: SEQ ID Nos: 37 to 52.

These DNA sequences may be selected from DNA sequences encoding for a polypeptide from the group comprising:

SEQ ID No. 37: pre-proenkephalin - N-peptide
MARFLTLCTW LLLLGPGLLA TVRAECSQDC ATCSYRLVRP ADINFLACVM ECEGKLPSLK
IWETCKELLQ LSKPELPQDG TSTLRENSKP EESHLLA
SEQ ID No. 38: pre-proenkephalin-pDyn Hybrid:
MARFLTLCTW LLLLGPGLLA TVRAECSQDC ATCSYRLVRP ADINFLACVM ECEGKLPSLK
IWETCKELLQ LSKPELPQDG TSTLRENSKP EESHLLAKRY GGFLRKYPKR SSEVAGEGDG
DSMGHEDLYK RYGGFLRRIR PKLKWDNQKR YGGFLRRQFK VVTRSQEDPN AYSGELFDA
SEQ ID No. 39: pre-pro-NPY - N-peptide
MLGNKRLGLS GLTLALSLLV CLGALAEA
SEQ ID No. 40: pre-pro-NPY-pDyn Hybrid:
MLGNKRLGLS GLTLALSLLV CLGALAEAKR YGGFLRKYPK RSSEVAGEGD
GDSMGHEDLY KRYGGFLRRI RPKLKWDNQK RYGGFLRRQFK VVTRSQEDPN AYSGELFD
SEQ ID No. 41: pre-pro BDNF - N-peptide
MTILFLTMVI SYFGCMKAAP MKEANIRGQG GLAYPGVRTH GTLESVNGPK AGSRGLTSLA
DTFEHVIEEL LDEDHKVRPN EENNKDADLY TSRVMLSSQV PLEPPLLFLL EEYKNYLDAA
NMSM
SEQ ID No. 42: pre-pro BDNF-pDyn Hybrid:
MTILFLTMVI SYFGCMKAAP MKEANIRGQG GLAYPGVRTH GTLESVNGPK AGSRGLTSLA
DTFEHVIEEL LDEDHKVRPN EENNKDADLY TSRVMLSSQV PLEPPLLFLL EEYKNYLDAA
NMSMKRYGGF LRKYPKRSSEV AGEGDGDSMG HEDLYKRYGG FLRRIRPKLK
WDNQKRYGGF LRRQFKVVTR SQEDPNAYSG ELFD
SEQ ID No. 43: pre-pro-Somatostatin - N-peptide
MLSCRLQCALA ALSIVLALGC VTGAPSDPRL RQFLQKSLAA AAGKQELAKY FLAELLSEPN
QTENDALEPE DLSQAAEQDE MRLELQR
SEQ ID No. 44: pre-pro-Somatostatin-pDyn Hybrid:
MLSCRLQCALA ALSIVLALGC VTGAPSDPRL RQFLQKSLAA AAGKQELAKY FLAELLSEPN
QTENDALEPE DLSQAAEQDE MRLELQRKRY GGFLRKYPKR SSEVAGEGDG
DSMGHEDLYK RYGGFLRRIR PKLKWDNQKR YGGFLRRQFK VVTRSQEDPN AYSGELFD
SEQ ID No. 45: pre-pro-Tachykinin A - N- peptide
MKILVALAVF FLVSTQLFAE EIGANDDLNY WSDWYDSDQI KEELPEPFEH LLQRIA
SEQ ID No. 46: pre-pro-Tachykinin A-pDyn Hybrid:
MKILVALAVF FLVSTQLFAE EIGANDDLNY WSDWYDSDQI KEELPEPFEH LLQRIAKRYG
GFLRKYPKRS SEVAGEGDGD SMGHEDLYKR YGGFLRRIRP KLKWDNQKRY
GGFLRRQFKV VTRSQEDPNA YSGELFD
SEQ ID No. 47: pre-pro-VIP - N-peptide
MDTRNKAQLL VLLTLLSVLF SQTSAWPLYR APSALRLGDR IPFEGANEPD QVSLKEDIDM
LQNALAENDT PYYDVSRNA
SEQ ID No. 48: pre-pro-VIP-pDyn Hybrid:
MDTRNKAQLL VLLTLLSVLF SQTSAWPLYR APSALRLGDR IPFEGANEPD QVSLKEDIDM
LQNALAENDT PYYDVSRNAK RYGGFLRKYP KRSSEVAGEG DGDSMGHEDL
YKRYGGFLRR IRPKLKWDNQ KRYGGFLRRQ FKVVTRSQED PNAYSGELFD
SEQ ID No. 49: pre-pro CCK - N-peptide
MNSGVCLCVL MAVLAAGALT QPVPPADPAG SGLQRAEEAP RRQL
SEQ ID No. 50: pre-pro CCK-pDyn Hybrid
MNSGVCLCVL MAVLAAGALT QPVPPADPAG SGLQRAEEAP RRQLKRYGGF LRKYPKRSSE
VAGEGDGDSM GHEDLYKRYG GFLRRIRPKL KWDNQKRYGG FLRRQFKVVT
RSQEDPNAYS GELFD
SEQ ID No. 51: pre-pro Nociceptin - N-peptide
MKVLLCDLLL LSLFSSVFSS CQRDCLTCQE KLHPALDSFD LEVCILECEE KVFPSPLWTP
CTKVMARSSW QLSPAAPEHV AAALYQPRAS EMQHL
SEQ ID No. 52: pre-pro Nociceptin-pDyn Hybrid
MKVLLCDLLL LSLFSSVFSS CQRDCLTCQE KLHPALDSFD LEVCILECEE KVFPSPLWTP
CTKVMARSSW QLSPAAPEHV AAALYQPRAS EMQHLKRYGG FLRKYPKRSS
EVAGEGDGDS MGHEDLYKRY GGFLRRIRPK LKWDNQKRYG GFLRRQFKVV
TRSQEDPNAY SGELFD

Subject matter of the present invention is a delivery vector comprising a DNA sequence encoding pre-prodynorphin or pre-prodynorphin-variants according to the present invention wherein the modified propeptide fragment is optionally flanked by peptidase recognition signals comprising K, R, KR, RK or RR.

Peptidase (prohormone convertase) recognition signals are known to a person skilled in the art and may be single or paired basic amino acids, preferably but not exclusively K, R, KR, RK or RR.

According to the above description a specific pre-prodynorphin with shortened modified propeptide fragment may be the following:

SEQ: 53
MAWQGLVLAA CLLMFPSTTA DCLSRCSLCA VKTQDGPKPI
NPLICSLQCQ AALLPSEEWE RCQSFLSFFT PSTLGLNDKE
DLGSKSVGEG PYSELAKLSG SFLRKEQVKR YGGFLRKYPK
RSSEVAGEGD GDSMGHEDLY KRYGGFLRRI RPKLKWDNQK
RYGGFLRRQF KVVTRSQEDP NAYSGELFDA.

The bold amino acids may represent amino acids of the sorting motif of POMC and were derived from analogy, the italic amino acids represent Dyn A, the underlined amino acids represent leumorphin and the bold and underlined amino acids represent neoendorphin. Lastly, the amino acids in bold and italic represent peptidase recognition signals.

According to the above description a specific pre-prodynorphin with hybrid modified propeptide fragment may be the following:

Hybrid pPOMC - ppDyn (SEQ ID No. 54): [symbol]
KEQVKRYG
GFLRKYPKRS SEVAGEGDGD SMGHEDLYKR YGGFLRRIRP KLKWDNQKRY GGFLRRQFKV
VTRSQEDPNA YSGELFDA

The bold amino acids represent amino acids of the sorting motif of POMC, the italic amino acids represent Dyn A, the underlined amino acids represent leumorphin and the bold and underlined amino acids represent neoendorphin. Lastly, the amino acids in bold and italic represent peptidase recognition signals and the non-peptide coding part of ppDyn was replaced by parts of pPOMC (shaded in grey).

Subject matter of the present invention is a delivery vector comprising a DNA sequence encoding pre-prodynorphin or pre-prodynorphin-variants, wherein the target cells are neuronal cells of the central nervous system.

An embodiment of the present invention is a delivery vector comprising a DNA sequence encoding pre-prodynorphin or pre-prodynorphin-variants, wherein the target cells are subtypes of principal neurons and GABAergic interneurons.

Subject matter of the present invention is a delivery vector comprising a DNA sequence encoding pre-prodynorphin or pre-prodynorphin-variants wherein the signal peptide is a short peptide sequence of from 10 to 30 amino acids at the N-terminal end of precursor-proteins that are destined into the lumen of the endoplasmatic reticulum.

An embodiment of the present invention is a delivery vector comprising a DNA sequence encoding pre-prodynorphin or pre-prodynorphin-variants wherein the signal peptide is a short peptide sequence of from 10 to 30 amino acids, at the N-terminal end of precursor-proteins that are destined into the lumen of the endoplasmatic reticulum, wherein a stretch of 5 to 16 amino acids tends to form a single alpha helix structure.

The core of the signal peptide contains a stretch of hydrophobic amino acids (about 5 to 16 amino acids in length; that has a tendency to form a single alpha-helix and is also referred to as the “h-region”. In addition, many signal peptides begin with a positively charged stretch of amino acids, which may help to enforce proper topology of the polypeptide during translocation by what is known as the positive-inside rule. Yet, the amino-acid sequence of signal peptides strongly varies even within the group of pre-proneuropeptides.

Signal peptides as described herein can exhibit a number of varying sequences. Non-limiting examples of signal peptides in general, and signal peptides under the present invention may be selected, but are not restricted to the group comprising:

(SEQ ID No. 11)
MAWQGLVLAA CLLMFPSTTA
(SEQ ID No. 12)
MARFLTLCTW LLLLGPGLLA TVRA
(SEQ ID No. 13)
MLGNKRLGLS GLTLALSLLV CLGALAEA
(SEQ ID NO. 14)
MLSCRLQCAL AALSIVLALG CVTG
(SEQ ID No. 15)
MKILVALAVF FLVSTQLFA
(SEQ ID No. 16)
MRIMLLFTAI LAFSLA
(SEQ ID No. 17)
MPRSCCSRSG ALLLALLLQA SMEVRG
(SEQ ID No. 18)
MNSGVCLCVL MAVLAAGA
(SEQ ID No. 19)
MKVLLCDLLL LSLFSSVFS
(SEQ ID No. 20)
MQPTLLLSLL GAVGLAAVNS

For the avoidance of doubt according to the present invention sorting motif and signal peptide may be derived from the same pre-pro-neuropeptide or proteins sorted to large dense core vesicles, or from two different pre-pro-neuropeptides or proteins sorted to large dense core vesicles.

Subject matter of the present invention is a delivery vector, wherein said delivery vector leads to release-on-demand of dynorphins or dynorphin-variants with agonistic effects on human Kappa Opioid Receptors.

Subject matter of the present invention is a delivery vector wherein the variants have an amino acid sequence identity of at least 70% within the first 8 AA from the N-terminal end of SEQ ID No. 2 (YGGFLRRI), SEQ ID No. 3 (YGGFLRRQ) or SEQ ID No. 4 (YGGFLRRQ), respectively.

Subject matter of the present invention is a delivery vector, wherein the variants have an amino acid sequence identity of at least 80% within the first 8 AA from the N-terminal end of SEQ ID No. 2, SEQ ID No. 3 or SEQ ID No. 4, respectively.

Subject matter of the present invention is a delivery vector, wherein the variants have an amino acid sequence identity of at least 90% within the first 8 AA from the N-terminal end of SEQ ID No. 2, SEQ ID No. 3 or SEQ ID No. 4, respectively.

In a specific embodiment subject of the invention is a delivery vector as above described, wherein said delivery vector comprises multiple DNA sequences encoding SEQ ID No. 2, SEQ ID No. 3 and/or SEQ ID No. 4 or variants thereof wherein the sequences according to SEQ ID No. 2, SEQ ID No. 3 and/or SEQ ID No. 4 or variants thereof are flanked by peptidase recognition signals.

This means as an example that said delivery vector may comprise a DNA sequence encoding SEQ ID No. 2 two times in a way that two molecules of a peptide according to SEQ ID No. 2 would be derived from one delivery vector.

Peptidase (prohormone convertase) recognition signals are known to a person skilled in the art and may be single or paired basic amino acids, preferably but not exclusively K, R, KR, RK or RR.

In a specific embodiment subject of the invention is a delivery vector as above described, wherein said delivery vector comprises multiple DNA sequences encoding SEQ ID No. 2 and/or SEQ ID No. 4 or variants thereof wherein the sequences according, SEQ ID No. 2 and/or SEQ ID No. 4 or variants thereof are flanked by peptidase recognition signals.

Subject matter of the present invention is a delivery vector wherein said delivery vector comprises in addition a recombinant adeno-associated virus (AAV) vector genome or a recombinant lentivirus genome, particularly referring to DNA sequences based on such recombinant genomes.

The delivery vectors produced according to the present invention are useful for the delivery of nucleic acids to cells in vitro, ex vivo, and in vivo. In particular, the delivery vectors can be advantageously employed to deliver or transfer nucleic acids to animal, more preferably mammalian cells.

Suitable vectors include viral vectors (e.g., retrovirus, lentivirus, alphavirus; vaccinia virus; adenovirus, adeno-associated virus, or herpes simplex virus), lipid vectors, lipid nanoparticles, polylysine vectors, synthetic polyamino polymer vectors that are used with nucleic acid molecules, such as plasmids, and the like.

Any viral vector that is known in the art can be used in the present invention. Examples of such viral vectors include, but are not limited to vectors derived from: Adenoviridae; Adeno-associated Viridae (AAV), Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picomaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Tobovirus; Totiviridae; Group Tymovirus; and Plant virus satellites. Protocols for producing recombinant viral vectors and for using viral vectors for nucleic acid delivery can be found in (Ausubel et al., 1989) and other standard laboratory manuals (e.g., Rosenzweig et al. 2007). Particular examples of viral vectors are those previously employed for the delivery of nucleic acids including, for example, retrovirus, lentivirus, adenovirus, adeno-associated virus (AAV) and other parvoviruses, herpes virus, and poxvirus vectors. The term “parvovirus” as used herein encompasses the family Parvoviridae, including autonomous parvoviruses, densoviruses and dependoviruses. The term adeno-associated virus (AAV) includes all vertebrate variants especially of human, primate, other mammalian, avian or serpentine origin. The autonomous parvoviruses include members of the genera Parvovirus, Erythrovirus, Bocavirus, Densovirus, Iteravirus, and Contravirus. Exemplary autonomous parvoviruses include, but are not limited to, minute virus of mice, bovine parvovirus, canine parvovirus, chicken parvovirus, feline panleukopenia virus, feline parvovirus, goose parvovirus, HI parvovirus, muscovy duck parvovirus, bocavirus, bufavirus, tusavirus and B19 virus, and any other virus classified by the International Committee on Taxonomy of Viruses (ICTV) as a parvovirus. Other autonomous parvoviruses are known to those skilled in the art. See, e.g. (Berns et al. 2013).

In one embodiment of the invention said delivery vector comprises in addition a recombinant adeno-associated virus (AAV) vector genome or a recombinant lentivirus genome.

In one particular embodiment of the invention said delivery vector comprises in addition a recombinant AAV vector, wherein preferably said vector is a serotype of human or primate origin.

Subject matter of the present invention is a delivery vector comprising a recombinant adeno-associated virus (AAV) vector genome comprising inverted terminal repeats (ITR) preferably derived from AAV serotype 2, alternatively from AAV serotype 1, 3, 4, 5, 6, 7, 8, 9, 10, rh10, 11, 12, 13, 14, or reengineered variants thereof, wherein said vector genome is packaged in an AAV capsid selected from the group comprising AAV serotypes 1, 2, 3, 4, 5, 6, 7, 8, 9, 10, rh10, 11, 12, 13, 14, serpentine AAV, ancestral AAV, AAV-TT, AAVv66; AAV1P4, AAV1P5, AAV-PHP.B, AAV-PHP.eB, AAV2-HBKO, AAV.CAP-B10, AAV.CAP-MAC, AAV2.NN or further AAV capsid mutants derived thereof or a chimeric AAV vector comprising capsid proteins derived from two or more, preferably two of the aforementioned AAV serotype capsids; in particular embodiments, the capsids are capsids derived from AAV serotype 1 and/or 2, further particularly AAV1 and capsids derived thereof including AAV1P4, AAV1P5 or AAV2 and capsids derived thereof including AAV2-NN (Börner et al. 2020; Pavlou et al 2021; Challis et al. 2022; Naidoo et al 2018; Hsu et al. 2020; Tordo et al. 2018).

In particular embodiments of the present invention, the delivery vector comprising a recombinant adeno-associated virus (AAV) vector genome comprising inverted terminal repeats (ITR) preferably derived from AAV serotype 2, alternatively from AAV serotype 1, 3, 4, 5, 6, 7, 8, 9, 10, rh10, 11, 12, 13, 14, or reengineered variants thereof, wherein said vector genome is packaged in an AAV capsid selected from the group comprising AAV serotypes 1, 2, 3, 4, 5, 6, 7, 8, 9, 10, rh10, 11, 12, 13, 14, serpentine AAV, ancestral AAV, AAV-TT, AAVv66; AAV1P4, AAV1P5, AAV-PHP.B, AAV-PHP.eB, AAV2-HBKO, AAV.CAP-B10, AAV.CAP-MAC, AAV2.NN or further AAV capsid mutants derived thereof or chimeri, particularly of AAV serotype 1 or 2, further particularly AAV1 and capsids derived thereof including AAV1P4, AAV1P5 or AAV2 and capsids derived thereof including AAV2-NN (Bdrner et al. 2020; Pavlou et al 2021; Challis et al. 2022; Naidoo et al 2018; Hsu et al. 2020; Tordo et al. 2018).

Chimeric vectors (also known as mosaic vectors) and their methods of production are known from the scientific literature, e.g. Hauck et al., (2003), or as shown in Noé et al. (2008), and During et al. (2003). In certain embodiments, such chimeric vectors may improve vector yield in the production process or delivery of the vector and may allow for binding at multiple cell surface molecules serving as receptors (see e.g. herein below for further details). Furthermore, mosaic AAV capsids particularly mixtures of AAV2 and AAV1 were shown to lead to enhanced neurotropism upon CNS delivery in rodents and non-human primates with strongly reduced targeting of astrocytes or microglia, when compared to AAV1 only capsids (Kimura et al. 2023). Chimeric vectors comprise capsid proteins from more than one, typically two, different viral serotypes. The ratio between these different capsid proteins is can be chosen e.g. based on the desired cell targeting effect and/or the manufacturability or desired AAV vector yields; for instance, ratios for chimeric vectors comprising capsid proteins from two AAV capsids may be in the range of from 90:10 to 10:90; from 80:20 to 20:80, from 70:30 to 30:70; from 60:40 to 40:60; or about 50:50 (in each case meaning the protein ratio of the first AAV serotype capsid to the second AAV serotype capsid). In particular embodiments, such chimeric vectors comprise capsid proteins of AAV serotype 1 and capsid proteins of AAV serotype 2, such as the capsids derived therefrom as detailed above and in the ratios as detailed above, more particularly in a ratio of AAV2 capsid to AAV1 capsids of about 90:10.

In one particular embodiment of the invention said delivery vector is a single-stranded (ssAAV) vector or a self-complimentary vector (scAAV) also referred to as dimeric or duplex AAV vector (McCarty et al. 2001).

In one particular embodiment of the invention said delivery vector is a delivery vector as described above, wherein the DNA sequence encoding pre-prodynorphin or pre-prodynorphin-variants is operatively linked to expression control elements comprising a promoter and/or enhancer that induce sufficient expression of the gene product of interest to obtain a therapeutic effect.

For example, the encoding nucleic acid may be operably associated with expression control elements, such as promoters, enhancers, other transcription/translation control signals, origins of replication, polyadenylation signals, and/or internal ribosome entry sites (IRES) and the like. It will further be appreciated that a variety of promoter/enhancer elements may be used depending on the level and tissue-specific expression desired. The promoter/enhancer may be constitutive or inducible, depending on the pattern of expression desired. The promoter/enhancer may be native or foreign and can be a natural or a synthetic sequence. By foreign, it is intended that the transcriptional initiation region is not found in the wild-type host into which the transcriptional initiation region is introduced. Promoter/enhancer elements that are functional in the target cell or subject to be treated are most preferred. Mammalian promoter/enhancer elements are also preferred. Most preferred are promoter/enhancer elements active in human neurons and not, or to a lesser extend in glial cells. The promoter/enhancer element may express the transgene constitutively or inducibly.

Exemplary constitutive promoters include, but are not limited to a Beta-actin promoter, a cytomegalovirus promoter, a cytomegalovirus-enhancer/chicken beta-actin hybrid promoter, and a Rous sarcoma virus promoter. Inducible expression control elements are generally employed in those applications in which it is desirable to provide regulation over expression of the heterologous nucleic acid sequence(s). Inducible promoters/enhancer elements for gene delivery include neuron-specific, brain-specific, muscle specific (including cardiac, skeletal and/or smooth muscle), liver specific, bone marrow specific, pancreatic specific, spleen specific, and lung specific promoter/enhancer elements. In particular embodiments, the promoter/enhancer is functional in cells or tissue of the CNS and may even be specific to cells or tissues of the CNS. Such promoters/enhancers include but are not limited to promoters/enhancers that function in the eye (e.g., retina and cornea), neurons (e.g., the neuron specific enolase, AADC, human synapsin (hSYN), phosphoglycerate kinase (PGK), or serotonin receptor promoter), glial cells (e.g., S100 or glutamine synthase promoter), and oligodendrocytes. Other promoters that have been demonstrated to induce transcription in the CNS include, but are not limited to, myelin basic protein (MBP) promoter (Tani et al., 1996), and the prion promoter (Loftus et al., 2002). Preferred is a neuron-specific promoter displaying significantly reduced, preferably no expression in glial cells.

Other inducible promoter/enhancer elements include drug-inducible, hormone-inducible and metal-inducible elements, and other promoters regulated by exogenously supplied compounds, including without limitation, the zinc-inducible metallothionein (MT) promoter; the dexamethasone (Dex)-inducible mouse mammary tumor virus (MMTV) promoter; the T7 polymerase promoter system (see WO 98/10088); the ecdysone-inducible insect promoter (No et al, 1996); the tetracycline-repressible system (Gossen and Bujard, 1992); the tetracycline-inducible system (Gossen et al., 1995); see also (Harvey et al., 1998); the RU486-inducible system (Wang, DeMayo et al., 1997); (Wang, Xu et al., 1997); and the rapamycin-inducible system (Magari et al., 1997).

In a particular embodiment of the invention the promoter and/or enhancer is selected from the group comprising constitutively active promoters e.g. CMV (cytomegalovirus immediate-early gene enhancer/promoter)- or CBA promoter (chicken beta actin promoter and human cytomegalovirus IE gene enhancer), or inducible promoters comprising Gene Switch, tet-operon derived promotor, or neuron-specific promoters derived of e.g. phosphoglycerate kinase (PGK), synapsin-1 (SYN), neuron-specific enolase (NSE), preferably but not exclusively of human origin.

In a particular embodiment of the invention said delivery vector further comprises a posttranscriptional regulatory element, preferably the woodchuck-hepatitis-virus-posttranscriptional-regulatory element (WPRE) or shortened variants derived thereof (Loeb et al. 1999; Choi et al. 2014). Other possible posttranscriptional regulatory elements are known to a person skilled in the art.

Subject matter of the present invention is a recombinant virus particle or a liposome or a nanoparticle comprising a delivery vector according to the invention.

Subject matter of the present invention is the recombinant virus particle or liposome, or nanoparticle wherein said delivery vector comprises in addition a recombinant adeno-associated virus (AAV) vector genome and said rAAV vector genome is encapsidated in an AAV capsid or wherein said delivery vector comprises in addition a recombinant lentivirus vector genome and is packaged in a lentivirus particle.

For sake of completeness, it is apparent that as used herein the terms encapsidated and packaged with respect to virus particles may in instances be used interchangeably and refer to polynucleotides (i.e. vectors, genomic DNA, etc.) being contained in said capsid or virus particle.

Subject of the present invention is furthermore a recombinant gene therapy vector comprising the foreign, therapeutic coding sequence, which is flanked by genetic elements for its expression and by virus-specific cis elements for its replication, genome packaging, genomic integration etc. The said virus genome is encapsidated as virus particle consisting of virus-specific proteins as in the case of AAV. In the case of lentivirus vectors the viral genome and virus-specific proteins, like reverse transcriptase and others are encapsidated into lentivirus capsids. These are enveloped by a lipid bilayer into which virus-specific proteins are embedded. Liposomes comprise the above-described nucleotide sequences or entire DNA backbones including all regulatory elements of the gene therapy-, or delivery vector.

Examples of liposomes include DSPC (1,2-distearoyl-sn-glycero-3-phosphocholine, cholesterol, DSPE-PEG2000 (1,2-distearoyl-sn-glycero-3-phosphoethanol-amine-N-[amino(polyethylene glycol)-2000], or DSPE-PEG2000-mal (1,2-distearoyl-sn-glycero-3-phosphoethanol-amine-N-[maleimide(polyethylene glycol)-2000] or variants comprising sphingomyelin/cholesterol and phosphatidic acid.

In one particular embodiment of the invention said delivery vector comprises in addition a recombinant adeno-associated virus (AAV) vector genome and said recombinant AAV (rAAV) vector genome is encapsidated in an AAV capsid.

Adeno-associated viruses (AAV) have been developed as nucleic acid delivery vectors. For a review, see (Muzyczka, 1992) (Li and Samulski, 2020). AAV are helper-dependent parvoviruses requiring a helper virus, typically adenovirus or herpesvirus for productive replication. AAV represent a growing family of at least 14 naturally occurring serotypes of human or primate origin. AAVs of other mammalian species, or of avian or insect origin have been described (see Berns et al., 2013). The AAVs have small icosahedral capsids, 18-26 nanometers in diameter and contain a single-stranded DNA genome of 4-5 kilobases in length. AAV encapsidates both AAV DNA strands, either the sense or antisense DNA strand is incorporated into one virion. The AAV genome carries two major open reading frames encoding the genes rep and cap. Rep encodes a family of overlapping, nonstructural, regulatory proteins. In the best-studied AAV prototype strain, AAV2, the mRNAs for Rep78 and Rep68 are transcribed from the AAV p5 promoter (Stutika et al. 2015). Rep78/68 are required for AAV transcription, AAV DNA replication, AAV integration into the host cell genome and its rescue therefrom. Rep52 and Rep40 represent N-terminally truncated versions of Rep78 and Rep68 transcribed from a separate promoter, p19 and are required for encapsidation of the newly synthesized AAV genome into preformed AAV capsids. These are formed by the three cap gene-derived proteins, VP1, VP2, and VP3. The cap ORF also encodes AAP, an assembly-enhancing protein, and an AAV egress-promoting factor called MAAP. AAP and MAAP do not form part of the capsid (Sonntag et al. 2010; Elmore et al. 2021). The AAV ORFs are flanked by inverted terminal repeat sequences (ITRs) at either end of the genome. These vary in length between AAV serotypes, in AAV2 these comprise around 145 bp, the first 125 bp thereof are capable of forming Y- or T-shaped duplex structures. The ITRs comprise terminal resolution sites (trs) where the replicated concatemeric AAV genome is nicked by Rep to form unit length ssAAV genomes ready for packaging into AAV capsids. The ITRs represent the minimal AAV sequences required in cis for DNA replication, packaging, genomic integration and rescue. Only these have to be retained in an AAV vector to ensure DNA replication and packaging of the AAV vector genome. Foreign genes flanked by AAV-ITRs will be replicated and packaged into AAV capsids provided the AAV genes rep and cap are expressed in trans in the chosen packaging cell (Muzyczka, 1992). In the case of scAAV the terminal resolution site (trs) is deleted in one of the ITRs, so that the AAV genome cannot be nicked by Rep on the affected end, thereby being retained as unresolved duplex, self-complementary (sc)AAV genome.

AAV are among the few viruses that can persist over months and years in non-dividing cells in vivo, including neurons, muscle, liver, heart and others. Wildtype AAV2 has been shown to integrate its genome into the host cell genome in a Rep78/68-dependent manner, with a preference for chromosomal loci with DNA sequence homology to the so-called Rep-binding site which forms part of the AAV-ITRs (Hiser et al. 2014). In contrast, AAV vectors mostly persist as concatemeric nuclear episomes. Devoid of the AAV genes rep and cap AAV vectors rarely integrate at all, and if so without genomic preference (Hiser et al. 2014). Nonetheless long term AAV persistence has been shown in non-dividing, postmitotic cells including neurons which renders AAV vectors ideal for CNS transduction and long-term gene addition therapy of chronic diseases of genetic or acquired origin.

Generally, a recombinant AAV vector (rAAV) genome will only retain the inverted terminal repeat (ITR) sequence(s) in its native (ssAAV) or trs-deleted (scAAV) version, so as to maximize the size of the transgene that can be efficiently packaged by the vector. The structural- and non-structural protein-coding sequences may be provided in trans, e.g., from a vector, such as a plasmid, by stably integrating the respective genes into a packaging cell, or in a recombinant helper virus such as HSV or baculovirus, as reviewed in (Mietzsch, Grasse et al., 2014). Typically, the rAAV vector genome comprises at least one AAV inverted terminal repeat (ITR), more typically two AAV inverted terminal repeats, which will generally be at the 5′ and 3′ ends of the heterologous nucleotide sequence(s). The AAV ITR may be from any AAV including serotypes 1-14. Since AAV2-derived ITRs can be cross-packaged into virtually any AAV serotype capsids, AAV2 ITRs combined with AAV2 rep are mostly employed. The AAV terminal repeats need not maintain the wild-type terminal repeat sequence (e.g., a wild-type sequence may be altered by insertion, deletion, truncation or missense mutations), as long as the terminal repeat mediates the desired functions, e.g., DNA replication, virus packaging, integration, and/or provirus rescue, and the like. The rAAV vector genome spans generally about 70% to about 105% of the size of the wild-type genome and comprises an appropriate packaging signal as part of the AAV-ITR. To facilitate packaging into an AAV capsid, the entire vector genome (from ITR to ITR) is preferably below 5.2 kb, more preferably up to 4.8 kb in size to allow packaging of the entire recombinant genome into the preformed AAV capsid. So-called dimeric or self-complementary AAV vectors (scAAV) were developed to package double-stranded instead of single-stranded AAV genomes (McCarty et al., 2001). scAAVs lead to enhanced AAV gene expression, however at the price of reduced transgene capacity. The total packaging capacity is only 2.4 kb (from ITR to ITR), which is enough for small genes or cDNAs including those for neuropeptides.

Any suitable method known in the art can be used to produce AAV vectors expressing the nucleic acids of this invention. AAV vector stocks can be produced by co-transfection of plasmids for the ITR-flanked AAV vector genome expressing the transgene together with an AAV rep/cap expressing plasmid of the desired serotype and adenovirus-derived helper genes for AAV replication (Grimm et al., 2003; Xiao et al., 1998). AAV vectors can also be produced in packaging cell lines of mammalian or insect origin and/or in combination with recombinant helper viruses, such as adenovirus, herpes simplex virus (HSV), another member of the herpesvirus family, or baculovirus, as reviewed and discussed in (Mietzsch, Grasse et al., 2014).

Subject matter of the present invention is a delivery vector comprising a DNA sequence encoding pre-prodynorphin or pre-prodynorphin-variants as detailed herein.

Subject matter of the present invention is a delivery vector or recombinant virus particle or liposome or nanoparticle for use in delivering a nucleic acid to a cell of the central nervous system, comprising contacting the cell with the delivery vector or recombinant virus particle or liposome or nanoparticle under conditions sufficient for the DNA sequence encoding pre-prodynorphin or pre-prodynorphin-variants to be introduced into the cell, more specifically into the cell nucleus.

The delivery vectors of the present invention provide a means for delivering nucleic acid sequences into cells of the central nervous system, preferably neurons. The delivery vectors may be employed to transfer a nucleotide sequence of interest to a cell in vitro, e.g., to produce a polypeptide in vitro or for ex vivo gene therapy. The vectors are additionally useful in a method of delivering a nucleotide sequence to a subject in need thereof. In this manner, the polypeptide may thus be produced in vivo in the subject. The subject may be in need of the polypeptide because the subject has a deficiency of the polypeptide, or because the production of the polypeptide in the subject may impart some therapeutic effect, as a method of treatment or otherwise, and as explained further below.

In one particular embodiment of the method of delivering a nucleic acid to a cell of the central nervous system the pre-prodynorphin or pre-prodynorphin-variant is produced processed and mature dynorphin peptides or variants thereof released from the cell.

In one particular embodiment of the method of delivering a nucleic acid to a cell of the central nervous system the method comprises contacting the cell with the recombinant virus particle or liposome or nanoparticle as described above under conditions sufficient for the DNA sequence encoding pre-prodynorphin or pre-prodynorphin-variants to be introduced into the cell nucleus. Conditions sufficient for the DNA sequence encoding pre-prodynorphin or pre-prodynorphin-variants to be introduced into the cell is the contacting of the AAV capsid to host cell surface receptors and coreceptors. AAV1 capsids bind to 2-3 sialic acid linked to N-acetylgalactosamine, followed by 1-4-linked N-acetylglucosamine, whereas AAV2 capsids bind to heparin sulfate proteoglycan particularly 6-O- and N-sulfated heparins on the cell surface (Mietzsch, Broecker et al., 2014). AAV coreceptors include FGFR-1, Integrin aVb5, hepatocyte growth factor receptor (c-met) and the universal AAV receptor, AAVR necessary for transduction with AAV1, AAV2 and other serotypes irrespective of the presence of specific glycans (Pillay et al., 2016). AAVR directly binds to AAV particles and helps trafficking to the trans Golgi network. AAV2 has been described to use the nuclear pore complex for nuclear entry thereby interacting with importin-p alone or in complex with other import proteins (Nicolson and Samulski 2014). Most parvoviruses and AAV serotypes use similar mechanisms for nuclear entry (Mattola et al. 2022), AAV vectors are assembled in the cell nucleus.

Subject matter of the present invention is a delivery vector or recombinant virus particle or liposome or nanoparticle for use as medicament.

Subject matter of the present invention is a delivery vector or recombinant virus particle or liposome or nanoparticle as detailed herein for use in treating focal epilepsy in a subject, in particular mesial temporal lobe epilepsy, or for use in preventing epileptic seizures in a subject that suffers from focal epilepsy through activation of human Kappa Opioid Receptors in the epileptogenic focus, thereby inhibiting seizures.

Particularly, the delivery vector or recombinant virus particle or liposome or nanoparticle as detailed herein is able to deliver a DNA sequence encoding pre-prodynorphin or pre-prodynorphin-variants as described herein, which in turn drives expression of a pre-propeptide in a target cell, enabling the release of dynorphin or dynorphin-variants from the target cell on demand as described herein, thereby leading to activation of human Kappa Opioid Receptors in the epileptogenic focus.

Subject matter of the present invention is a delivery vector or recombinant virus particle or liposome or nanoparticle for use in treating focal epilepsy in a subject, in particular mesial temporal lobe epilepsy, or for use in preventing epileptic seizures in a subject that suffers from focal epilepsy through activation of human Kappa Opioid Receptors in the epileptogenic focus, thereby inhibiting seizures and/or through to on-demand release of peptides with agonistic effects on human Kappa Opioid Receptors in the epileptogenic focus.

Subject matter of the present invention is a delivery vector or recombinant virus particle or a liposome or nanoparticle for use in treating focal epilepsy in a subject, in particular mesial temporal lobe epilepsy, or for use in preventing epileptic seizures in a subject that suffers from focal epilepsy wherein said vector or recombinant virus particle or liposome or nanoparticle is suitable for peripheral administration or for intracranial or for intracerebral or for intrathecal or for intraparenchymal administration.

Subject matter of the present invention is a delivery vector or recombinant virus particle or a liposome or nanoparticle for use in treating focal epilepsy in a subject, in particular mesial temporal lobe epilepsy, or for use in preventing epileptic seizures in a subject that suffers from focal epilepsy, wherein said delivery vector or recombinant virus particle or a liposome or nanoparticle is applied intracerebral, preferred is applied focal.

Subject matter of the present invention is a pharmaceutical release-on-demand composition, delivery vector or recombinant virus particle or liposome or nanoparticle, and optionally a pharmaceutically acceptable carrier.

Subject matter of the present invention is a cell infected, preferably in vitro or ex vivo, with a delivery vector or recombinant virus or liposome or nanoparticle.

Subject matter of the present invention is a method of treating a subject with focal epilepsy in particular mesial temporal lobe epilepsy, or a method of preventing epileptic seizures in a subject that suffers from focal epilepsy comprising administering a delivery vector, a recombinant virus particle or a liposome or nanoparticle, or a pharmaceutical composition to the subject, whereby preferably said delivery vector or recombinant virus particle or liposome or nanoparticle encode pre-propeptides, which after maturation and release provide activation of human Kappa Opioid Receptors in the epileptogenic focus, thereby inhibiting seizures, and wherein preferably said delivery vector or recombinant virus particle or a liposome or nanoparticle is applied intracerebral, intraparenchymal, preferably applied focal.

### Description of the Below Sequences:

SEQ ID No. 1
(ppDyn)
MAWQGLVLAA CLLMFPSTTA DCLSRCSLCA VKTQDGPKPI
NPLICSLQCQ AALLPSEEWE RCQSFLSFFT PSTLGLNDKE
DLGSKSVGEG PYSELAKLSG SFLKELEKSK FLPSISTKEN
TLSKSLEEKL RGLSDGFREG AESELMRDAQ LNDGAMETGT
LYLAEEDPKE QVKRYGGFLR KYPKRSSEVA GEGDGDSMGH
EDLYKRYGGF LRRIRPKLKW DNQKRYGG FLRRQFKVVT
RSQEDPNAYS GELFDA

Human pre-prodynorphin before processing as expressed in the human brain.

SEQ ID No. 2 Dyn A
YGGFLRRIRPKLKWDNQ
SEQ ID No. 3 Dyn B (rimorphin)
YGGFLRRQFKVVT
SEQ ID No. 4: Leumorphin
YGGFLRRQFKVVTRSQEDPNAYSGELFDA
SEQ ID No. 5: Unmodified propeptide fragment of ppDyn
DCLSRCSLCA VKTQDGPKPI NPLICSLQCQ AALLPSEEWE RCQSFLSFFT PSTLGLNDKE
DLGSKSVGEG PYSELAKLSG SFLKELEKSK FLPSISTKEN TLSKSLEEKL RGLSDGFREG
AESELMRDAQ LNDGAMETGT LYLAEEDPKE QV
SEQ ID No. 6: Modified propeptide fragment of ppDyn
DCLSRCSLCA VKTQDGPKPI NPLICSLQCQ AALLPSEEWE RCQSFLSFFT PSTLGLNDKE
DLGSKSVGEG PYSELAKLSGSFLRKEQVKR
SEQ ID No. 7 Modified propeptide fragment of ppDyn
DLGSKSVGEG PYSELAKLSG SFLKELEKSK FLPSISTKEN TLSKSLEEKL RGLSDGFREG
AESELMRDAQ LNDGAMETGT LYLAEEDPKE QV
SEQ ID No. 8 Modified propeptide fragment of ppDyn
DLGSKSVGEG PYSELAKLSG SFLRKE QV
SEQ ID No. 9 Modified propeptide fragment of ppDyn
DLGSKSVGEG PYSELAKLRKE QV
SEQ ID No. 10: N-terminal part of pPOMC
MPRSCCSRSG ALLLALLLQA SMEVRGWCLE SSQCQDLTTE SNLLECIRAC KP
SEQ ID No. 11: Signal peptide of ppdynorphin
MAWQGLVLAA CLLMFPSTTA
SEQ ID No. 12: Signal peptide of ppenkephalin
MARFLTLCTW LLLLGPGLLA TVRA
SEQ ID No. 13: Signal peptide of ppneuropeptide Y
MLGNKRLGLS GLTLALSLLV CLGALAEA
SEQ ID No. 14: Signal peptide of ppsomatostatin
MLSCRLQCAL AALSIVLALG CVTG
SEQ ID No. 15: Signal peptide of pptachykinin A
MKILVALAVF FLVSTQLFA
SEQ ID No. 16: Signal peptide of pptachykinin B
MRIMLLFTAI LAFSLA
SEQ ID No. 17: Signal peptide of pproopiomelanocortin
MPRSCCSRSG ALLLALLLQA SMEVRG
SEQ ID No. 18: Signal peptide of ppcholecystokinin
MNSGVCLCVL MAVLAAGA
SEQ ID No. 19: Signal peptide of ppnociceptin
MKVLLCDLLL LSLFSSVFS
SEQ ID No. 20: Signal peptide of ppchromogranin B
MQPTLLLSLL GAVGLAAVNS
Sorting Motif: SEQ ID No. 36
DLXₓEX_{y}L

wherein x is an integer from 1 to 20, y is an integer from 1 to 10, and each instance of X may independently be any amino acid.

```
SEQ ID No. 37:
pre-proenkephalin-N-peptide
MARFLTLCTW LLLLGPGLLA TVRAECSQDC ATCSYRLVRP ADINFLACVM ECEGKLPSLK
IWETCKELLQ LSKPELPQDG TSTLRENSKP EESHLLA
SEQ ID No. 38:
pre-proenkephalin-pDyn Hybrid
MARFLTLCTW LLLLGPGLLA TVRAECSQDC ATCSYRLVRP ADINFLACVM ECEGKLPSLK
IWETCKELLQ LSKPELPQDG TSTLRENSKP EESHLLAKRY GGFLRKYPKR SSEVAGEGDG
DSMGHEDLYK RYGGFLRRIR PKLKWDNQKR YGGFLRRQFK VVTRSQEDPN AYSGELFDA
SEQ ID No. 39:
pre-pro-NPY-N-peptide
MLGNKRLGLS GLTLALSLLV CLGALAEA
SEQ ID No. 40:
pre-pro-NPY-pDyn Hybrid
MLGNKRLGLS GLTLALSLLV CLGALAEAKR YGGFLRKYPK RSSEVAGEGD
GDSMGHEDLY KRYGGFLRRI RPKLKWDNQK RYGGFLRRQF KVVTRSQEDP NAYSGELFD
SEQ ID No. 41:
pre-pro BDNF-N-peptide
MTILFLTMVI SYFGCMKAAP MKEANIRGQG GLAYPGVRTH GTLESVNGPK AGSRGLTSLA
DTFEHVIEEL LDEDHKVRPN EENNKDADLY TSRVMLSSQV PLEPPLLFLL EEYKNYLDAA
NMSM
SEQ ID No. 42:
pre-pro BDNF-pDyn Hybrid
MTILFLTMVI SYFGCMKAAP MKEANIRGQG GLAYPGVRTH GTLESVNGPK AGSRGLTSLA
DTFEHVIEEL LDEDHKVRPN EENNKDADLY TSRVMLSSQV PLEPPLLFLL EEYKNYLDAA
NMSMKRYGGF LRKYPKRSSE VAGEGDGDSM GHEDLYKRYG GFLRRIRPKL
KWDNQKRYGG FLRRQFKVVT RSQEDPNAYS GELFD
SEQ ID No. 43:
pre-pro-Somatostatin-N-peptide
MLSCRLQCAL AALSIVLALG CVTGAPSDPR LRQFLQKSLA AAAGKQELAK YFLAELLSEP
NQTENDALEP EDLSQAAEQD EMRLELQR
SEQ ID No. 44:
pre-pro-Somatostatin-pDyn Hybrid
MLSCRLQCAL AALSIVLALG CVTGAPSDPR LRQFLQKSLA AAAGKQELAK YFLAELLSEP
NQTENDALEP EDLSQAAEQD EMRLELQRKR YGGFLRKYPK RSSEVAGEGD
GDSMGHEDLY KRYGGFLRRI RPKLKWDNQK RYGGFLRRQF KVVTRSQEDP NAYSGELFD
SEQ ID No. 45:
pre-pro-Tachykinin A-N-peptide
MKILVALAVF FLVSTQLFAE EIGANDDLNY WSDWYDSDQI KEELPEPFEH LLQRIA
SEQ ID No. 46:
pre-pro-Tachykinin A-pDyn Hybrid
MKILVALAVF FLVSTQLFAE EIGANDDLNY WSDWYDSDQI KEELPEPFEH LLQRIAKRYG
GFLRKYPKRS SEVAGEGDGD SMGHEDLYKR YGGFLRRIRP KLKWDNQKRY
GGFLRRQFKV VTRSQEDPNA YSGELFD
SEQ ID No. 47:
pre-pro-VIP-N-peptide
MDTRNKAQLL VLLTLLSVLF SQTSAWPLYR APSALRLGDR IPFEGANEPD QVSLKEDIDM
LQNALAENDT PYYDVSRNA
SEQ ID No. 48:
pre-pro-VIP-pDyn Hybrid
MDTRNKAQLL VLLTLLSVLF SQTSAWPLYR APSALRLGDR IPFEGANEPD QVSLKEDIDM
LQNALAENDT PYYDVSRNAK RYGGFLRKYP KRSSEVAGEG DGDSMGHEDL
YKRYGGFLRR IRPKLKWDNQ KRYGGFLRRQ FKVVTRSQED PNAYSGELFD
SEQ ID No. 49:
pre-pro CCK-N-peptide
MNSGVCLCVL MAVLAAGALT QPVPPADPAG SGLQRAEEAP RRQL
SEQ ID No. 50:
pre-pro CCK-pDyn Hybrid
MNSGVCLCVL MAVLAAGALT QPVPPADPAG SGLQRAEEAP RRQLKRYGGF
LRKYPKRSSEV AGEGDGDSMG HEDLYKRYGG FLRRIRPKLK WDNQKRYGGF
LRRQFKVVTR SQEDPNAYSG ELFD
SEQ ID No. 51:
pre-pro Nociceptin-N-peptide
MKVLLCDLLL LSLFSSVFSS CQRDCLTCQE KLHPALDSFD LEVCILECEE KVFPSPLWTP
CTKVMARSSW QLSPAAPEHV AAALYQPRAS EMQHL
SEQ ID No. 52:
pre-pro Nociceptin-pDyn Hybrid
MKVLLCDLLL LSLFSSVFSS CQRDCLTCQE KLHPALDSFD LEVCILECEE KVFPSPLWTP
CTKVMARSSW QLSPAAPEHV AAALYQPRAS EMQHLKRYGG FLRKYPKRSS
EVAGEGDGDS MGHEDLYKRY GGFLRRIRPK LKWDNQKRYG GFLRRQFKVV
TRSQEDPNAY SGELFD
Shortened modified ppDyn SEQ ID No. 53
MAWQGLVLAA CLLMFPSTTA DCLSRCSLCA VKTQDGPKPI NPLICSLQCQ AALLPSEEWE
RCQSFLSFFT PSTLGLNDKE DLGSKSVGEG PYSELAKLSGS FLRKEQVKRY GGFLRKYPKR
SSEVAGEGDG DSMGHEDLYK RYGGFLRRIR PKLKWDNQKR YGGFLRRQFK VVTRSQEDPN
AYSGELFDA.
Hybrid pPOMC-ppDyn: SEQ ID No 54
MPRSCCSRSG ALLLALLLQA SMEVRGWCLE SSQCQDLTTE SNLLECIRAC KPKEQVKRYG
GFLRKYPKRS SEVAGEGDGD SMGHEDLYKR YGGFLRRIRP KLKWDNQKRY GGFLRRQFKV
VTRSQEDPNA YSGELFDA
Modified propeptide fragments of ppDyn
SEQ ID No. 55
DLGSKSVGEG PYSELRKEQV.
SEQ ID No.: 56
DLGSKSVGEG PYSELAKLSG SFLKKEQV
SEQ ID No.: 57
DLGSKSVGEG PYSELAKL
SEQ ID No.: 58
DLGSKSVGEG PYSEL
SEQ ID No. 59:
SSAAV left ITR (145 bp)
      1 TTGGCCACTC CCTCTCTGCG CGCTCGCTCG CTCACTGAGG CCGGGCGACC AAAGGTCGCC
    61 CGACGCCCGG GCTTTGCCCG GGCGGCCTCA GTGAGCGAGC GAGCGCGCAG AGAGGGAGTG
  121 GCCAACTCCA TCACTAGGGG TTCCT
SEQ ID No. 60:
SSAAV/scAAV right ITR (145 bp)
      1 AGGAACCCCT AGTGATGGAG TTGGCCACTC CCTCTCTGCG CGCTCGCTCG CTCACTGAGG
    61 CCGGGCGACC MAAGGTCGCC CGACGCCCGG GCTTTGCCCG GGCGGCCTCA GTGAGCGAGC
  121 GAGCGCGCAG AGAGGGAGTG GCCAA
SEQ ID No. 61:
scAAV left ITR(Δtrs) (121 bp)
      1 TTGGCCACTC CCTCTCTGCG CGCTCGCTCG CTCACTGAGG CCGGGCGACC AAAGGTCGCC
    61 CGACGCCCGG GCTTTGCCCG GGCGGCCTCA GTGAGCGAGC GAGCGCGCAG AGAGGGAGTG
  121 G
SEQ ID No. 63:
CBA Promoter: CMV-enhancer, chicken beta-actin promoter, chimeric
intron (887 bp)
      1 TAATAGTAAT CAATTACGGG GTCATTAGTT CATAGCCCAT ATATGGAGTT CCGCGTTACA
    61 TAACTTACGG TAAATGGCCC GCCTGGCTGA CCGCCCAACG ACCCCCGCCC ATTGACGTCA
  121 ATAATGACGT ATGTTCCCAT AGTAACGCCA ATAGGGACTT TCCATTGACG TCAATGGGTG
  181 GAGTATTTAC GGTAAACTGC CCACTTGGCA GTACATCAAG TGTATCATAT GCCAAGTACG
  241 CCCCCTATTG ACGTCAATGA CGGTAAATGG CCCGCCTGGC ATTATGCCCA GTACATGACC
  301 TTATGGGACT TTCCTACTTG GCAGTACATC TACGTATTAG TCATCGCTAT TACCATGGTC
  361 GAGGTGAGCC CCACGTTCTG CTTCACTCTC CCCATCTCCC CCCCCTCCCC ACCCCCAATT
  421 TTGTATTTAT TTATTTTTTA ATTATTTTGT GCAGCGATGG GGGCGGGGGG GGGGGGGGGG
  481 CGCGCGCCAG GCGGGGGGGG GCGGGGCGAG GGGCGGGGCG GGGCGAGGCG GAGAGGTGCG
  541 GCGGCAGCCA ATCAGAGCGG CGCGCTCCGA AAGTTTCCTT TTATGGCGAG GCGGCGGCGG
  601 CGGCGGCCCT ATAAAAAGCG AAGCGCGCGG CGGGCGGGAG TCGCTGCGCG CTGCCTTCGC
  661 CCCGTGCCCC GCTCCGCCGC CGCCTCGCGC CGCCCGCCCC GGCTCTGACT GACCGCGTTA
  721 CTCCCACAGG TGAGCGGGCG GGACGGCCCT TCTCCTCCGG GCTGTAATTA GCGCTTGGTT
  781 TAATGACGGC TTGTTTCTTT TCTGTGGCTG CGTGAAAGCC TTGAGGGGCT CCGGGAGGGC
  841 CCTAGAGCCT CTGCTAACCA TGTTCATGCC TTCTTCTTTT TCCTACA
SEQ ID No. 64:
sCBA-Promoter:
CMV-enhancer, chicken-beta actin promoter, chimeric
intron (851 bp)
      1 CCATATATGG AGTTCCGCGT TACATAACTT ACGGTAAATG GCCCGCCTGG CTGACCGCCC
    61 AACGACCCCC GCCCATTGAC GTCAATAATG ACGTATGTTC CCATAGTAAC GCCAATAGGG
  121 ACTTTCCATT GACGTCAATG GGTGGAGTAT TTACGGTAAA CTGCCCACTT GGCAGTACAT
  281 CAAGTGTATC ATATGCCAAG TACGCCCCCT ATTGACGTCA ATGACGGTAA ATGGCCCGCC
  241 TGGCATTATG CCCAGTACAT GACCTTATGG GACTTTCCTA CTTGGCAGTA CATCTACGTA
  301 TTAGTCATCG CTATTACCAT GGTCGAGGTG AGCCCCACGT TCTGCTTCAC TCTCCCCATC
  361 TCCCCCCCCT CCCCACCCCC AATTTTGTAT TTATTTATTT TTTAATTATT TTGTGCAGCG
  421 ATGGGGGGGG GGGGGGGGGG GGGGCGCGCG CCAGGCGGGG CGGGGGGGGG CGAGGGCGGG
  481 GGCGGGGCGA GGCGGAGAGG TGCGGCGGCA GCCAATCAGA GCGGCGCGCT CCGAAAGTTT
  541 CCTTTTATGG CGAGGCGGCG GCGGCGGCGG CCCTATAAAA AGCGAAGCGC GCGGCGGGCG
  601 GGAGTCGCTG CGCGCTGCCT TCGCCCCGTG CCCCGCTCCG CCGCCGCCTC GCGCCGCCCG
  661 CCCCGGCTCT GACTGACCGC GTTACTCCCA CAGGTGAGCG GGGGGGACGG CCCTTCTCCT
  721 CCGGGCTGTA ATTAGCGCTT GGTTTAATGA CGGCTTGTTT CTTTTCTGTG GCTGCGTGAA
  781 AGCCTTGAGG GGCTCCGGGA GGGCCCTAGA GCCTCTGCTA ACCATGTTCA TGCCTTCTTC
  841 TTTTTCCTAC A
SEQ ID No. 65:
Human synapsin-promoter (448 bp)
      1 AGTGCAAGTG GGTTTTAGGA CCAGGATGAG GCGGGGTGGG GGTGCCTACC TGACGACCGA
    61 CCCCGACCCA CTGGACAAGC ACCCAACCCC CATTCCCCAA ATTGCGCATC CCCTATCAGA
  121 GAGGGGGAGG GGAAACAGGA TGCGGCGAGG CGCGTGCGCA CTGCCAGCTT CAGCACCGCG
  181 GACAGTGCCT TCGCCCCCGC CTGGCGGCGC GCGCCACCGC CGCCTCAGCA CTGAAGGCGC
  241 GCTGACGTCA CTCGCCGGTC CCCCGCAAAC TCCCCTTCCC GGCCACCTTG GTCGCGTCCG
  301 CGCCGCCGCC GGCCCAGCCG GACCGCACCA CGCGAGGCGC GAGATAGGGG GGCACGGGCG
  361 CGACCATCTG CGCTGCGGCG CCGGCGACTC AGCGCTGCCT CAGTCTGCGG TGGGCAGCGG
  421 AGGAGTCGTG TCGTGCCTGA GAGCGCAG
SEQ ID No. 66:
WPRE:
Woodchuck hepatitis virus posttranscriptional regulatory
element (582 bp)
      1 CTCTGGATTA CAAAATTTGT GAAAGATTGA CTGGTATTCT TAACTATGTT GCTCCTTTTA
    61 CGCTATGTGG ATACGCTGCT TTAATGCCTT TGTATCATGC TATTGCTTCC CGTATGGCTT
  121 TCATTTTCTC CTCCTTGTAT AAATCCTGGT TGCTGTCTCT TTATGAGGAG TTGTGGCCCG
  181 TTGTCAGGCA ACGTGGCGTG GTGTGCACTG TGTTTGCTGA CGCAACCCCC ACTGGTTGGG
  241 GCATTGCCAC CACCTGTCAG CTCCTTTCCG GGACTTTCGC TTTCCCCCTC CCTATTGCCA
  301 CGGCGGAACT CATCGCCGCC TGCCTTGCCC GCTGCTGGAC AGGGGCTCGG CTGTTGGGCA
  961 CTGACAATTC CGTGGTGTTG TCGGGGAAGC TGACGTCCTT TCCATGGCTG CTCGCCTGTG
  421 TTGCCACCTG GATTCTGCGC GGGACGTCCT TCTGCTACGT CCCTTCGGCC CTCAATCCAG
  481 CGGACCTTCC TTCCCGCGGC CTGCTGCCGG CTCTGCGGCC TCTTCCGCGT CTTCGCCTTC
  541 GCCCTCAGAC GAGTCGGATC TCCCTTTGGG CCGCCTCCCC GC
SEQ ID No. 67:
bGH polyA+:
Bovine growth hormone poly A+ signal sequence (208 bp)
      1 CTGTGCCTTC TAGTTGCCAG CCATCTGTTG TTTGCCCCTC CCCCGTGCCT TCCTTGACCC
    61 TGGAAGGTGC CACTCCCACT GTCCTTTCCT AATAAAATGA GGAAATTGCA TCGCATTGTC
  121 TGAGTAGGTG TCATTCTATT CTGGGGGGTG GGGTGGGGCA GGACAGCAAG GGGGAGGATT
  181 GGGAAGACAA TAGCAGGCAT GCTGGGGA
SEQ ID No. 68:
SPA: Synthetic poly A+ (49 bp)
      1 AATAAAAGAT CTTTATTTTC ATTAGATCTG TGTGTTGGTT TTTTGTGTG
SEQ ID No. 69:
ppDyn full-length cDNA codon-optimized1 (765 bp)
      1 ATGGCTTGGC AGGGACTGGT GCTGGCTGCC TGCCTGCTGA TGTTCCCTAG CACCACCGCC
    61 GACTGCCTGA GCAGATGTAG CCTGTGCGCC GTGAAAACCC AGGACGGCCC CAAGCCCATC
  121 AACCCCCTGA TCTGCAGCCT GCAGTGTCAG GCCGCTCTGC TGCCTAGCGA GGAATGGGAG
  281 AGATGCCAGA GCTTCCTGAG CTTCTTCACC CCCAGCACCC TGGGCCTGAA CGACAAAGAG
  241 GACCTGGGCA GCAAGAGCGT GGGCGAGGGC CCTTATTCTG AGCTGGCCAA GCTGAGCGGC
  301 AGCTTTCTGA AAGAGCTGGA AAAGAGCAAG TTCCTGCCCA GCATCAGCAC CAAAGAGAAC
  361 ACCCTGAGCA AGAGCCTGGA AGAGAAGCTG CGGGGCCTGA GCGACGGCTT TAGAGAGGGC
  421 GCCGAGAGCG AGCTGATGAG GGACGCCCAG CTGAATGACG GCGCCATGGA AACCGGCACC
  481 CTGTACCTGG CCGAAGAGGA CCCCAAAGAA CAAGTGAAGA GATACGGCGG CTTCCTGCGG
S41 AAGTACCCCA AGAGATCTAG CGAGGTGGCC GGCGAAGGCG ACGGCGATTC TATGGGACAC
  601 GAGGACCTGT ACAAGCGCTA TGGCGGCTTT CTGAGGCGGA TCCGGCCCAA GCTGAAGTGG
  661 GACAACCAGA AACGCTACGG GGGATTCCTG AGGCGGCAGT TCAAGGTCGT GACCCGGTCC
  721 CAGGAAGATC CCAACGCCTA CAGCGGCGAG CTGTTCGACG CTTGA
SEQ ID No. 70:
ppDyn with shortened N-peptide codon-optimized2 (576 bp)
      1 ACCATGGCTT GGCAGGGACT TGTGCTTGCG GCTTGTCTCC TCATGTTCCC TAGTACAACT
    61 GCTGATTGCC TGAGCAGATG TAGCCTGTGC GCCGTGAAAA CCCAGGACGG CCCCAAGCCC
  121 ATCAACCCCC TGATCTGCAG CCTGCAGTGT CAGGCCGCTC TGCTGCCTAG CGAGGAATGG
  181 GAGAGATGCC AGAGCTTCCT GAGCTTCTTC ACCCCCAGCA CCCTGGGCCT GAACGACAAA
  241 GAGGACCTGG GCAGCAAGAG TGTAGGTGAA GGCCCTTACT CTGAGTTGGC AAAACTGTCT
  301 GGCAGTTTTC TGAGGAAGGA ACAGGTCAAG CGATATGGTG GCTTCCTGAG AAAGTATCCA
  361 AAGAGAAGCT CAGAAGTTGC AGGAGAAGGT GATGGGGATA GTATGGGACA CGAAGACCTG
  421 TACAAGCGCT ATGGAGGCTT TCTGAGGAGA ATACGTCCCA AACTGAAATG GGACAACCAG
  481 AAACGATATG GAGGCTTCTT GAGAAGGCAG TTCAAAGTGG TGACTAGAAG CCAAGAGGAC
  541 CCTAATGCCT ACTCAGGGGA GTTGTTTGAT GCCTGA
SEQ ID No. 71:
ppDyn with shortened N-peptide codon-optimized2 (399 bp)
      1 ATGGCTTGGC AGGGACTTGT GCTTGCGGCT TGTCTCCTCA TGTTCCCTAG TACAACTGCT
    61 AAAGAGGACC TGGGCAGCAA GAGTGTAGGT GAAGGCCCTT ACTCTGAGTT GGCAAAACTG
  121 TCTGGCAGTT TTCTGAGGAA GGAACAGGTC AAGCGATATG GTGGCTTCCT GAGAAAGTAT
  181 CCAAAGAGAA GCTCAGAAGT TGCAGGAGAA GGTGATGGGG ATAGTATGGG ACACGAAGAC
  241 CTGTACAAGC GCTATGGAGG CTTTCTGAGG AGAATACGTC CCAAACTGAA ATGGGACAAC
  301 CAGAAACGAT ATGGAGGCTT CTTGAGAAGG CAGTTCAAAG TGGTGACTAG AAGCCAAGAG
  361 GACCCTAATG CCTACTCAGG GGAGTTGTTT GATGCCTGA
SEQ ID No. 72:
ppDyn with shortened N-peptide codon-optimized2 (519 bp)
      1 ATGGCTTGGC AGGGACTTGT GCTTGCGGCT TGTCTCCTCA TGTTCCCTAG TACAACTGCT
    61 GATTGCCTGA GCAGATGTAG CCTGTGCGCC GTGAAAACCC AGGACGGCCC CAAGCCCATC
  221 AACCCCCTGA TCTGCAGCCT GCAGTGTCAG GCCGCTCTGC TGCCTAGCGA GGAATGGGAG
  181 AGATGCCAGA GCTTCCTGAG CTTCTTCACC CCCAGCACCC TGGGCCTGAA CGACAAAGAG
  241 TCTGGCAGTT TTCTGAGGAA GGAACAGGTC AAGCGATATG GTGGCTTCCT GAGAAAGTAT
  301 CCAAAGAGAA GCTCAGAAGT TGCAGGAGAA GGTGATGGGG ATAGTATGGG ACACGAAGAC
  361 CTGTACAAGC GCTATGGAGG CTTTCTGAGG AGAATACGTC CCAAACTGAA ATGGGACAAC
  421 CAGAAACGAT ATGGAGGCTT CTTGAGAAGG CAGTTCAAAG TGGTGACTAG AAGCCAAGAG
  481 GACCCTAATG CCTACTCAGG GGAGTTGTTT GATGCCTGA
SEQ ID No. 73:
ppDyn with N-terminus from POMC fused to C-terminal part of pDyn
codon-optimized2 (417 bp)
      1 ATGCCAAGAT CATGCTGTTC ACGTAGTGGA GCCCTTCTCT TGGCACTTCT TCTGCAAGCA
    61 AGCATGGAAG TCAGAGGTTG GTGTTTGGAG AGTTCCCAAT GCCAAGACCT GACTACTGAG
  121 TCAAACCTGC TGGAATGCAT AAGAGCCTGT AAACCCAAGG AACAGGTGAA ACGCTATGGT
  181 GGGTTTCTCA GGAAATATCC CAAAAGATCC TCTGAGGTTG CTGGTGAAGG TGATGGGGAC
  241 AGTATGGGCC ATGAGGACCT GTACAAGAGA TATGGTGGAT TCCTCAGAAG AATCAGACCC
  301 AAACTGAAGT GGGACAACCA GAAGAGATAT GGAGGGTTTC TGAGGAGACA GTTCAAGGTA
  361 GTCACCAGAT CCCAAGAGGA CCCTAACGCG TATAGTGGGG AACTCTTTGA TGCATGA
SEQ ID No. 74:
SSAAV-pDyn (2820 bp)
      1 TTGGCCACTC CCTCTCTGCG CGCTCGCTCG CTCACTGAGG CCGGGCGACC AAAGGTCGCC
    61 CGACGCCCGG GCTTTGCCCG GGCGGCCTCA GTGAGCGAGC GAGCGCGCAG AGAGGGAGTG
  121 GCCAACTCCA TCACTAGGGG TTCCTAATTC GATATCAAGC TTATCGATAC CGTCGACCTC
  181 GAGGGGGTAA TAGTAATCAA TTACGGGGTC ATTAGTTCAT AGCCCATATA TGGAGTTCCG
  241 CGTTACATAA CTTACGGTAA ATGGCCCGCC TGGCTGACCG CCCAACGACC CCCGCCCATT
  301 GACGTCAATA ATGACGTATG TTCCCATAGT AACGCCAATA GGGACTTTCC ATTGACGTCA
  361 ATGGGTGGAG TATTTACGGT AAACTGCCCA CTTGGCAGTA CATCAAGTGT ATCATATGCC
  421 AAGTACGCCC CCTATTGACG TCAATGACGG TAAATGGCCC GCCTGGCATT ATGCCCAGTA
  483 CATGACCTTA TGGGACTTTC CTACTTGGCA GTACATCTAC GTATTAGTCA TCGCTATTAC
  541 CATGGTCGAG GTGAGCCCCA CGTTCTGCTT CACTCTCCCC ATCTCCCCCC CCTCCCCACC
  601 CCCAATTTTG TATTTATTTA TTTTTTAATT ATTTTGTGCA GCGATGGGGG CCGGGGGGGG
  661 GGGGGGGCGC GCGCCAGGCG GGGGGGGGCG GGGCGAGGGG CGGGGCGGGG CGAGGCGGAA
  721 AGGTGCGGCG GCAGCCAATC AGAGCGGCGC GCTCCGAAAG TTTCCTTTTA TGGCGAGGCG
  781 GCGGCGGCCG CGGCCCTATA AAAAGCGAAG CGCGCGGCGG GCGGGAGTCG CTGCGCGCTG
  841 CCTTCGCCCC GTGCCCCGCT CCGCCGCCGC CTCGCGCCGC CCGCCCCGGC TCTGACTGAC
  901 CGCGTTACTC CCACAGGTGA GCGGGCGGGA CGGCCCTTCT CCTCCGGGCT GTAATTAGCG
  961 CTTGGTTTAA TGACGGCTTG TTTCTTTTCT GTGGCTGCGT GAAAGCCTTA AAGGGCTCCG
1021 GGAGGGCCCT AGAGCCTCTG CTAACCATGT TCATGCCTTC TTCTTTTTCC TACAGCGGCC
1881 GCGCCACCAT GGCTTGGCAG GGACTGGTGC TGGCTGCCTG CCTGCTGATG TTCCCTAGCA
1142 CCACCGCCGA CTGCCTGAGC AGATGTAGCC TGTGCGCCGT GAAAACCCAG GACGGCCCCA
1201 AGCCCATCAA CCCCCTGATC TGCAGCCTGC AGTGTCAGGC CGCTCTGCTG CCTAGCGAGG
1261 AATGGGAGAG ATGCCAGAGC TTCCTGAGCT TCTTCACCCC CAGCACCCTG GCCCTGAACG
1321 ACAAAGAGGA CCTGGGCAGC AAGAGCGTGG GCGAGGGCCC TTATTCTGAG CTGGCCAAGC
1381 TGAGCGGCAG CTTTCTGAAA GAGCTGGAAA AGAGCAAGTT CCTGCCCAGC ATCAGCACCA
1442 AAGAGAACAC CCTGAGCAAG AGCCTGGAAG AGAAGCTCCG GGGCCTGAGC GACGGCTTTA
1501 GAGAGGGCGC CGAGAGCGAG CTGATGAGGG ACGCCCAGCT GAATGACGGC GCCATGGAAA
1562 CCGGCACCCT GTACCTGGCC GAAGAGGACC CCAAAGAACA AGTGAAGAGA TACGGCGGCT
1621 TCCTGCGGAA GTACCCCAAG AGATCTAGCG AGGTGGCCGG CGAAGGCGAC GGCGATTCTA
1681 TGGGACACGA GGACCTGTAC AAGCGCTATG GCGGCTTTCT GAGGCGGATC CGGCCCAAGC
1741 TGAAGTGGGA CAACCAGAAA CGCTACGGGG GATTCCTGAG GCGGCAGTTC AAGGTCGTGA
1801 CCCGGTCCCA GGAAGATCCC AACGCCTACA GCGGCGAGCT GTTCGACGCT TGAGTCGACC
1861 TCTGGATTAC AAAATTTGTG AAAGATTGAC TGGTATTCTT AACTATGTTG CTCCTTTTAC
1921 GCTATGTGGA TACGCTGCTT TAATGCCTTT GTATCATGCT ATTGCTTCCC GTATGGCTTT
1981 CATTTTCTCC TCCTTGTATA AATCCTGGTT GCTGTCTCTT TATGAGGAGT TGTGGCCCGT
2241 TGTCAGGCAA CGTGGCGTGG TGTGCACTGT GTTTGCTGAC GCAACCCCCA CTGGTTGGGG
2101 CATTGCCACC ACCTGTCAGC TCCTTTCCGG GACTTTCGCT TTCCCCCTCC CTATTGCCAC
2161 GGCGGAACTC ATCGCCGCCT GCCTTGCCCG CTGCTGGACA GGGGCTCGGC TGTTGGGCAC
2221 TGACAATTCC GTGGTGTTGT CGGGGAAGCT GACGTCCTTT CCATGGCTGC TCGCCTGTGT
2281 TGCCACCTGG ATTCTGCGCG GGACGTCCTT CTGCTACGTC CCTTCGGCCC TCAATCCAGC
2341 GGACCTTCCT TCCCGCGGCC TGCTGCCGGC TCTGCGGCCT CTTCCGCGTC TTCGCCTTCG
2401 CCCTCAGACG AGTCGGATCT CCCTTTGGGC CGCCTCCCCG CCTGATCAGC CTCGACTGTG
2462 CCTTCTAGTT GCCAGCCATC TGTTGTTTGC CCCTCCCCCG TGCCTTCCTT GACCCTGGAA
2521 GGTGCCACTC CCACTGTCCT TTCCTAATAA AATGAGGAAA TTGCATCGCA TTGTCTGAGT
2581 AGGTGTCATT CTATTCTGGG GGGTGGGGTG GGGCAGGACA GCAAGGGGGA GGATTGGGAA
2641 GACAATAGCA GGCATGCTGG GGAGAGAAGC TTACAAGGAA CCCCTAGTGA TCGAGTTGGC
2701 CACTCCCTCT CTGCGCGCTC GCTCGCTCAC TGAGGCCGGG CGACCAAAGG TCGCCCGACG
2761 CCCGGGCTTT GCCCGGGCGG CCTCAGTGAG CGAGCGAGCG CGCAGAGAGG GAGTGGCCAA
SEQ ID No. 75:
scAAV-pDyn (2210 bp)
      1 CCACTCCCTC TCTGCGCGCT CGCTCGCTCA CTGAGGCCGG GCGACCAAAG GTCGCCCGAC
    61 GCCCGGGCTT TGCCCGGGCG GCCTCAGTGA GCGAGCGAGC GCGCAGAGAG GGAGTGGAAT
  221 TCGATATCAA GCTTATCGAT ACCGTCGACC TCGAGGGGGT AATAGTAATC AATTACGGGG
  181 TCATTAGTTC ATAGCCCATA TATGGAGTTC CGCGTTACAT AACTTACGGT AAATGGCCCG
  241 CCTGGCTGAC CGCCCAACGA CCCCCGCCCA TTGACGTCAA TAATGACGTA TGTTCCCATA
  301 GTAACGCCAA TAGGGACTTT CCATTGACGT CAATGGGTGG AGTATTTACG GTAAACTGCC
  361 CACTTGGCAG TACATCAAGT GTATCATATG CCAAGTACGC CCCCTATTGA CGTCAATGAC
  421 GGTAAATGGC CCGCCTGGCA TTATGCCCAG TACATGACCT TATGGGACTT TCCTACTTGG
  481 CAGTACATCT ACGTATTAGT CATCGCTATT ACCATGGTCG AGGTGAGCCC CACGTTCTGC
  541 TTCACTCTCC CCATCTCCCC CCCCTCCCCA CCCCCAATTT TGTATTTATT TATTTTTTAA
  621 TTATTTTGTG CAGCGATGGG GGCGGGGGGG GGGGGGGGGC GCGCGCCAGG CGGGGGGGGG
  661 CGGGGCGAGG GGCGGGGCGG GGCGAGGCGG AGAGGTGCGG CGGCAGCCAA TCAGAGCGGC
  721 GCGCTCCGAA AGTTTCCTTT TATGGCGAGG CGGCGGCGGC GGCGGCCCTA TAAAAAGCGA
  781 AGCGCGCGGC GGGCGGGAGT CGCTGCGCGC TGCCTTCGCC CCGTGCCCCG CTCCGCCGCC
  841 GCCTCGCGCC GCCCGCCCCG GCTCTGACTG ACCGCGTTAC TCCCACAGGT GAGCGGGGGG
  901 GACGGCCCTT CTCCTCCGGG CTGTAATTAG CGCTTGGTTT AATGACGGCT TGTTTCTTTT
  961 CTGTGGCTGC GTGAAAGCCT TGAGGGGCTC CGGGAGGGCC CTAGAGCCTC TGCTAACCAT
1021 GTTCATGCCT TCTTCTTTTT CCTACAGCGG CCGCGCCACC ATGGCTTGGC AGGGACTGGT
1081 GCTGGCTGCC TGCCTGCTGA TGTTCCCTAG CACCACCGCC GACTGCCTGA GCAGATGTAG
1141 CCTGTGCGCC GTGAAAACCC AGGACGGCCC CAAGCCCATC AACCCCCTGA TCTGCAGCCT
1201 GCAGTGTCAG GCCGCTCTGC TGCCTAGCGA GGAATGGGAG AGATGCCAGA GCTTCCTGAG
1261 CTTCTTCACC CCCAGCACCC TGGGCCTGAA CGACAAAGAG GACCTGGGCA GCAAGAGCGT
1321 GGGCGAGGGC CCTTATTCTG AGCTGGCCAA GCTGAGCGGC AGCTTTCTGA AAGAGCTGGA
1381 AAAGAGCAAG TTCCTGCCCA GCATCAGCAC CAAAGAGAAC ACCCTGAGCA AGAGCCTCGA
1441 AGAGAAGCTG CGGGGCCTGA GCGACGGCTT TAGAGAGGGC GCCGAGAGCG AGCTGATGAG
1501 GGACGCCCAG CTGAATGACG GCGCCATGGA AACCGGCACC CTGTACCTGG CCGAAGAGGA
3561 CCCCAAAGAA CAAGTGAAGA GATACGGCGG CTTCCTGCGG AAGTACCCCA AGAGATCTAG
1621 CGAGGTGGCC GGCGAAGGCG ACGGCGATTC TATGGGACAC GAGGACCTGT ACAAGCGCTA
1681 TGGCGGCTTT CTGAGGCGGA TCCGGCCCAA GCTGAAGTGG GACAACCAGA AACGCTACGG
1741 GGGATTCCTG AGGCGGCAGT TCAAGGTCGT GACCCGGTCC CAGGAAGATC CCAACGCCTA
1801 CAGCGGCGAG CTGTTCGACG CTTGAGTCGA CTAGAGCTCG CTGATCAGCC TCGACTGTGC
1861 CTTCTAGTTG CCAGCCATCT GTTGTTTGCC CCTCCCCCGT GCCTTCCTTG ACCCTGGAAG
1921 GTGCCACTCC CACTGTCCTT TCCTAATAAA ATGAGGAAAT TGCATCGCAT TGTCTGAGTA
1981 GGTGTCATTC TATTCTGGGG GGTGGGGTGG GGCAGGACAG CAAGGGGGAG GATTGGGAAG
2041 ACAATAGCAG GCATGCTGGG GAGAGATCGA TCTAGGAACC CCTAGTGATG GAGTTGGCCA
2101 CTCCCTCTCT GCGCGCTCGC TCGCTCACTG AGGCCGCCCG GGCAAAGCCC GGGCGTCGGG
2161 CGACCTTTGG TCGCCCGGCC TCAGTGAGCG AGCGAGCGCG CAGAGAGGGA GTGGCCAAC
SEQ ID No. 76:
scAAV-pDyn (2603 bp)
      1 TTGGCCACTC CCTCTCTGCG CGCTCGCTCG CTCACTGAGG CCGGGCGACC AAAGGTCGCC
    61 CGACGCCCGG GCTTIGCCCG GGCGGCCTCA GTGAGCGAGC GAGCGCGCAG AGAGGGAGTG
  121 GATTCGATAT CAAGCTTATC GATACCGTCG ACCTCGAGGG GGTAATAGTA ATCAATTACG
  281 GGGTCATTAG TTCATAGCCC ATATATGGAG TTCCGCGTTA CATAACTTAC GGTAAATGGC
  241 CCGCCTGGCT GACCGCCCAA CGACCCCCGC CCATTGACGT CAATAATGAC GTATGTTCCC
  301 ATAGTAACGC CAATAGGGAC TTTCCATTGA CGTCAATGGG TGGAGTATTT ACGGTAAACT
  361 GCCCACTTGG CAGTACATCA AGTGTATCAT ATGCCAAGTA CGCCCCCTAT TGACGTCAAT
  421 GACGGTAAAT GGCCCGCCTG GCATTATGCC CAGTACATGA CCTTATGGGA CTTTCCTACT
  481 TGGCAGTACA TCTACGTATT AGTCATCGCT ATTACCATGG TCGAGGTGAG CCCCACGTTC
  541 TGCTTCACTC TCCCCATCTC CCCCCCCTCC CCACCCCCAA TTTTGTATTT ATTTATTTTT
  601 TAATTATTTT GTGCAGCGAT GGGGGCGGGG GGGGGGGGGG GGCGCGCGCC AGGCGGGGCG
  661 GGGGGGGGCG AGGGGGGGGG CGGGGCGAGG CGGAGAGGTG CGGCGGCAGC CAATCAGAGC
  721 GGCGCGCTCC GAAAGTTTCC TTTTATGGCG AGGCGGCGGC GGCGGCGGCC CTATAAAAAG
  781 CGAAGCGCGC GGCGGGGGGG AGTCGCTGCG CGCTGCCTTC GCCCCGTGCC CCGCTCCGCC
  841 GCCGCCTCGC GCCGCCCGCC CCGGCTCTGA CTGACCGCGT TACTCCCACA GGTGAGCGGG
  901 CGGGACGGCC CTTCTCCTCC GGGCTGTAAT TAGCGCTTGG TTTAATGACG GCTTGTTTCT
  961 TTTCTGTGGC TGCGTGAAAG CCTTGAGGGG CTCCGGGAGG GCCCTAGAGC CTCTGCTAAC
2021 CATGTTCATG CCTTCTTCTT TTTCCTACAG CGGCCGCGCC ACCATGGCTT GGCAGGGACT
1081 TGTGCTTGCG GCTTGTCTCC TCATGTTCCC TAGTACAACT GCTGATTGCC TGAGCAGATG
1141 TAGCCTGTGC GCCGTGAAAA CCCAGGACGG CCCCAAGCCC ATCAACCCCC TGATCTGCAG
1201 CCTGCAGTGT CAGGCCGCTC TGCTGCCTAG CGAGGAATGG GAGAGATGCC AGAGCTTCCT
1261 GAGCTTCTTC ACCCCCAGCA CCCTGGGCCT GAACGACAAA GAGGACCTGG GCAGCAAGAG
1321 TGTAGGTGAA GGCCCTTACT CTGAGTTGGC AAAACTGTCT GGCAGTTTTC TGAGGAAGGA
1381 ACAGGTCAAG CGATATGGTG GCTTCCTGAG AAAGTATCCA AAGAGAAGCT CAGAAGTTGC
1441 AGGAGAAGGT GATGGGGATA GTATGGGACA CGAAGACCTG TACAAGCGCT ATGGAGGCTT
1501 TCTGAGGAGA ATACGTCCCA AACTGAAATG GGACAACCAG AAACGATATG GAGGCTTCTT
1561 GAGAAGGCAG TTCAAAGTGG TGACTAGAAG CCAAGAGGAC CCTAATGCCT ACTCAGGGGA
1621 GTTGTTTGAT GCCTGAGTCG ACCTCTGGAT TACAAAATTT GTGAAAGATT GACTGGTATT
1681 CTTAACTATG TIGCTCCTTT TACGCTATGT GGATACGCTG CTTTAATGCC TTTGTATCAT
1741 GCTATTGCTT CCCGTATGGC TTTCATTTTC TCCTCCTTGT ATAAATCCTG GTTGCTGTCT
1801 CTTTATGAGG AGTIGTGGCC CGTTGTCAGG CAACGTCGCG TGGTGTGCAC TGTGTTTGCT
2861 GACGCAACCC CCACTGGTTG GGGCATTGCC ACCACCTGTC AGCTCCTTTC CGGGACTTTC
1921 GCTTTCCCCC TCCCTATTGC CACGGCGGAA CTCATCGCCG CCTGCCTTGC CCGCTGCTGG
1981 ACAGGGGCTC GGCTGTTGGG CACTGACAAT TCCGTGGTGT TGTCGGGGAA GCTGACGTCC
2041 TTTCCATGGC TGCTCGCCTG TGTTGCCACC TGGATTCTGC GCGGGACGTC CTTCTGCTAC
2101 GTCCCTTCGG CCCTCAATCC AGCGGACCTT CCTTCCCGCG GCCTGCTGCC GGCTCTGCGG
2161 CCTCTTCCGC GTCTTCGCCT TCGCCCTCAG ACGAGTCGGA TCTCCCTTTG GGCCGCCTCC
2221 CCGCCTGATC AGCCTCGACT GTGCCTTCTA GTTGCCAGCC ATCTGTTGTT TGCCCCTCCC
2281 CCGTGCCTTC CTTGACCCTG GAAGGTGCCA CTCCCACTGT CCTTTCCTAA TAAAATGAGG
2341 AAATTGCATC GCATTGTCTG AGTAGGTGTC ATTCTATTCT GGGGGGTGGG GTGGGGCAGG
2401 ACAGCAAGGG GGAGGATTGG GAAGACAATA GCAGGCATGC TGGGGAGAGA AGCTTACAAG
2461 GAACCCCTAG TGATGGAGTT GGCCACTCCC TCTCTGCGCG CTCGCTCGCT CACTGAGGCC
2521 GGGCGACCAA AGGTCGCCCG ACGCCCGGGC TTTGCCCGGG CGGCCTCAGT GAGCGAGCGA
2581 GCGCGCAGAG AGGGAGTGGC CAA
SEQ ID No. 77:
scAAV-syn-pDyn (2164 bp)
      1 TTGGCCACTC CCTCTCTGCG CGCTCGCTCG CTCACTGAGG CCGGGCGACC AAAGGTCGCC
    61 CGACGCCCGG GCTTTGCCCG GGCGGCCTCA GTGAGCGAGC GAGCGCGCAG AGAGGGAGTG
  121 GATTCGATAT CAAGCTTATC GATACCGTCG ACCTCGAGGG GGAGTGCAAG TGGGTTTTAG
  181 GACCAGGATG AGGCGGGGTG GGGGTGCCTA CCTGACGACC GACCCCGACC CACTGGACAA
  241 GCACCCAACC CCCATTCCCC AAATTGCGCA TCCCCTATCA GAGAGGGGGA GGGGAAACAG
  301 GATGCGGCGA GGCGCGTGCG CACTGCCAGC TTCAGCACCG CGGACAGTGC CTTCGCCCCC
  361 GCCTGGCGGC GCGCGCCACC GCCGCCTCAG CACTGAAGGC GCGCTGACGT CACTCGCCGG
  421 TCCCCCGCAA ACTCCCCTTC CCGGCCACCT TGGTCGCGTC CGCGCCGCCG CCGGCCCAGC
  481 CGGACCGCAC CACGCGAGGC GCGAGATAGG GGGGCACGGG CGCGACCATC TGCGCTGCGG
  541 CGCCGGCGAC TCAGCGCTGC CTCAGTCTGC GGTGGGCAGC GGAGGAGTCG TGTCGTGCCT
  601 GAGAGCGCAG GGGCCGCGC CACCATGGCT TGGCAGGGAC TTGTGCTTGC GGCTTGTCTC
  661 CTCATGTTCC CTAGTACAAC TGCTGATTGC CTGAGCAGAT GTAGCCTGTG CGCCGTGAAA
  721 ACCCAGGACG GCCCCAAGCC CATCAACCCC CTGATCTGCA GCCTGCAGTG TCAGGCCGCT
  781 CTGCTGCCTA GCGAGGAATG GGAGAGATGC CAGAGCTTCC TGAGCTTCTT CACCCCCAGC
  841 ACCCTGGGCC TGAACGACAA AGAGGACCTG GGCAGCAAGA GTGTAGGTGA AGGCCCTTAC
  901 TCTGAGTTGG CAAAACTGTC TGGCAGTTTT CTGAGGAAGG AACAGGTCAA GCGATATGGT
  961 GGCTTCCTGA GAAAGTATCC AAAGAGAAGC TCAGAAGTTG CAGGAGAAGG TGATGGGGAT
1021 AGTATGGGAC ACGAAGACCT GTACAAGCGC TATGGAGGCT TTCTGAGGAG AATACGTCCC
1081 AAACTGAAAT GGGACAACCA GAAACGATAT GGAGGCTTCT TGAGAAGGCA GTTCAAAGTG
1141 GTGACTAGAA GCCAAGAGGA CCCTAATGCC TACTCAGGGG AGTTGTTTGA TGCCTGAGTC
1201 GACCTCTGGA TTACAAAATT TGTGAAAGAT TGACTGGTAT TCTTAACTAT GTTGCTCCTT
1261 TTACGCTATG TGGATACGCT GCTTTAATGC CTTTGTATCA TGCTATTGCT TCCCGTATGG
1321 CTTTCATTTT CTCCTCCTTG TATAAATCCT GGTTGCTGTC TCTTTATGAG GAGTTGTGGC
13A1 CCGTTGTCAG GCAACGTGGC GTGGTGTGCA CTGTGTTTGC TGACGCAACC CCCACTGGTT
1441 GGGGCATTGC CACCACCTGT CAGCTCCTTT CCGGGACTTT CGCTTTCCCC CTCCCTATTG
1501 CCACGGCGGA ACTCATCGCC GCCTGCCTTG CCCGCTGCTG GACAGGGGCT CGGCTGTTGG
1561 GCACTGACAA TTCCGTGGTG TTGTCGGGGA AGCTGACGTC CTTTCCATGG CTGCTCGCCT
1621 GTGTTGCCAC CTGGATTCTG CGCGGGACGT CCTTCTGCTA CGTCCCTTCG GCCCTCAATC
1681 CAGCGGACCT TCCTTCCCGC GGCCTGCTGC CGGCTCTGCG GCCTCTTCCG CGTCTTCGCC
1741 TTCGCCCTCA GACGAGTCGG ATCTCCCTTT GGGCCGCCTC CCCGCCTGAT CAGCCTCGAC
1801 TGTGCCTTCT AGTTGCCAGC CATCTGTTGT TTGCCCCTCC CCCGTGCCTT CCTTGACCCT
1861 GGAAGGTGCC ACTCCCACTG TCCTTTCCTA ATAAAATGAG GAAATTGCAT CGCATTGTCT
1021 GAGTAGGTGT CATTCTATTC TGGGGGGTGG GGTGGGGCAG GACAGCAAGG GGGAGGATTG
1981 GGAAGACAAT AGCAGGCATG CTGGGGAGAG AAGCTTACAA GGAACCCCTA GTGATGGAGT
2041 TGGCCACTCC CTCTCTGCGC GCTCGCTCGC TCACTGAGGC CGGGCGACCA AAGGTCGCCC
2101 GACGCCCGGG CTTTGCCCGG GCGGCCTCAG TGAGCGAGCG AGCGCGCAGA GAGGGAGTGG
2161 CCAA
SEQ ID No. 78:
scAAV-pDyn (2388 bp)
      1 TTGGCCACTC CCTCTCTGCG CGCTCGCTCG CTCACTGAGG CCGGGCGACC AAAGGTCGCC
    61 CGACGCCCGG GCTTTGCCCG GGCGGCCTCA GTGAGCGAGC GAGCGCGCAG AGAGGGAGTG
  121 GATTCGATAT CATCGATACC GTCCATATAT GGAGTTCCGC GTTACATAAC TTACGGTAAA
  181 TGGCCCGCCT GGCTGACCGC CCAACGACCC CCGCCCATTG ACGTCAATAA TGACGTATGT
  241 TCCCATAGTA ACGCCAATAG GGACTTTCCA TTGACGTCAA TGGGTGGAGT ATTTACGGTA
  301 AACTGCCCAC TTGGCAGTAC ATCAAGTGTA TCATATGCCA AGTACGCCCC CTATTGACGT
  361 CAATGACGGT AAATGGCCCG CCTGGCATTA TGCCCAGTAC ATGACCTTAT GGGACTTTCC
  421 TACTTGGCAG TACATCTACG TATTAGTCAT CGCTATTACC ATGGTCGAGG TGAGCCCCAC
  481 GTTCTGCTTC ACTCTCCCCA TCTCCCCCCC CTCCCCACCC CCAATTTTGT ATTTATTTAT
  541 TTTTTAATTA TTTTGTGCAG CGATGGGGGC GGGGGGGGGG GGGGGGCGCG CGCCAGGCGG
  601 GGCGGGGCGG GGCGAGGGGC GGGGGGGGGC GAGGCGGAGA GGTGCGGCGG CAGCCAATCA
  661 GAGCGGCGCG CTCCGAAAGT TTCCTTTTAT GGCGAGGCGG CGGCGGCGGC GGCCCTATAA
  721 AAAGCGAAGC GCGCGGGGG CGGGAGTCGC TGCGCGCTGC CTTCGCCCCG TGCCCCGCTC
  781 CGCCGCCGCC TCGCGCCGCC CGCCCCGGCT CTGACTGACC GCGTTACTCC CACAGGTGAG
  841 CGGGCGGGAC GGCCCTTCTC CTCCGGGCTG TAATTAGCGC TTGGTTTAAT GACGGCTTGT
  901 TTCTTTTCTG TGGCTGCGTG AAAGCCTTGA GGGGCTCCGG GAGGGCCCTA GAGCCTCTGC
  061 TAACCATGTT CATGCCTTCT TCTTTTTCCT ACAGCGGCCG CGCCACCATG GCTTGGCAGG
1021 GACTTGTGCT TGCGGCTTGT CTCCTCATGT TCCCTAGTAC AACTGCTGAT TGCCTGAGCA
1881 GATGTAGCCT GTGCGCCGTG AAAACCCAGG ACGGCCCCAA GCCCATCAAC CCCCTGATCT
1241 GCAGCCTGCA GTGTCAGGCC GCTCTGCTGC CTAGCGAGGA ATGGGAGAGA TGCCAGAGCT
1201 TCCTGAGCTT CTTCACCCCC AGCACCCTGG GCCTGAACGA CAAAGAGGAC CTGGGCAGCA
1261 AGAGTGTAGG TGAAGGCCCT TACTCTGAGT TGGCAMAACT GTCTGGCAGT TTTCTGAGGA
1321 AGGAACAGGT CAAGCGATAT GGTGGCTTCC TGAGAAAGTA TCCAAAGAGA AGCTCAGAAG
1381 TTGCAGGAGA AGGTGATGGG GATAGTATGG GACACGAAGA CCTGTACAAG CGCTATGGAG
1441 GCTTTCTGAG GAGAATACGT CCCAAACTGA AATGGGACAA CCAGAAACGA TATGGAGGCT
1501 TCTTGAGAAG GCAGTTCAAA GTGGTGACTA GAAGCCAAGA GGACCCTAAT GCCTACTCAG
1561 GGGAGTTGTT TGATGCCTGA GTCGACCTCT GGATTACAAA ATTTGTGAAA GATTGACTGG
1621 TATTCTTAAC TATGTTGCTC CTTTTACGCT ATGTGGATAC GCTGCTTTAA TGCCTTTGTA
1681 TCATGCTATT GCTTCCCGTA TGGCTTTCAT TTTCTCCTCC TTGTATAAAT CCTGGTTGCT
1741 GTCTCTTTAT GAGGAGTTGT GGCCCGTTGT CAGGCAACGT GGCGTGGTGT GCACTGTGTT
1801 TGCTGACGCA ACCCCCACTG GTTGGGGCAT TGCCACCACC TGTCAGCTCC TTTCCGGGAC
1861 TTTCGCTTTC CCCCTCCCTA TTGCCACGGC GGAACTCATC GCCGCCTGCC TTGCCCGCTG
1921 CTGGACAGGG GCTCGGCTGT TGGGCACTGA CAATTCCGTG GTGTTGTCGG GGAAGCTGAC
1981 GTCCTTTCCA TGGCTGCTCG CCTGTGTTGC CACCTGGATT CTGCGCGGGA CGTCCTTCTG
2041 CTACGTCCCT TCGGCCCTCA ATCCAGCGGA CCTTCCTTCC CGCGGCCTGC TGCCGGCTCT
2101 GCGGCCTCTT CCGCGTCTTC GCCTTCGCCC TCAGACGAGT CGGATCTCCC TTTGGGCCGC
2161 CTCCCCGCCT GATCAGCCTC GAAATAAAAG ATCTTTATTT TCATTAGATC TGTGTGTTGG
2221 TTTTTTGTGT GGAGAAGCTT ACAAGGAACC CCTAGTGATG GAGTTGGCCA CTCCCTCTCT
2281 GCGCGCTCGC TCGCTCACTG AGGCCGGGCG ACCAAAGGTC GCCCGACGCC CGGGCTTTGC
2341 CCGGGCGGCC TCAGTGAGCG AGCGAGCGCG CAGAGAGGGA GTGGCCAA
SEQ ID No. 79:
scAAV-pDyn (2270 bp)
      1 TTGGCCACTC CCTCTCTGCG CGCTCGCTCG CTCACTGAGG CCGGGCGACC AAAGGTCGCC
    61 CGACGCCCGG GCTTTGCCCG GGCGGCCTCA GTGAGCGAGC GAGCGCGCAG AGAGGGAGTG
  121 GATTCGATAT CAAGCTTATC GATACCGTCG ACCTCGAGGG GGTAATAGTA ATCAATTACG
  181 GGGTCATTAG TTCATAGCCC ATATATGGAG TTCCGCGTTA CATAACTTAC GGTAAATGGC
  241 CCGCCTGGCT GACCGCCCAA CGACCCCCGC CCATTGACGT CAATAATGAC GTATGTTCCC
  301 ATAGTAACGC CAATAGGGAC TTTCCATTGA CGTCAATGGG TGGAGTATTT ACGGTAAACT
  361 GCCCACTTGG CAGTACATCA AGTGTATCAT ATGCCAAGTA CGCCCCCTAT TGACGTCAAT
  421 GACGGTAAAT GGCCCGCCTG GCATTATGCC CAGTACATGA CCTTATGGGA CTTTCCTACT
  481 TGGCAGTACA TCTACGTATT AGTCATCGCT ATTACCATGG TCGAGGTGAG CCCCACGTTC
  541 TGCTTCACTC TCCCCATCTC CCCCCCCTCC CCACCCCCAA TTTTGTATTT ATTTATTTTT
  601 TAATTATTTT GTGCAGCGAT GGGGGCGGGG GGGGGGGGGG GGCGCGCGCC AGGCGGGGCG
  661 GGGGGGGGCG AGGGGCGGGG CGGGGCGAGG CGGAGAGGTG CGGCGGCAGC CAATCAGAGC
  221 GGCGCGCTCC GAAAGTTTCC TTTTATGGCG AGGCGGCGGC GGCGGCGGCC CTATAAAAAG
  781 CGAAGCGCGC GGCGGGCGGG AGTCGCTGCG CGCTGCCTTC GCCCCGTGCC CCGCTCCGCC
  841 GCCGCCTCGC GCCGCCCGCC CCGGCTCTGA CTGACCGCGT TACTCCCACA GGTGAGCGGG
  901 CGGGACGGCC CTTCTCCTCC GGGCTGTAAT TAGCGCTTGG TTTAATGACG GCTTGTTTCT
  961 TTTCTGTGGC TGCGTGAAAG CCTTGAGGGG CTCCGGGAGG GCCCTAGAGC CTCTGCTAAC
1021 CATGTTCATG CCTTCTTCTT TTTCCTACAG CGGCCGCGCC ACCATGGCTT GGCAGGGACT
1881 TGTGCTTGCG GCTTGTCTCC TCATGTTCCC TAGTACAACT GCTAAAGAGG ACCTGGGCAG
1341 CAAGAGTGTA GGTGAAGGCC CTTACTCTGA GTTGGCAAAA CTGTCTGGCA GTTTTCTGAG
1201 GAAGGAACAG GTCAAGCGAT ATGGTGGCTT CCTGAGAAAG TATCCAAAGA GAAGCTCAGA
1262 AGTTGCAGGA GAAGGTGATG GGGATAGTAT GGGACACGAA GACCTGTACA AGCGCTATGG
1321 AGGCTTTCTG AGGAGAATAC GTCCCAAACT GAAATGGGAC AACCAGAAAC GATATGGAGG
1381 CTTCTTGAGA AGGCAGTTCA AAGTGGTGAC TAGAAGCCAA GAGGACCCTA ATGCCTACTC
1441 AGGGGAGTTG TTTGATGCCT GAGTCGACCT CTGGATTACA AAATTTGTGA AAGATTGACT
1501 GGTATTCTTA ACTATGTTGC TCCTTTTACG CTATGTGGAT ACGCTGCTTT AATGCCTTTG
1561 TATCATGCTA TTGCTTCCCG TATGGCTTTC ATTTTCTCCT CCTTGTATAA ATCCTGGTTG
1621 CTGTCTCTTT ATGAGGAGTT GTGGCCCGTT GTCAGGCAAC GTGGCGTGGT GTGCACTGTG
1681 TTTGCTGACG CAACCCCCAC TGGTTGGGGC ATTGCCACCA CCTGTCAGCT CCTTTCCGGG
1743 ACTTTCGCTT TCCCCCTCCC TATTGCCACG GCGGAACTCA TCGCCGCCTG CCTTGCCCGC
1801 TGCTGGACAG GGGCTCGGCT GTTGGGCACT GACAATTCCG TGGTGTTGTC GGGGAAGCTG
1861 ACGTCCTTTC CATGGCTGCT CGCCTGTGTT GCCACCTGGA TTCTGCGCGG GACGTCCTTC
1921 TGCTACGTCC CTTCGGCCCT CAATCCAGCG GACCTTCCTT CCCGCGGCCT GCTGCCGGCT
1981 CTGCGGCCTC TTCCGCGTCT TCGCCTTCGC CCTCAGACGA GTCGGATCTC CCTTTGGGCC
2041 GCCTCCCCGC CTGATCAGCC TCGAAATAAA AGATCTTTAT TTTCATTAGA TCTGTGTGTT
2101 GGTTTTTTGT GTGGAGAAGC TTACAAGGAA CCCCTAGTGA TGGAGTTGGC CACTCCCTCT
2163 CTGCGCGCTC GCTCGCTCAC TGAGGCCGGG CGACCAAAGG TCGCCCGACG CCCGGGCTTT
2221 GCCCGGGCGG CCTCAGTGAG CGAGCGAGCG CGCAGAGAGG GAGTGGCCAA
SEQ ID No. 80:
scAAV-pDyn (2334 bp)
      1 TTGGCCACTC CCTCTCTGCG CGCTCGCTCG CTCACTGAGG CCGGGCGACC AAAGGTCGCC
    61 CGACGCCCGG GCTTTGCCCG GGCGGCCTCA GTGAGCGAGC GAGCGCGCAG AGAGGGAGTG
  121 GATTCGATAT CATCGATACC GTCCATATAT GGAGTTCCGC GTTACATAAC TTACGGTAAA
  181 TGGCCCGCCT GGCTGACCGC CCAACGACCC CCGCCCATTG ACGTCAATAA TGACGTATGT
  241 TCCCATAGTA ACGCCAATAG GGACTTTCCA TTGACGTCAA TGGGTGGAGT ATTTACGGTA
  301 AACTGCCCAC TTGGCAGTAC ATCAAGTGTA TCATATGCCA AGTACGCCCC CTATTGACGT
  361 CAATGACGGT AAATGGCCCG CCTGGCATTA TGCCCAGTAC ATGACCTTAT GGGACTTTCC
  421 TACTTGGCAG TACATCTACG TATTAGTCAT CGCTATTACC ATGGTCGAGG TGAGCCCCAC
  481 GTTCTGCTTC ACTCTCCCCA TCTCCCCCCC CTCCCCACCC CCAATTTTGT ATTTATTTAT
  541 TTTTTAATTA TTTTGTGCAG CGATGGGGGC GGGGGGGGGG GGGGGGCGCG CGCCAGGCGG
  601 GGCGGGGCGG GGCGAGGGGC GGGGGGGGGC GAGGCGGAGA GGTGCGGCGG CAGCCAATCA
  661 GAGCGGCGCG CTCCGAAAGT TTCCTTTTAT GGCGAGGCGG CGGCGGCGGC GGCCCTATAA
  721 AAAGCGAAGC GCGCGGCGGG CGGGAGTCGC TGCGCGCTGC CTTCGCCCCG TGCCCCGCTC
  781 CGCCGCCGCC TCGCGCCGCC CGCCCCGGCT CTGACTGACC GCGTTACTCC CACAGGTGAG
  841 CGGGCGGGAC GGCCCTTCTC CTCCGGGCTG TAATTAGCGC TTGGTTTAAT GACGGCTTGT
  901 TTCTTTTCTG TGGCTGCGTG AAAGCCTTGA GGGGCTCCGG GAGGGCCCTA GAGCCTCTGC
  961 TAACCATGTT CATGCCTTCT TCTTTTTCCT ACAGCGGCCG CGCCACCATG GCTTGGCAGG
1021 GACTTGTGCT TGCGGCTTGT CTCCTCATGT TCCCTAGTAC AACTGCTGAT TGCCTGAGCA
2081 GATGTAGCCT GTGCGCCGTG AAAACCCAGG ACGGCCCCAA GCCCATCAAC CCCCTGATCT
1141 GCAGCCTGCA GTGTCAGGCC GCTCTGCTGC CTAGCGAGGA ATGGGAGAGA TGCCAGAGCT
1201 TCCTGAGCTT CTTCACCCCC AGCACCCTGG GCCTGAACGA CAAAGAGTCT GGCAGTTTTC
2261 TGAGGAAGGA ACAGGTCAAG CGATATGGTG GCTTCCTGAG AAAGTATCCA AAGAGAAGCT
1321 CAGAAGTTGC AGGAGAAGGT GATGGGGATA GTATGGGACA CGAAGACCTG TACAAGCGCT
1381 ATGGAGGCTT TCTGAGGAGA ATACGTCCCA AACTGAAATG GGACAACCAG AAACGATATG
1441 GAGGCTTCTT GAGAAGGCAG TTCAAAGTGG TGACTAGAAG CCAAGAGGAC CCTAATGCCT
1501 ACTCAGGGGA GTTGTTTGAT GCCTGAGTCG ACCTCTGGAT TACAAAATTT GTGAAAGATT
1561 GACTGGTATT CTTAACTATG TTGCTCCTTT TACGCTATGT GGATACGCTG CTTTAATGCC
1621 TTTGTATCAT GCTATTGCTT CCCGTATGGC TTTCATTTTC TCCTCCTTGT ATAAATCCTG
1681 GTTGCTGTCT CTTTATGAGG AGTTGTGGCC CGTTGTCAGG CAACGTGGCG TGGTGTGCAC
1741 TGTGTTTGCT GACGCAACCC CCACTGGTTG GGGCATTGCC ACCACCTGTC AGCTCCTTTC
1801 CGGGACTTTC GCTTTCCCCC TCCCTATTGC CACGGCGGAA CTCATCGCCG CCTGCCTTGC
18s1 CCGCTGCTGG ACAGGGGCTC GGCTGTTGGG CACTGACAAT TCCGTGGTGT TGTCGGGGAA
2921 GCTGACGTCC TTTCCATGGC TGCTCGCCTG TGTTGCCACC TGGATTCTGC GCGGGACGTC
1981 CTTCTGCTAC GTCCCTTCGG CCCTCAATCC AGCGGACCTT CCTTCCCGCG GCCTGCTGCC
2041 GGCTCTGCGG CCTCTTCCGC GTCTTCGCCT TCGCCCTCAG ACGAGTCGGA TCTCCCTTTG
2101 GGCCGCCTCC CCGCCTGATC AGCCTCGAAA TAAAAGATCT TTATTTTCAT TAGATCTGTG
2161 TGTTGGTTTT TTGTGTGGAG AAGCTTACAA GGAACCCCTA GTGATGGAGT TGGCCACTCC
2221 CTCTCTGCGC GCTCGCTCGC TCACTGAGGC CGGGCGACCA AAGGTCGCCC GACGCCCGGG
2281 CTTTGCCCGG GCGGCCTCAG TGAGCGAGCG AGCGCGCAGA GAGGGAGTGG CCAA
SEQ ID No. 81:
scAAV-pDyn (2447 bp)
      1 TTGGCCACTC CCTCTCTGCG CGCTCGCTCG CTCACTGAGG CCGGGCGACC AAAGGTCGCC
    61 CGACGCCCGG GCTTTGCCCG GGCGGCCTCA GTGAGCGAGC GAGCGCGCAG AGAGGGAGTG
  121 GATTCGATAT CAAGCTTATC GATACCGTCG ACCTCGAGGG GGTAATAGTA ATCAATTACG
  181 GGGTCATTAG TTCATAGCCC ATATATGGAG TTCCGCGTTA CATAACTTAC GGTAAATGGC
  241 CCGCCTGGCT GACCGCCCAA CGACCCCCGC CCATTGACGT CAATAATGAC GTATGTTCCC
  301 ATAGTAACGC CAATAGGGAC TTTCCATTGA CGTCAATGGG TGGAGTATTT ACGGTAAACT
  361 GCCCACTTGG CAGTACATCA AGTGTATCAT ATGCCAAGTA CGCCCCCTAT TGACGTCAAT
  421 GACGGTAAAT GGCCCGCCTG GCATTATGCC CAGTACATGA CCTTATGGGA CTTTCCTACT
  481 TGGCAGTACA TCTACGTATT AGTCATCGCT ATTACCATGG TCGAGGTGAG CCCCACGTTC
  541 TGCTTCACTC TCCCCATCTC CCCCCCCTCC CCACCCCCAA TTTTGTATTT ATTTATTTTT
  601 TAATTATTTT GTGCAGCGAT GGGGGCGGGG GGGGGGGGGG GGCGCGCGCC AGGCGGGGCG
  661 GGGGGGGGCG AGGGGCGGGG CGGGGCGAGG CGGAGAGGTG CGGCGGCAGC CAATCAGAGC
  721 GGCGCGCTCC GAAAGTTTCC TTTTATGGCG AGGGGGGGGC GGGGGGGGCC CTATAAAAAG
  781 CGAAGCGCGC GGCGGGGGGG AGTCGCTGCG CGCTGCCTTC GCCCCGTGCC CCGCTCCGCC
  841 GCCGCCTCGC GCCGCCCGCC CCGGCTCTGA CTGACCGCGT TACTCCCACA GGTGAGCGGG
  901 CGGGACGGCC CTTCTCCTCC GGGCTGTAAT TAGCGCTTGG TTTAATGACG GCTTGTTTCT
  061 TTTCTGTGGC TGCGTGAAAG CCTTGAGGGG CTCCGGGAGG GCCCTAGAGC CTCTGCTAAC
1021 CATGTTCATG CCTTCTTCTT TTTCCTACAG CGGCCGCGCC ACCATGCCAA GATCATGCTG
1081 TTCACGTAGT GGAGCCCTTC TCTTGGCACT TCTTCTGCAA GCAAGCATGG AAGTCAGAGG
1141 TTGGTGTTTG GAGAGTTCCC AATGCCAAGA CCTGACTACT GAGTCAAACC TGCTGGAATG
1201 CATAAGAGCC TGTAAACCCA AGGAACAGGT GAAACGCTAT GGTGGGTTTC TCAGGAAATA
1261 TCCCAAAAGA TCCTCTGAGG TTGCTGGTGA AGGTGATGGG GACAGTATGG GCCATGAGGA
1321 CCTGTACAAG AGATATGGTG GATTCCTCAG AAGAATCAGA CCCAAACTGA AGTGGGACAA
1381 CCAGAAGAGA TATGGAGGGT TTCTGAGGAG ACAGTTCAAG GTAGTCACCA GATCCCAAGA
1441 GGACCCTAAC GCGTATAGTG GGGAACTCTT TGATGCATGA GTCGACCTCT GGATTACAAA
1501 ATTTGTGAAA GATTGACTGG TATTCTTAAC TATGTTGCTC CTTTTACGCT ATGTGGATAC
1561 GCTGCTTTAA TGCCTTTGTA TCATGCTATT GCTTCCCGTA TGGCTTTCAT TTTCTCCTCC
1621 TTGTATAAAT CCTGGTTGCT GTCTCTTTAT GAGGAGTTGT GGCCCGTTGT CAGGCAACGT
1681 GGCGTGGTGT GCACTGTGTT TGCTGACGCA ACCCCCACTG GTTGGGGCAT TGCCACCACC
1741 TGTCAGCTCC TTTCCGGGAC TTTCGCTTTC CCCCTCCCTA TTGCCACGGC GGAACTCATC
1801 GCCGCCTGCC TTGCCCGCTG CTGGACAGGG GCTCGGCTGT TGGGCACTGA CAATTCCGTG
1861 GTGTTGTCGG GGAAGCTGAC GTCCTTTCCA TGGCTGCTCG CCTGTGTTGC CACCTGGATT
1921 CTGCGCGGGA CGTCCTTCTG CTACGTCCCT TCGGCCCTCA ATCCAGCGGA CCTTCCTTCC
1981 CGCGGCCTGC TGCCGGCTCT GCGGCCTCTT CCGCGTCTTC GCCTTCGCCC TCAGACGAGT
2041 CGGATCTCCC TTTGGGCCGC CTCCCCGCCT GATCAGCCTC GACTGTGCCT TCTAGTTGCC
2101 AGCCATCTGT TGTTTGCCCC TCCCCCGTGC CTTCCTTGAC CCTGGAAGGT GCCACTCCCA
2161 CTGTCCTTTC CTAATAAAAT GAGGAAATTG CATCGCATTG TCTGAGTAGG TGTCATTCTA
2221 TTCTGGGGGG TGGGGTGGGG CAGGACAGCA AGGGGGAGGA TTGGGAAGAC AATAGCAGGC
2281 ATGCTGGGGA GAGAAGCTTA CAAGGAACCC CTAGTGATGG AGTTGGCCAC TCCCTCTCTG
2341 CGCGCTCGCT CGCTCACTGA GGCCGGGCGA CCAAAGGTCG CCCGACGCCC GGGCTTTGCC
2401 CGGGCGGCCT CAGTGAGCGA GCGAGCGCGC AGAGAGGGAG TCGCCAA
```

Particular embodiments of the present invention are
1. A delivery vector comprising a DNA sequence encoding pre-prodynorphin or pre-prodynorphin-variants and
   wherein said delivery vector drives expression of a pre-propeptide in a target cell, and
   wherein said delivery vector comprising said DNA sequence enables the release of dynorphin or dynorphin-variants from the target cell on demand, and
   wherein said pre-propeptide is pre-prodynorphin or a pre-prodynorphin-variant and
   wherein said pre-propeptide comprises a signal peptide, wherein the signal peptide is a N-terminal extension of a nascent polypeptide chain and wherein said signal peptide mediates protein targeting to the lumen of the endoplasmatic reticulum, and,
   wherein said pre-propeptide comprises either (i) a N-terminal pro-peptide fragment on the C-terminal end of said signal peptide and wherein said N-terminal pro-peptide fragment comprises a sorting motif comprising the elements DL and EX_{y}L, in particular a sorting motif consisting of the amino acid sequence DLXₓEX_{y}L (SEQ ID No. 36), where x is an integer from 1 to 20, y is an integer from 1 to 10, and each instance of X may independently be any amino acid (for the avoidance of doubt, this means that in the present invention in the first sequence of e.g. 1 to 20 X, each X may individually be any amino acid, and in the second sequence of e.g. 1 to 10 X, each X may individually be any amino acid, particularly as further defined herein), or wherein said pre-propeptide comprises (ii) a N-terminal pro-peptide fragment of a pre-pro-neuropeptide or protein sorted to large dense core vesicles, other than pre-prodynorphin, on the C-terminal end of said signal peptide and wherein said N-terminal pro-peptide fragment comprises a sorting motif of said pre-pro-neuropeptide or protein sorted to large dense core vesicles, and wherein said N-terminal pro-peptide fragment consists of 16 to 90 amino acids, and
   wherein said pre-prodynorphin or pre-prodynorphin-variants comprise at least one of the following sequences selected from the group:
   a. Dyn A that is SEQ ID No. or a variant thereof consisting of the first 13 amino acids from the N-terminal end or a variant thereof consisting of the first 8 amino acids from the N-terminal end
   b. Dyn B that is SEQ ID No. 3
   c. leumorphin that is SEQ ID No. 4
   d. variants of Dyn A, said variants having an amino acid sequence identity of at least 60% within the first 8 amino acids from the N-terminal end of SEQ ID No. 2,
   e. variants of Dyn B, said variants having an amino acid sequence identity of at least 60% within the first 8 amino acids from the N-terminal end of SEQ ID No. 3,
   f. variants of leumorphin, said variants having an amino acid sequence identity of at least 60% within the first 8 amino acids from the N-terminal end of SEQ ID No. 4.
2. A delivery vector comprising a DNA sequence encoding pre-prodynorphin or pre-prodynorphin-variants according to embodiment 1, wherein said N-terminal pro-peptide fragment consists of 20 to 90 amino acids, preferably 30 and 90 amino acids.
3. A delivery vector comprising a DNA sequence encoding pre-prodynorphin or pre-prodynorphin-variants according to embodiment 1 or 2, wherein said N-terminal pro-peptide fragment on the C-terminal end of the signal peptide is a modified propeptide fragment of ppDyn wherein the unmodified propeptide fragment of ppDyn is

SEQ ID No. 5
DCLSRCSLCA VKTQDGPKPI NPLICSLQCQ AALLPSEEWE
RCQSFLSFFT PSTLGLNDKE DLGSKSVGEG PYSELAKLSG
SFLKELEKSK FLPSISTKEN TLSKSLEEKL RGLSDGFREG
AESELMRDAQ LNDGAMETGT LYLAEEDPKE QV

and wherein the modification of said propeptide fragment of ppDyn is a shortening;
or the modification is a replacement of parts of SEQ ID No. 5 with a propeptide of or a fragment of a propeptide of a neuropeptide;
wherein the modified propeptide fragment of ppDyn i) comprises at least one sorting motif comprising the elements DL and EX_{y}L, in particular a sorting motif consisting of the amino acid sequence DLXxEXyL (SEQ ID No. 36) or ii) comprises a sorting motif of said pre-pro-neuropeptide or protein sorted to large dense core vesicles other than pre-prodynorphin.
4. A delivery vector comprising a DNA sequence encoding pre-prodynorphin or pre-prodynorphin-variants according to any of embodiments 1 to 3, wherein said N-terminal pro-peptide fragment on the C-terminal end of the signal peptide is a modified propeptide fragment of ppDyn that comprises or consists of

SEQ ID No. 6
DCLSRCSLCA VKTQDGPKPI NPLICSLQCQ AALLPSEEWE
RCQSFLSFFT PSTLGLNDKE DLGSKSVGEG PYSELAKLSG
SFLRKEQVKR

5. A delivery vector comprising a DNA sequence encoding pre-prodynorphin or pre-prodynorphin-variants according to any of embodiments 1 to 3, wherein said N-terminal pro-peptide fragment on the C-terminal end of the signal peptide is a modified propeptide fragment of ppDyn that comprises or consists of

SEQ ID No. 7
DLGSKSVGEG PYSELAKLSG SFLKELEKSK FLPSISTKEN
TLSKSLEEKL RGLSDGFREG AESELMRDAQ LNDGAMETGT
LYLAEEDPKE QV
or
SEQ ID No. 8
DLGSKSVGEG PYSELAKLSG SFLRKE QV
or
SEQ ID No 9
DLGSKSVGEG PYSELAKLRKE QV.

6. A delivery vector comprising a DNA sequence encoding pre-prodynorphin or pre-prodynorphin-variants according to embodiment 3, wherein the modification is a replacement of parts of SEQ ID No. 5 with a propeptide of or a fragment of a propeptide of a neuropeptide; wherein the modified propeptide fragment of ppDyn i) comprises at least one sorting motif comprising the elements DL and EX_{y}L, in particular a sorting motif consisting of the amino acid sequence DLXₓEX_{y}L (SEQ ID No. 36) or ii) comprises a sorting motif of said pre-pro-neuropeptide or protein sorted to large dense core vesicles other than pre-prodynorphin.
7. A delivery vector comprising a DNA sequence encoding pre-prodynorphin or pre-prodynorphin-variants according to embodiment 6 wherein the modified propeptide fragment comprises or consists of

SEQ ID No. 10:
MPRSCCSRSG ALLLALLLQA SMEVRGWCLE SSQCQDLTTE
SNLLECIRAC KP

8. A delivery vector comprising a DNA sequence encoding pre-prodynorphin or pre-prodynorphin-variants according to embodiment 6 wherein the modified propeptide fragment comprises or consists of a propeptide fragment of preproEnkephalin, preproBDNF, preproTachykinin, prepro-Somatostatin, pre-pro-VIP, prepro-CCK, preproNociceptin or preproNPY, wherein said propeptide fragment comprises a sorting motif, in particular said propeptide fragment maybe selected from the group comprising of any of SEQ ID Nos: 37 to 52.
9. A delivery vector comprising a DNA sequence encoding pre-prodynorphin or pre-prodynorphin-variants according to embodiments 1-8 wherein the modified propeptide fragment is optionally flanked by peptidase recognition signals comprising K, R, KR, RK or RR.
10. A delivery vector comprising a DNA sequence encoding pre-prodynorphin or pre-prodynorphin-variants according to any of embodiments 1-9, wherein the target cell are neuronal cells of the central nervous system.
11. A delivery vector comprising a DNA sequence encoding pre-prodynorphin or pre-prodynorphin-variants according to any of embodiments 1-10, wherein the signal peptide is a peptide sequence of from 10 to 30 amino acids at the N-terminal end of precursor-proteins that are destined into the lumen of the endoplasmatic reticulum.
12. A delivery vector comprising a DNA sequence encoding pre-prodynorphin or pre-prodynorphin-variants according to any of embodiments 1-11, wherein the signal peptide is selected from the group comprising:

(SEQ ID No. 11)
MAWQGLVLAA CLLMFPSTTA
(SEQ ID No. 12)
MARFLTLCTW LLLLGPGLLA TVRA
(SEQ ID No. 13)
MLGNKRLGLS GLTLALSLLV CLGALAEA
(SEQ ID No. 14)
MLSCRLQCAL AALSIVLALG CVTG
(SEQ ID No. 15)
MKILVALAVF FLVSTQLFA
(SEQ ID No. 16)
MRIMLLFTAI LAFSLA
(SEQ ID No. 17)
MPRSCCSRSG ALLLALLLQA SMEVRG
(SEQ ID No. 18)
MNSGVCLCVL MAVLAAGA
(SEQ ID No. 19)
MKVLLCDLLL LSLFSSVFS
(SEQ ID NO. 20)
MQPTLLLSLL GAVGLAAVNS

13. A delivery vector according to any of embodiments 1-12, wherein said delivery vector leads to release-on-demand of dynorphins or dynorphin-variants with agonistic effects on human Kappa Opioid Receptors.
14. A delivery vector according to any of embodiments 1-13, wherein the dynorphin variants have an amino acid sequence identity of at least 70% within the first 8 amino acids from the N-terminal end of SEQ ID No. 2 (YGGFLRRI), SEQ ID No. 3 (YGGFLRRQ) or SEQ ID No. 4 (YGGFLRRQ), respectively.
15. A delivery vector according to any of embodiments 1-14, wherein the variants have an amino acid sequence identity of at least 80% within the first 8 amino acids from the N-terminal end of SEQ ID No. 2, SEQ ID No. 3 or SEQ ID No. 4, respectively.
16. A delivery vector according to any of embodiments 1-15, wherein said delivery vector comprises in addition a recombinant adeno-associated virus (AAV) vector genome or a recombinant lentivirus genome.
17. A delivery vector according to any of embodiments 1-16 comprising a recombinant adeno-associated virus (AAV) vector genome comprising inverted terminal repeats (ITR) preferably derived from AAV serotype 2, alternatively from AAV serotype 1, 3, 4, 5, 6, 7, 8, 9, 10, rh10, 11, 12, 13, 14, or reengineered variants thereof, wherein said vector genome is packaged in an AAV capsid selected from the group comprising AAV serotypes 1, 2, 3, 4, 5, 6, 7, 8, 9, 10, rh10, 11, 12, 13, 14, serpentine AAV, ancestral AAV, AAV-TT, AAVv66, AAV1P4, AAV1P5, AAV-PHP.B, AAV-PHP.eB, AAV2-HBKO, AAV.CAP-B10, AAV.CAP-MAC, AAV2.NN, or further AAV capsid mutants derived thereof or a chimeric vector comprising capsid proteins derived from two or more, preferably two of the aforementioned AAV serotype capsids; in particular embodiments, the capsids are capsids derived from AAV serotype 1 and/or 2.
   In certain embodiments, the delivery vector according to embodiment 1-16 comprises a recombinant adeno-associated virus (AAV) vector genome comprising inverted terminal repeats (ITR) preferably derived from AAV serotype 2, alternatively from AAV serotype 1, 3, 4, 5, 6, 7, 8, 9, 10, rh10, 11, 12, 13, 14, or reengineered variants thereof, wherein said vector genome is packaged in an AAV capsid selected from the group comprising AAV serotypes 1, 2, 3, 4, 5, 6, 7, 8, 9, 10, rh10, 11, 12, 13, 14, serpentine AAV, ancestral AAV, AAV-TT, AAVv66, AAV1P4, AAV1P5, AAV-PHP.B, AAV-PHP.eB, AAV2-HBKO, AAV.CAP-B10, AAV.CAP-MAC, AAV2.NN, or further AAV capsid mutants derived thereof, preferably of AAV serotype 1 or 2.
18. A delivery vector comprising a DNA sequence encoding pre-prodynorphin or pre-prodynorphin-variants according to any of the preceding embodiments.
19. A recombinant virus particle or a liposome or nanoparticle, comprising a delivery vector according to any of the preceding embodiments.
20. The recombinant virus particle or liposome or nanoparticle of embodiment 19, wherein said delivery vector comprises in addition a recombinant adeno-associated virus (AAV) vector genome and said rAAV vector genome is encapsidated in an AAV capsid or wherein said delivery vector comprises in addition a recombinant lentivirus vector genome and is packaged in a lentivirus particle.
21. A delivery vector or recombinant virus particle or liposome or nanoparticle according to any of embodiments 1-20 for use in delivering a nucleic acid to a cell of the central nervous system, comprising contacting the cell with the delivery vector or recombinant virus particle or liposome or nanoparticle under conditions sufficient for the DNA sequence encoding pre-prodynorphin or pre-prodynorphin-variants to be introduced into the cell.
22. A delivery vector or recombinant virus particle or liposome or nanoparticle according to any of embodiments 1-21 for use as medicament.
23. A delivery vector or recombinant virus particle or liposome or nanoparticle according to any of embodiments 1 to 21 for use in treating focal epilepsy in a subject, in particular mesial temporal lobe epilepsy, or for use in preventing epileptic seizures in a subject that suffers from focal epilepsy whereby said delivery vector or recombinant virus particle or liposome or nanoparticle provides activation of human Kappa Opioid Receptors in the epileptogenic focus, thereby inhibiting seizures.

It is apparent to the skilled person that, as detailed herein, the delivery vector or recombinant virus particle or liposome or nanoparticle provides activation of human Kappa Opioid Receptors in the epileptogenic focus, thereby inhibiting seizures, through inducing production of corresponding peptides, as further detailed herein.
24. DNA sequence selected from the group comprising the following sequences: SEQ ID No. 69, SEQ ID No. 70, SEQ ID No. 71, SEQ ID No. 72, and SEQ ID No. 73.
25. A delivery vector comprising a DNA sequence according to embodiment 24.
26. A delivery vector according to embodiment 25, wherein said delivery vector comprises in addition a recombinant adeno-associated virus (AAV) vector genome or a recombinant lentivirus genome.
27. A delivery vector according to embodiment 25 or 26 comprising a recombinant adeno-associated virus (AAV) vector genome comprising inverted terminal repeats (ITR), preferably derived from AAV serotype 2, alternatively from AAV serotype 1, 3, 4, 5, 6, 7, 8, 9, 10, rh10, 11, 12, 13, 14, or reengineered variants thereof, wherein said vector genome is packaged in an AAV capsid selected from the group comprising AAV serotypes 1, 2, 3, 4, 5, 6, 7, 8, 9, 10, rh10, 11, 12, 13, 14, serpentine AAV, ancestral AAV, AAV-TT, AAVv66, AAV1P4, AAV1P5, AAV-PHP.B, AAV-PHP.eB, AAV2-HBKO, AAV.CAP-B10, AAV.CAP-MAC, AAV2.NN, or further AAV capsid mutants derived thereof or a chimeric vector or a chimeric vector comprising capsid proteins derived from two or more, preferably two of the aforementioned AAV serotype capsids; in particular embodiments, the capsids are capsids derived from AAV serotype 1 and/or 2.
   In certain embodiments, the delivery vector according to embodiment 25 or 26 comprises a recombinant adeno-associated virus (AAV) vector genome comprising inverted terminal repeats (ITR), preferably derived from AAV serotype 2, alternatively from AAV serotype 1, 3, 4, 5, 6, 7, 8, 9, 10, rh10, 11, 12, 13, 14, or reengineered variants thereof, wherein said vector genome is packaged in an AAV capsid selected from the group comprising AAV serotypes 1, 2, 3, 4, 5, 6, 7, 8, 9, 10, rh10, 11, 12, 13, 14, serpentine AAV, ancestral AAV, AAV-TT, AAVv66, AAV1P4, AAV1P5, AAV-PHP.B, AAV-PHP.eB, AAV2-HBKO, AAV.CAP-B10, AAV.CAP-MAC, AAV2.NN, or further AAV capsid mutants derived thereof, preferably two of the aforementioned AAV capsids, preferably of AAV serotype 1 and 2.
28. A delivery vector according to any of embodiments 25-27, wherein said delivery vector comprises in addition at least one sequence selected from the group comprising SEQ ID No. 59, SEQ ID No. 60, SEQ ID No. 61, SEQ ID No. 63, SEQ ID No. 64, SEQ ID No. 65, SEQ ID No. 66, SEQ ID No. 67, and SEQ ID No. 68.
29. A delivery vector according to any of embodiments 25-28, wherein said delivery vector comprises a sequence selected from the group comprising SEQ ID No. 74, SEQ ID No. 75, SEQ ID No. 76, SEQ ID No. 77, SEQ ID No. 78, SEQ ID No. 79, SEQ ID No. 80, and SEQ ID No. 81.
30. A recombinant virus particle or a liposome or nanoparticle, comprising a delivery vector according to any of embodiments 25-29.
31. The recombinant virus particle or liposome or nanoparticle according to embodiment 30, wherein said delivery vector comprises in addition a recombinant adeno-associated virus (AAV) vector genome and said rAAV vector genome is encapsidated in an AAV capsid or wherein said delivery vector comprises in addition a recombinant lentivirus vector genome and is packaged in a lentivirus particle.
32. A delivery vector or recombinant virus particle or liposome or nanoparticle according to any of embodiments 25-31 for use in delivering a nucleic acid to a cell of the central nervous system, comprising contacting the cell with the delivery vector or recombinant virus particle or liposome or nanoparticle under conditions sufficient for a DNA comprising a sequence selected from the group comprising the SEQ ID No. 69, SEQ ID No. 70, SEQ ID No. 71, SEQ ID No. 72, and SEQ ID No. 73 to be introduced into the cell.

In particular embodiments, the DNA comprising a sequence selected from the group comprising the SEQ ID Nos. 69-73 to be introduced into the cell is a DNA spected from SEQ ID Nos. 74-81 (AAV genomes), and, in the case of lentiviruses, a DNA wherein a sequence selected from the group comprising the SEQ ID Nos. 69-73 and regulatory sequences are embedded in a lentivirus genome.
33. A delivery vector or recombinant virus particle or liposome or nanoparticle according to any of embodiments 25-32 for use as medicament.
34. A delivery vector or recombinant virus particle or liposome or nanoparticle according to any of embodiments 25-33 for use in treating epilepsy in a subject, in particular focal epilepsy, in particular mesial temporal lobe epilepsy, or for use in preventing epileptic seizures in a subject that suffers from focal epilepsy, wherein said delivery vector or recombinant virus particle or liposome or nanoparticle provides activation of human Kappa Opioid Receptors in the epileptogenic focus, thereby inhibiting seizures.

### FIGURE DESCRIPTION

FIG. 1: Overview of the shortening of the human ppDyn cDNA.

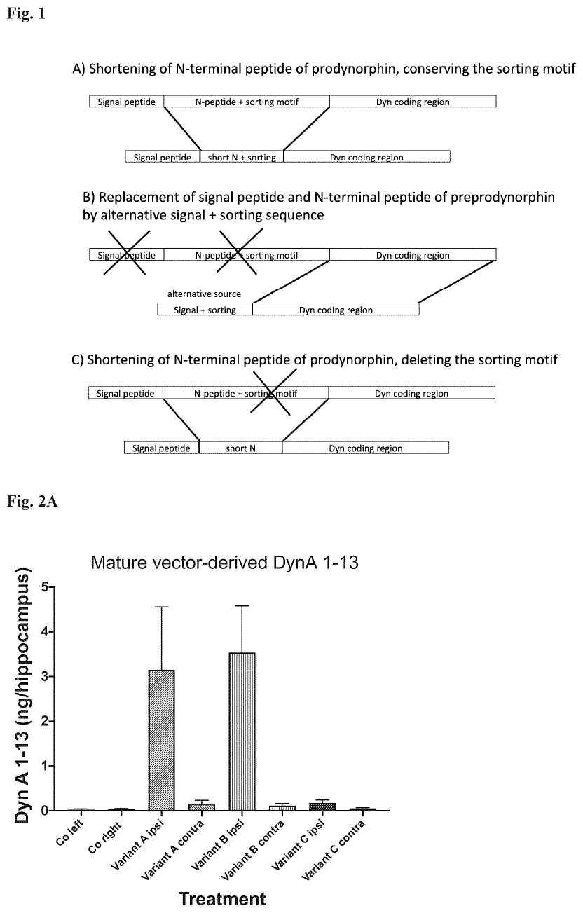

FIG. 2: Results of an ELISA to measure the content of (A) dynorphin A (DynA) and (B) dynorphin B (DynB) after intraparenchymal CNS transduction of AAV vectors expressing the indicated ppDyn variants. Variant A=shortened N-peptide preserving the sorting motif. Variant B=signal and N-Peptide replaced by POMC signal and sorting motif. Variant C=shortened N-peptide with deleted sorting motif. ipsi=site of AAV transduction, contra=non-transduced (control) site.

FIG. 3: Seizure suppression by two scAAV vector variants containing shortened pDyn cDNA or with alternative signal and sorting sequence. Data represent N±SEM (N=3).

FIG. 4: Overview of DNA-sequence elements of AAV-pDyn vector variants.

Displayed are the DNA sequence elements of the displayed AAV vectors. SEQ ID numbering refers to the numbering in the body of the text.

Dark grey box: DNA sequence elements derived from AAV serotype 2. ITR=inverted terminal repeat, ΔITR=inverted terminal repeat with deleted terminal resolution site as used in self-complementary (sc)AAV vectors.

Light grey box: DNA sequence elements for promoters or other regulatory elements of gene expression. CBA prom.=cytomegalovirus (CMV) enhancer fused to the chicken beta-actin promoter followed by a chimeric intron. sCBA prom.=shortened cytomegalovirus (CMV) enhancer fused to chicken beta-actin promoter followed by a chimeric intron. hSyn prom.=human synapsin promoter. WPRE=woodchuck hepatitis virus posttranscriptional regulatory element, bGH pA+=polyA+ signal sequence of the bovine growth hormone gene. SpA+=synthetic poly A+ signal sequence.

White box: DNA sequence elements for the human ppDyn cDNA. ppDyn cDNA sequences are codon-optimized. Codon optimized version 1 is used for SEQ ID No. 74 and SEQ ID No. 75. Codon optimized version 2 is used for SEQ ID No. 76 to 80, and for the pDyn part of SEQ ID No. 81. Pre=DNA sequence for the signal sequence of ppDyn, pro=DNA sequence covering the N-peptide of ppDYN, pro1, pro2, and proDs=different shortened versions of “pro” referring to the N-peptide of ppDyn. POMC=cDNA sequence of the N-terminal part of neuropeptide POMC spanning the pre and pro elements and replacing those of ppDyn. pDyn=prodynorphin.

FIG. 5: Reduction of seizure activity after injection of AAV-pDyn expressing Seq ID No. 70. Hpds, generalized seizures and spike trains were measured over periods of 48 hours each time-interval. Data are shown as % of pretreatment seizure activity for Hpds and spike trains (left y-axis). Generalized seizures are given as absolute number of seizures (right y-axis. N=7-9, p=0.0025 for Hpds, <0.0001 for spike trains and 0.0001 for generalized seizures (one-way ANOVA). Mice show a significant reduction of drug-resistant focal seizures starting from 7 days after treatment. Generalized seizures are almost completely abolished after 4 weeks.

FIG. 6: Reduction of seizure activity after injection of AAV-pDyn expressing Seq ID No. 77. HPDs were measured over periods of 48 hours each time-interval. Data are shown as time spent in HPDs. N =2-3. Mice show a marked reduction of drug-resistant focal seizures starting from 10 days after AAV delivery.

### REFERENCES

Ausubel, F. M. et al. (eds.), Current Protocols in Molecular Biology, Greene Publishing Associates, Wiley (1989)
Berns K. I. and C. R. Parrish in FIELDS VIROLOGY, eds. D. N. Knipe and P. M. Howley, volume 2, chapter 57 (6th ed., 2013, Wolters-Kluwer, Lippincott Williams & Wilkins Publishers).
Borner, K., E. Kienle, L. Y. Huang, J. Weinmann, A. Sacher, P. Bayer, C. Stullein, J. Fakhiri, L. Zimmermann, A. Westhaus, J. Beneke, N. Beil, E. Wiedtke, C. Schmelas, D. Miltner, A. Rau, H. Erfle, H. G. Krausslich, M. Muller, M. Agbandje-McKenna and D. Grimm (2020). “Pre-arrayed Pan-AAV Peptide Display Libraries for Rapid Single-Round Screening.” Mol Ther 28(4): 1016-1032
Challis, R. C., S. Ravindra Kumar, X. Chen, D. Goertsen, G. M. Coughlin, A. M. Hori, M. R. Chuapoco, T. S. Otis, T. F. Miles and V. Gradinaru (2022). “Adeno-Associated Virus Toolkit to Target Diverse Brain Cells.” Annu Rev Neurosci 45: 447-469.
Choi, J. H., N. K. Yu, G. C. Baek, J. Bakes, D. Seo, H. J. Nam, S. H. Baek, C. S. Lim, Y. S. Le and B. K. Kaang (2014). “Optimization of AAV expression cassettes to improve packaging capacity and transgene expression in neurons.” Mol Brain 7: 17.
Coatsworth J J. Studies on the clinical effect of marketed antiepileptic drugs. 1971. NINDS Monograph 12
de Lanerolle N C, Williamson A, Meredith C, Kim J H, Tabuteau H, Spencer D D, Brines M L. Dynorphin and the kappa 1 ligand [3h]u69,593 binding in the human epileptogenic hippocampus. 1997. Epilepsy Res 28:189-205.
During et al. (2003) Methods Mol Med 76: 221-36
Elmore, Z. C., L. Patrick Havlik, D. K. Oh, L. Anderson, G. Daaboul, G. W. Devlin, H. A. Vincent and A. Asokan (2021). “The membrane associated accessory protein is an adeno-associated viral egress factor.” Nat Commun 12(1): 6239.
Engel J J. Mesial temporal lobe epilepsy: What have we learned? 2001. Neuroscientist 7:340-352.
Gambardella A, Manna I, Labate A, Chifari R, Serra P, La Russa A, LePiane E, Cittadella R, Andreoli V, Sasanelli F, Zappia M, Aguglia U, Quattrone A. Prodynorphin gene promoter polymorphism and temporal lobe epilepsy. 2003. Epilepsia 44:1255-1256.
Gossen M and Bujard H, (1992) Tight control of gene expression in mammalian cells by tetracyclin-responsive promoters. Proc. Natl. Acad. Sci. USA 89:5547-51.
Gossen M, Freundlieb S, Bender G, Müller G, Hillen W and Bujard H, (1995) Transcriptional activation by tetracyclines in mammalian cells. Science 268(5218):1766-9.
Grimm D, Kay M A, Kleinschmidt J A (2003) Helper virus-free, optically controllable, and two-plasmid-based production of adeno-associated virus vectors of serotypes 1 to 6, Mol Ther 7(6) 839-50
Harvey D M, Caskey C T (1998) Inducible control of gene expression: prospects for gene therapy. Curr. Opin. Chem. Biol. 2:512-8.
Hauck B, Chen L, and Xiao W, Generation and Characterization of chimeric recombinant AAV vectors. (2003) Molecular Therapy 7, No. 3, 419-425.
Henriksen S J, Chouvet G, McGinty J, Bloom F E. Opioid peptides in the hippocampus: Anatomical and physiological considerations. 1982. Ann N Y Acad Sci 398:207-220.
Hsu, H. L., A. Brown, A. B. Loveland, A. Lotun, M. Xu, L. Luo, G. Xu, J. Li, L. Ren, Q. Su, D. J. Gessler, Y. Wei, P. W. L. Tai, A. A. Korostelev and G. Gao (2020). “Structural characterization of a novel human adeno-associated virus capsid with neurotropic properties.” Nat Commun 11(1): 3279.
Hiser D, Gogol-Döring A, Chen W, Heilbronn R (2014) Adeno-associated virus type 2 wild-type and vector-mediated genomic integration profiles in human diploid fibroblasts analyzed by 3^{rd} generation PacBio DNA sequencing. J Virol 188 (19): 11253-11263
Kimura et al (2023) A mosaic adeno-associated virus vector as a versatile tool that exhibits high levels of transgene expression and neuron speci!city in primate brain. Nature Communications 14:4762.
Li, C. and R. J. Samulski (2020). “Engineering adeno-associated virus vectors for gene therapy.” Nat Rev Genet 21(4): 255-272.
Loacker S, Sayyah M, Wittmann W, Herzog H, Schwarzer C. Endogenous dynorphin in epileptogenesis and epilepsy: Anticonvulsant net effect via kappa opioid receptors. 2007. Brain 130:1017-1028.
Loeb, J. E., W. S. Cordier, M. E. Harris, M. D. Weitzman and T. J. Hope (1999). “Enhanced expression of transgenes from adeno-associated virus vectors with the woodchuck hepatitis virus posttranscriptional regulatory element: implications for gene therapy.” Hum Gene Ther 10(14): 2295-2305.
Loftus S K, Erickson R P, Walkley S U, Bryant M A, Incao A, Heidenreich R A, Pavan W J (2002), Rescue of neurodegeneration in Niemann-Pick C mice by a prion promoter-driven Npc1 cDNA transgene. Hum. Mol. Genet. 11:3107-14.
Loscher W, Schmidt D. Modern antiepileptic drug development has failed to deliver: Ways out of the current dilemma. 2011. Epilepsia 52:657-678.
Magari S R Rivera V M, Iulicci J D, Gilman M, Cerasoli F Jr, (1997) Pharmacologic control of a humanized gene therapy system implanted into nude mice J. Clin. Invest. 100:2865-72
Mattola, S., V. Aho, L. F. Bustamante-Jaramillo, E. Pizzioli, M. Kann and M. Vihinen-Ranta (2022). “Nuclear entry and egress of parvoviruses.” Mol Microbiol. 00: 1-14
McCarty D M, Monahan P E, Samulski R J (2001) Self-complementary recombinant adeno-associated virus (scAAV) vectors promote efficient transduction independently of DNA synthesis, Gene Ther 8(16) 1248-54
McNamara J O. Emerging insights into the genesis of epilepsy. 1999. Nature 399:A15-22.
Mietzsch M, Broecker F, Reinhardt A, Seeberger P H, Heilbronn R (2014) Differential adeno-associated virus serotype-specific interaction patterns with synthetic heparins and other glycans. J Virol 88: 2991-3003
Mietzsch M, Grasse S, Zurawski C, Weger S, Bennett A, Agbandje-McKenna M, Muzyczka N, Zolotukhin S, Heilbronn R (2014) OneBac: Platform for scalable and high-titer production of adeno-associated virus serotype 1-12 vectors for gene therapy. Hum Gene Ther 25: 212-222
Muzyczka (1992) Use of adeno-associated virus as a general transduction vector for mammalian cells. Curr. Topics Microbiol. Immunol. 158:97-129).
Naidoo, J., L. M. Stanek, K. Ohno, S. Trewman, L. Samaranch, P. Hadaczek, C. O'Riordan, J. Sullivan, W. San Sebastian, J. R. Bringas, C. Snieckus, A. Mahmoodi, A. Mahmoodi, J. Forsayeth, K. S. Bankiewicz and L. S. Shihabuddin (2018). “Extensive Transduction and Enhanced Spread of a Modified AAV2 Capsid in the Non-human Primate CNS.” Mol Ther 26(10): 2418-2430.
Nicolson, S. C. and R. J. Samulski (2014). “Adeno-associated virus utilizes host cell nuclear import machinery to enter the nucleus.” J Virol. 88(8): 4132
No D, Yao TP Evans RM (1996) Ecdysone-inducible gene expression in mammalian cells and transgenic mice Proc. Natl. Acad. Sci. USA 93:3346.-51
Noé et al, (2008) Neuropeptide Y gene therapy decreases chronic spontaneous seizures in a rat model of temporal lobe epilepsy. Brain 131, 1506-1515.
Pavlou, M., C. Schon, L. M. Occelli, A. Rossi, N. Meumann, R. F. Boyd, J. T. Bartoe, J. Siedlecki, M. J. Gerhardt, S. Babutzka, J. Bogedein, J. E. Wagner, S. G. Priglinger, M. Biel, S. M. Petersen-Jones, H. Buning and S. Michalakis (2021). “Novel AAV capsids for intravitreal gene therapy of photoreceptor disorders.” EMBO Mol Med 13(4): e13392.
Pillay S, Meyer N L, Puschnik A S, Davulcu O, Diep J, Ishikawa Y, Jae L T, Wosen J E, Nagamine C M, Chapman M S, Carette J E (2016) Nature 530 (7588) 108-12.
Pirker S, Gasser E, Czech T, Baumgartner C, Schuh E, Feucht M, Novak K, Zimprich F, Sperk G. Dynamic up-regulation of prodynorphin transcription in temporal lobe epilepsy. 2009. Hippocampus 19:1051-1054.
Rosenzweig A (2007), Vectors for Gene Therapy. In: Current Protocols in Human Genetics. Wiley John and Sons, Inc.: DOI: 10.1002/0471142905.hg1200s52.
Schunk E, Aigner C, Stefanova N, Wenning G, Herzog H, Schwarzer C. Kappa opioid receptor activation blocks progressive neurodegeneration after kainic acid injection. 2011. Hippocampus 21:1010-1020.
Schwarzer C. 30 years of dynorphins—new insights on their functions in neuropsychiatric diseases. 2009. Pharmacol Ther 123:353-370.
Siggins G R, Henriksen S J, Chavkin C, Gruol D. Opioid peptides and epileptogenesis in the limbic system: Cellular mechanisms. 1986. Adv Neurol 44:501-512.
Simonato M, Romualdi P. Dynorphin and epilepsy. 1996. Prog Neurobiol 50:557-583.
Solbrig M V, Adrian R, Chang D Y, Perng G C. Viral risk factor for seizures: Pathobiology of dynorphin in herpes simplex viral (hsv-1) seizures in an animal model. 2006. Neurobiol Dis 23:612-620.
Sonntag, F., K. Schmidt and J. A. Kleinschmidt (2010). “A viral assembly factor promotes AAV2 capsid formation in the nucleolus.” Proc Natl Acad Sci USA 107(22): 10220-10225.
Spencer S, Huh L. Outcomes of epilepsy surgery in adults and children. 2008. Lancet Neurol 7:525-537.
Stogmann E, Zimprich A, Baumgartner C, Aull-Watschinger S, Hollt V, Zimprich F. A functional polymorphism in the prodynorphin gene promotor is associated with temporal lobe epilepsy. 2002. Ann Neurol 51:260-263.
Stutika C, Gogol-Doring A, Botscben L, Mietzsch M, Weger S, Feldkamp M, Chen W, Heilbronn R (2015) A comprehensive RNA-Seq analysis of the adeno-associated virus type 2 transcriptome reveals novel AAV transcripts, splice variants, and derived proteins. J Virol 90(3) 1278-89
Suriano et al. (2021) bioRxiv preprint doi: https://doi.org/10.1101/2021.09.28.462148
Takahashi M, Senda T, Tokuyama S, Kaneto H. Further evidence for the implication of a kappa-opioid receptor mechanism in the production of psychological stress-induced analgesia. 1990. Jpn J Pharmacol 53:487-494.
Tani M, Fuentes M E, Petersen J W, Trapp B D, Durham S K, Loy J K, Bravo R, Ransohoff R M, Lira S A (1996) Neutrophil infiltration, glial reaction, and neurological disease in transgenic mice expressing the chemokine N51/KC in oligodendrocytes. J. Clin. Invest. 98:529-39.
Toll, L., Berzetei-Gurske, I. P., Polgar, W. E., Brandt, S. R., Adapa, I. D., Rodriguez, L., et al. (1998). Standard binding and functional assays related to medications development division testing for potential cocaine and opiate narcotic treatment medications. NIDA Res Monogr 178, 440-466.
Tordo, J., C. O'Leary, A. Antunes, N. Palomar, P. Aldrin-Kirk, M. Basche, A. Bennett, Z. D'Souza, H. Gleitz, A. Godwin, R. J. Holley, H. Parker, A. Y. Liao, P. Rouse, A. S. Youshani, L. Dridi, C. Martins, T. Levade, K. B. Stacey, D. M. Davis, A. Dyer, N. Clement, T. Bjorklund, R. R. Ali, M. Agbandje-McKenna, A. A. Rahim, A. Pshezhetsky, S. N. Waddington, R. M. Linden, B. W. Bigger and E. Henckaerts (2018). “A novel adeno-associated virus capsid with enhanced neurotropism corrects a lysosomal transmembrane enzyme deficiency.” Brain 141(7): 2014-2031.
Tortella F C. Endogenous opioid peptides and epilepsy: Quieting the seizing brain? 1988. Trends Pharmacol Sci 9:366-372.
Wang Y, DeMayo F J, Tsai SY, O'Malley B W (1997) Ligand-inducible and liver-specific target gene expression in transgenic mice. Nat. Biotech. 15:239-43
Wang Y, Xu J, Pierson T, O'Malley B W, Tsai S Y (1997) Positive and negative regulation of gene expression in eucaryotic cells with an inducible transcriptional regulator. Gene Ther, 4:432-41.
Widmann M, Lieb A, Steck A, Fogli B, Mutti A, Schwarzer C (2022) doi: https://doi.org/10.1101/2022.07.05.498820
Xiao X, Li J, Samulski R J (1998) Production of high-titer recombinant adeno-associated virus vectors in the absence of helper adenovirus, J Virol 72(3) 2224-32
Zangrandi L, Burtscher J, MacKay J, Colmers W, & *Schwarzer C* (2016) The G-protein biased partial kappa opioid receptor agonist 6′-GNTI blocks hippocampal paroxysmal discharges without inducing aversion. British J Pharmacol, 173(11):1756-67,doi: 10.1111/bph.13475

### EXAMPLES

### Example 1

Shortening of the human ppDyn cDNA to enable packing into scAAV vectors. Some amino acids in the region between signal peptide and the region coding for active peptides can be removed. However, a sorting motif responsible for packing the propeptide into large dense core vesicles needs to be conserved. Alternatively, the entire part N-terminal to the region coding for active peptides can be replaced by a different signal peptide and sorting motif.

### Example 2

Production of Mature Dynorphins by Two scAAV Vector Variants

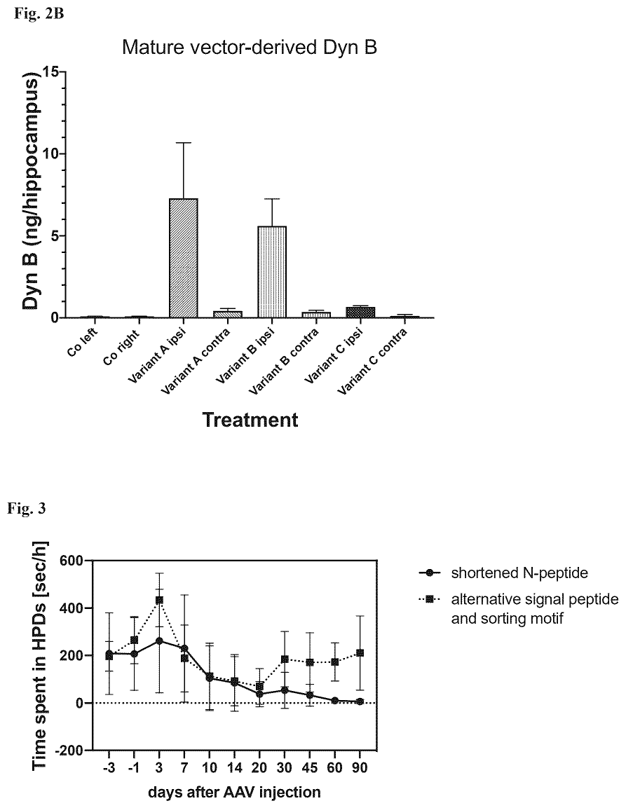

Production of mature dynorphins by three scAAV vector variants containing shortened pDyn cDNA. The vectors were injected into the dorsal hippocampus of naive wild-type mice. After 2 weeks the hippocampi were resected and the content of dynorphin A (DynA) and dynorphin B (DynB) was measured by ELISA as described in Agostinho et al. (2019), see FIG. 2. The production of mature peptides occurs only in large dense core vesicles, proving the correctness of sorting of the shortened pDyn variants A (SEQ ID No. 76) and B (SEQ ID No. 81). The marked reduction of mature dynorphins applying variant C (SEQ ID No 80), which lacks the proposed sorting motif, indicates the importance of the proposed sorting motif.

### Example 3

Seizure Suppression by Shortened pDyn cDNA

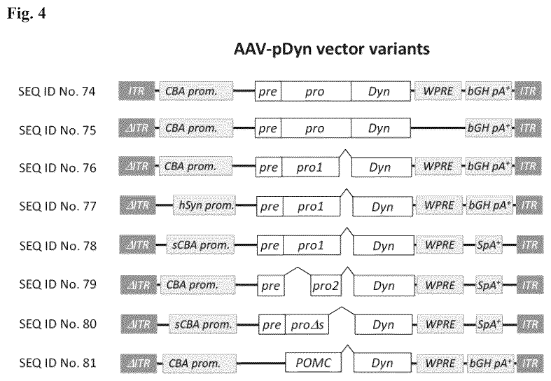

Seizure suppression by two scAAV vector variants containing shortened pDyn cDNA (FIG. 3). The vectors were injected into the dorsal hippocampus of epileptic wild-type mice. EEGs were recorded and analyzed for hippocampal paroxysmal discharges (HPD), representing drug-resistant focal seizures. The treatment of animals with kainic acid, the implantation of electrodes and the analysis is described in Widmann et al. (2022).

### Example 7

### Construction of AAV-pDyn Vector Variants

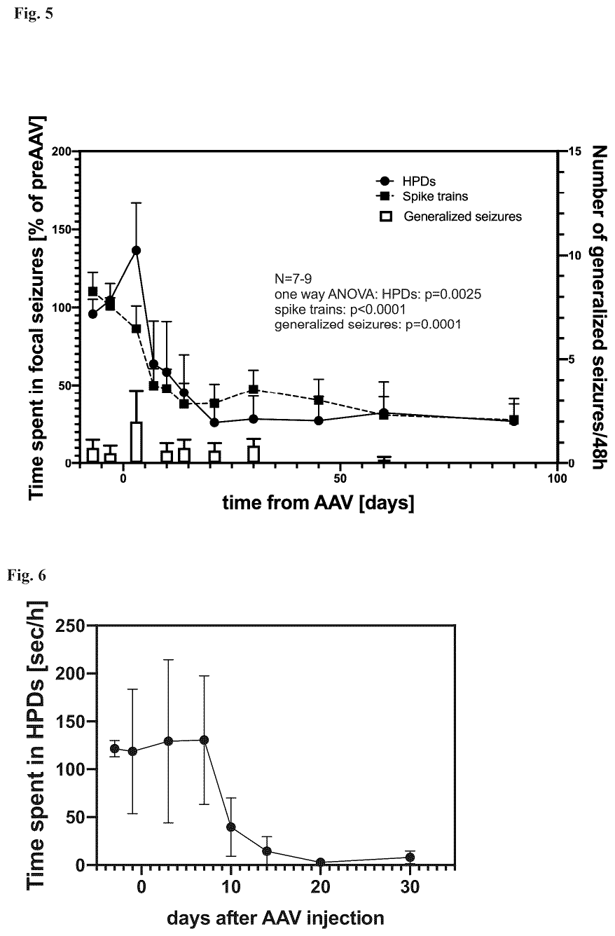

AAV vectors were constructed in the ssAAV or scAAV format as displayed in FIG. 4. They are composed of one AAV serotype 2 derived left and one right ITR sequence (SEQ ID No. 59 to 61). AAV ITRs of alternative AAV serotypes or synthetic ITRs may be used similarly.

The ITRs flank any of displayed heterologous gene expression cassettes (FIG. 4). These cassettes are composed of one of the promoter sequences (Seq ID No. 63 to 65), a posttranscriptional regulatory element from woodchuck hepatitis virus (Seq ID No. 66) (as described in Loeb et al. Hum Gene Ther 10:2295-2305, 1999), a polyadenylation signal, either derived from the bovine growth hormone gene (SEQ ID No. 67) or a short synthetic polyA signal sequence (SEQ ID No. 68) (as described in Levitt et al. Genes & Dev 3:1019-25, 1989) and the cDNA to be expressed.

The gene of interest is the cDNA sequence of human preprodynorphin (ppDyn), any of the displayed variants thereof (SEQ ID No. 69 to 72), or a fusion of the N-terminus of POMC with the C-terminal part of prodynorphin devoid of its signal-sequence (pre) and N-peptide (pro) (SEQ ID No. 73).

The cDNAs are codon-optimized in two versions. Version 1 (SEQ ID No. 69) is contained in AAV SEQ ID No. 74 and SEQ ID No. 75. Codon optimized version 2 ((SEQ ID No. 70 to 73) was generated to reduce the percentage of CpG sequence elements. Unmethylated CpG sequence elements are hallmarks of bacterial DNA and represent pathogen-associated molecular patterns (PAMP) which may activate the innate immune system in a mammalian host and could be an issue in the context of AAV gene therapies under certain circumstances. High CpG content of transduced AAV genomes may be associated with an increased probability of immune-mediated loss of transduced cells. Adverse effects of high CpG content of transduced AAV genomes have recently been shown also in the CNS after intraparenchymal/intracerebral AAV transduction (Suriano et al. 2021). Alternative codon optimization strategies to combine enhanced transgene expression in human cells with sufficient reduction of AAV-transduced unmethylated CpG elements may be achieved by different DNA sequence alterations.

The complete AAV DNA sequences from ITR to ITR spanning all regulator elements and the different ppDyn-derived transgenes as displayed in FIG. 4 and are represented as DNA sequence files (SEQ ID No. 74 to 81).

Any of the elements may be further interchanged, e.g. the truncated ITR of scAAV may be positioned at the right instead of the left end of the AAV genome as displayed here. Likewise, the gene promoters may be interchanged and/or combined with any of the displayed poly A signal sequence, or a polyA signal of different origin. The WPRE element may be used as full-length 582 bp element (Seq ID 66) as displayed. WPRE is composed of subelements named gamma, alpha, beta, in the given order. Shorter version (WPRE2) displaying a minimal gamma and partial alpha/beta element and WPRE3 displaying only minimal gamma and alpha elements (247 bp) were described to be similarly active (Choi et al. 2014, incorporated herein by reference). The versions of WPRE may be used interchangeably or may not be incorporated into the AAV genome at all.

### Example 8

### Functional Testing of AAV Vectors

AAV vectors of SEQ ID No. 76 to 78 and SEQ ID No. 81 were tested functionally. Fully processed, mature dynorphin peptides were produced (Seq ID No. 76 and No. 81; Example 3) and suppression of focal seizures were monitored in the TLE mouse model SEQ ID No. 76, 77, 78).

The reduction of distinct types of seizure activity after injection of AAV-pDyn expressing Seq ID No. 70. Hpds are shown in FIG. 5. Hpds, generalized seizures and spike trains were measured over periods of 48 hours each time-interval. The mice show a significant reduction of drug-resistant focal seizures starting from 7 days after treatment. Generalized seizures are almost completely abolished after 4 weeks.

The reduction of HPDs after injection of AAV-pDyn expressing Seq ID No. 77 is shown in FIG. 6. The mice show a marked reduction of drug-resistant focal seizures starting from 10 days after AAV delivery.

1. A delivery vector comprising a DNA sequence encoding pre-prodynorphin or pre-prodynorphin-variants and
wherein said delivery vector drives expression of a pre-propeptide in a target cell, and
wherein said delivery vector comprising said DNA sequence enables the release of dynorphin or dynorphin-variants from the target cell on demand, and
wherein said pre-propeptide is pre-prodynorphin or a pre-prodynorphin-variant and
wherein said pre-propeptide comprises a signal peptide, wherein the signal peptide is a N-terminal extension of a nascent polypeptide chain and wherein said signal peptide mediates protein targeting to the lumen of the endoplasmatic reticulum, and,
wherein said pre-propeptide comprises either (i) a N-terminal pro-peptide fragment on the C-terminal end of said signal peptide and wherein said N-terminal pro-peptide fragment comprises a sorting motif comprising the elements DL and EXyL, in particular a sorting motif consisting of the amino acid sequence DLXxEXyL (SEQ ID No. 36), where x is an integer from 1 to 20, y is an integer from 1 to 10, and each instance of X may independently be any amino acid (for the avoidance of doubt, this means that in the present invention in the first sequence of e.g. 1 to 20 X, each X may individually be any amino acid, and in the second sequence of e.g. 1 to 10 X, each X may individually be any amino acid, particularly as further defined herein), or wherein said pre-propeptide comprises (ii) a N-terminal pro-peptide fragment of a pre-pro-neuropeptide or protein sorted to large dense core vesicles, other than pre-prodynorphin, on the C-terminal end of said signal peptide and wherein said N-terminal pro-peptide fragment comprises a sorting motif of said pre-pro-neuropeptide or protein sorted to large dense core vesicles, and wherein said N-terminal pro-peptide fragment consists of 16 to 90 amino acids, and
wherein said pre-prodynorphin or pre-prodynorphin-variants comprise at least one of the following sequences selected from the group:
a. Dyn A that is SEQ ID No. 2 or a variant thereof consisting of the first 13 amino acids from the N-terminal end or a variant thereof consisting of the first 8 amino acids from the N-terminal end
b. Dyn B that is SEQ ID No. 3
c. leumorphin that is SEQ ID No. 4
d. variants of Dyn A, said variants having an amino acid sequence identity of at least 60% within the first 8 amino acids from the N-terminal end of SEQ ID No. 2,
e. variants of Dyn B, said variants having an amino acid sequence identity of at least 60% within the first 8 amino acids from the N-terminal end of SEQ ID No. 3,
f. variants of leumorphin, said variants having an amino acid sequence identity of at least 60% within the first 8 amino acids from the N-terminal end of SEQ ID No. 4.

2. A delivery vector comprising a DNA sequence encoding pre-prodynorphin or pre-prodynorphin-variants according to claim 1,
wherein said N-terminal pro-peptide fragment consists of 20 to 90 amino acids, preferably 30 and 90 amino acids;
or
wherein said N-terminal pro-peptide fragment on the C-terminal end of the signal peptide is a modified propeptide fragment of ppDyn wherein the unmodified propeptide fragment of ppDyn is
SEQ ID No. 5
DCLSRCSLCA VKTQDGPKPI NPLICSLQCQ AALLPSEEWE
RCQSFLSFFT PSTLGLNDKE DLGSKSVGEG PYSELAKLSG
SFLKELEKSK FLPSISTKEN TLSKSLEEKL RGLSDGFREG
AESELMRDAQ LNDGAMETGT LYLAEEDPKE QV
and wherein the modification of said propeptide fragment of ppDyn is a shortening:
or the modification is a replacement of parts of SEQ ID No. 5 with a propeptide of or a fragment of a propeptide of a neuropeptide:
wherein the modified propeptide fragment of ppDyn i) comprises at least one sorting motif comprising the elements DL and EXyL, in particular a sorting motif consisting of the amino acid sequence DLXxEXyL (SEQ ID No. 36) or ii) comprises a sorting motif of said pre-pro-neuropeptide or protein sorted to large dense core vesicles other than pre-prodynorphin;
or
wherein said N-terminal pro-peptide fragment on the C-terminal end of the signal peptide is a modified propeptide fragment of ppDyn that comprises or consists of
SEQ ID No. 6
DCLSRCSLCAVKTQDGPKPINPLICSLQCQAALLPSEEWERCQS
FLSFFTPSTLGLNDKEDLGSKSVGEGPYSELAKLSGSFLRKEQV
KR;
or
wherein said N-terminal pro-peptide fragment on the C-terminal end of the signal peptide is a modified propeptide fragment of ppDyn that comprises or consists of
SEQ ID No. 7
DLGSKSVGEG PYSELAKLSG SFLKELEKSK FLPSISTKEN
TLSKSLEEKL RGLSDGFREG AESELMRDAQ LNDGAMETGT
LYLAEEDPKE QV
or
SEQ ID No. 8
DLGSKSVGEG PYSELAKLSG SFLRKE QV
or
SEQ ID No 9
DLGSKSVGEG PYSELAKLRKE QV;
or
wherein the modified propeptide fragment is optionally flanked by peptidase recognition signals comprising K, R, KR, RK or RR;
or
wherein the target cells are neuronal cells of the central nervous system;
or
wherein the signal peptide is a peptide sequence of from 10 to 30 amino acids at the N-terminal end of precursor-proteins that are destined into the lumen of the endoplasmatic reticulum;
or
wherein the signal peptide is selected from the group comprising:
(SEQ ID No. 11)
MAWQGLVLAACLLMFPSTTA
(SEQ ID No. 12)
MARFLTLCTWLLLLGPGLLATVRA
(SEQ ID No. 13)
MLGNKRLGLSGLTLALSLLVCLGALAEA
(SEQ ID No. 14)
MLSCRLQCALAALSIVLALGCVTG
(SEQ ID No. 15)
MKILVALAVFFLVSTQLFA
(SEQ ID No. 16)
MRIMLLFTAILAFSLA
(SEQ ID No. 17)
MPRSCCSRSGALLLALLLQASMEVRG
(SEQ ID No. 18)
MNSGVCLCVLMAVLAAGA
(SEQ ID No. 19)
MKVLLCDLLLLSLFSSVFS
(SEQ ID NO. 20)
MQPTLLLSLLGAVGLAAVNS;
or
wherein said delivery vector leads to release-on-demand of dynorphins or dynorphin-variants with agonistic effects on human Kappa Opioid Receptors;
or
wherein the dynorphin variants have an amino acid sequence identity of at least 70% within the first 8 amino acids from the N-terminal end of SEQ ID No. 2 (YGGFLRRI), SEQ ID No. 3 (YGGFLRRQ) or SEQ ID No. 4 (YGGFLRRQ), respectively;
or
wherein the variants have an amino acid sequence identity of at least 80% within the first 8 amino acids from the N-terminal end of SEQ ID No. 2, SEQ ID No. 3 or SEQ ID No. 4, respectively;
wherein said delivery vector comprises in addition a recombinant adeno-associated virus (AAV) vector genome or a recombinant lentivirus genome;
or
comprising a recombinant adeno-associated virus (AAV) vector genome comprising inverted terminal repeats (ITR) preferably derived from AAV serotype 2, alternatively from AAV serotype 1, 3, 4, 5, 6, 7, 8, 9, 10, rh10, 11, 12, 13, 14, or reengineered variants thereof, wherein said vector genome is packaged in an AAV capsid selected from the group comprising AAV serotypes 1, 2, 3, 4, 5, 6, 7, 8, 9, 10, rh10, 11, 12, 13, 14, serpentine AAV, ancestral AAV, AAV-TT, AAVv66, AAV1P4, AAV1P5, AAV-PHP.B, AAV-PHP.eB, AAV2-HBKO, AAV.CAP-B10, AAV.CAP-MAC, AAV2.NN, or further AAV capsid mutants derived thereof, preferably of AAV serotype 1 or 2 or a chimeric vector comprising capsid proteins derived from two or more, preferably two of the aforementioned AAV serotype capsids; in particular embodiments, the capsids are capsids derived from AAV serotype 1 and/or 2.

3-5. (canceled)

6. A delivery vector comprising a DNA sequence encoding pre-prodynorphin or pre-prodynorphin-variants according to claim 1, wherein said N-terminal pro-peptide fragment on the C-terminal end of the signal peptide is a modified propeptide fragment of ppDyn wherein the unmodified propeptide fragment of ppDyn is
SEQ ID No. 5
DCLSRCSLCA VKTQDGPKPI NPLICSLQCQ AALLPSEEWE
RCQSFLSFFT PSTLGLNDKE DLGSKSVGEG PYSELAKLSG
SFLKELEKSK FLPSISTKEN TLSKSLEEKL RGLSDGFREG
AESELMRDAQ LNDGAMETGT LYLAEEDPKE QV
and wherein the modification of said propeptide fragment of ppDyn is a shortening;
or the modification is a replacement of parts of SEQ ID No. 5 with a propeptide of or a fragment of a propeptide of a neuropeptide;
wherein the modified propeptide fragment of ppDyn i) comprises at least one sorting motif comprising the elements DL and EXyL, in particular a sorting motif consisting of the amino acid sequence DLXxEXyL (SEQ ID No. 36) or ii) comprises a sorting motif of said pre-pro-neuropeptide or protein sorted to large dense core vesicles other than pre-prodynorphin;
wherein the modification is a replacement of parts of SEQ ID No. 5 with a propeptide of or a fragment of a propeptide of a neuropeptide; wherein the modified propeptide fragment of ppDyn i) comprises at least one sorting motif comprising the elements DL and EXyL, in particular a sorting motif consisting of the amino acid sequence DLXxEXyL (SEQ ID No. 36) or ii) comprises a sorting motif of said pre-pro-neuropeptide or protein sorted to large dense core vesicles other than pre-prodynorphin.

7. A delivery vector comprising a DNA sequence encoding pre-prodynorphin or pre-prodynorphin-variants according to claim 6
wherein the modified propeptide fragment comprises or consists of SEQ ID No. 10
MPRSCCSRSGALLLALLLQASMEVRGWCLESSQCQDLTTESNLLEC
IRACKP;
or
wherein the modified propeptide fragment comprises or consists of a propeptide fragment of preproEnkephalin, preproBDNF, preproTachykinin, prepro-Somatostatin, pre-pro-VIP, prepro-CCK, preproNociceptin or preproNPY, wherein said propeptide fragment comprises said sorting motif, in particularly said propeptide fragment maybe selected from the group comprising of any of SEQ ID Nos: 37 to 52.

8-17. (canceled)

18. A delivery vector comprising a DNA sequence encoding pre-prodynorphin or pre-prodynorphin-variants according to claim 1.

19. A recombinant virus particle or a liposome or nanoparticle, comprising a delivery vector according to claim 1.

20. The recombinant virus particle or liposome or nanoparticle of claim 19, wherein said delivery vector comprises in addition a recombinant adeno-associated virus (AAV) vector genome and said rAAV vector genome is encapsidated in an AAV capsid or wherein said delivery vector comprises in addition a recombinant lentivirus vector genome and is packaged in a lentivirus particle.

21. A method for delivering a nucleic acid to a cell of the central nervous system, comprising contacting the cell with the delivery vector or recombinant virus particle according to claim 1 or a liposome or nanoparticle comprising said delivery vector under conditions sufficient for the DNA sequence encoding pre-prodynorphin or pre-prodynorphin-variants to be introduced into the cell.

22. A pharmaceutical composition comprising a carrier and a delivery vector according to claim 1 or a recombinant virus particle or liposome or nanoparticle comprising said delivery vector.

23. A method for treating focal epilepsy in a subject, in particular mesial temporal lobe epilepsy, or for preventing epileptic seizures in a subject that suffers from focal epilepsy whereby said delivery vector or recombinant virus particle or liposome or nanoparticle provides activation of human Kappa Opioid Receptors in the epileptogenic focus, thereby inhibiting seizures, comprising administering to said subject a pharmaceutical composition according to claim 22.

24. A DNA sequence selected from the following sequences: SEQ ID No. 69, SEQ ID No. 70, SEQ ID No. 71, SEQ ID No. 72, and SEQ ID No. 73.

25. A delivery vector comprising a DNA sequence according to claim 24.

26. A delivery vector according to claim 25,
wherein said delivery vector comprises in addition a recombinant adeno-associated virus (AAV) vector genome or a recombinant lentivirus genome;
or
comprising a recombinant adeno-associated virus (AAV) vector genome comprising inverted terminal repeats (ITR), preferably derived from AAV serotype 2, alternatively from AAV serotype 1, 3, 4, 5, 6, 7, 8, 9, 10, rh10, 11, 12, 13, 14, or reengineered variants thereof, wherein said vector genome is packaged in an AAV capsid selected from the group comprising AAV serotypes 1, 2, 3, 4, 5, 6, 7, 8, 9, 10, rh10, 11, 12, 13, 14, serpentine AAV, ancestral AAV, AAV-TT, AAVv66, AAV1P4, AAV1P5, AAV-PHP.B, AAV-PHP.eB, AAV2-HBKO, AAV.CAP-B10, AAV.CAP-MAC, AAV2.NN, or further AAV capsid mutants derived thereof, or a chimeric vector comprising mosaic capsid proteins derived from two or more, preferably two of the aforementioned AAV serotype capsids; in particular embodiments, the capsids are capsids derived from AAV serotype 1 and/or 2;
or
wherein said delivery vector comprises in addition at least one sequence selected from the group comprising SEQ ID No. 59, SEQ ID No. 60, SEQ ID No. 61, SEQ ID No. 63, SEQ ID No. 64, SEQ ID No. 65, SEQ ID No. 66, SEQ ID No. 67, and SEQ ID No. 68;
or
wherein said delivery vector comprises a sequence selected from the group comprising SEQ ID No. 74, SEQ ID No. 75, SEQ ID No. 76, SEQ ID No. 77, SEQ ID No. 78, SEQ ID No. 79, SEQ ID No. 80, and SEQ ID No. 81.

27-29. (canceled)

30. A recombinant virus particle or a liposome or nanoparticle, comprising a delivery vector according to claim 25.

31. The recombinant virus particle or liposome or nanoparticle according to claim 30, wherein said delivery vector comprises in addition a recombinant adeno-associated virus (AAV) vector genome and said rAAV vector genome is encapsidated in an AAV capsid or wherein said delivery vector comprises in addition a recombinant lentivirus vector genome and is packaged in a lentivirus particle.

32. A method for delivering a nucleic acid to a cell of the central nervous system, comprising contacting the cell with the delivery vector according to claim 25 or with a recombinant virus particle or liposome or nanoparticle comprising said delivery vector under conditions sufficient for a DNA comprising a sequence selected from the group comprising the SEQ ID No. 69, SEQ ID No. 70, SEQ ID No. 71, SEQ ID No. 72, and SEQ ID No. 73 to be introduced into the cell.

33. A pharmaceutical composition comprising a carrier and a delivery vector according to claim 25, or a recombinant virus particle or liposome or nanoparticle comprising said delivery vector.

34. A method for treating epilepsy in a subject, in particular focal epilepsy, in particular mesial temporal lobe epilepsy, or for preventing epileptic seizures in a subject that suffers from focal epilepsy, wherein said delivery vector or recombinant virus particle or liposome or nanoparticle provides activation of human Kappa Opioid Receptors in the epileptogenic focus, thereby inhibiting seizures, comprising administering to said subject a pharmaceutical composition according to claim 33.


SEQUENCE LISTING (81 sequences)

SEQ ID NO: 1 - protein, 254 aa, Homo sapiens [sequence omitted]

SEQ ID NO: 2 - protein, 17 aa, Homo sapiens
YGGFLRRIRPKLKWDNQ

SEQ ID NO: 3 - protein, 13 aa, Homo sapiens
YGGFLRRQFKVVT

SEQ ID NO: 4 - protein, 29 aa, Homo sapiens
YGGFLRRQFKVVTRSQEDPNAYSGELFDA

SEQ ID NO: 5 - protein, 152 aa, Homo sapiens
DCLSRCSLCAVKTQDGPKPINPLICSLQCQAALLPSEEWERCQSFLSFFTPSTLGLNDKEDLGSKSVGEGPYSELAKLSGSFLKELEKSKFLPSISTKENTLSKSLEEKLRGLSDGFREGAESELMRDAQLNDGAMETGTLYLAEEDPKEQV

SEQ ID NO: 6 - protein, 90 aa, synthetic construct
DCLSRCSLCAVKTQDGPKPINPLICSLQCQAALLPSEEWERCQSFLSFFTPSTLGLNDKEDLGSKSVGEGPYSELAKLSGSFLRKEQVKR

SEQ ID NO: 7 - protein, 92 aa, synthetic construct
DLGSKSVGEGPYSELAKLSGSFLKELEKSKFLPSISTKENTLSKSLEEKLRGLSDGFREGAESELMRDAQLNDGAMETGTLYLAEEDPKEQV

SEQ ID NO: 8 - protein, 28 aa, synthetic construct
DLGSKSVGEGPYSELAKLSGSFLRKEQV

SEQ ID NO: 9 - protein, 23 aa, synthetic construct
DLGSKSVGEGPYSELAKLRKEQV

SEQ ID NO: 10 - protein, 52 aa, Homo sapiens
MPRSCCSRSGALLLALLLQASMEVRGWCLESSQCQDLTTESNLLECIRACKP

SEQ ID NO: 11 - protein, 20 aa, Homo sapiens
MAWQGLVLAACLLMFPSTTA

SEQ ID NO: 12 - protein, 24 aa, Homo sapiens
MARFLTLCTWLLLLGPGLLATVRA

SEQ ID NO: 13 - protein, 28 aa, Homo sapiens
MLGNKRLGLSGLTLALSLLVCLGALAEA

SEQ ID NO: 14 - protein, 24 aa, Homo sapiens
MLSCRLQCALAALSIVLALGCVTG

SEQ ID NO: 15 - protein, 19 aa, Homo sapiens
MKILVALAVFFLVSTQLFA

SEQ ID NO: 16 - protein, 16 aa, Homo sapiens
MRIMLLFTAILAFSLA

SEQ ID NO: 17 - protein, 26 aa, Homo sapiens
MPRSCCSRSGALLLALLLQASMEVRG

SEQ ID NO: 18 - protein, 18 aa, Homo sapiens
MNSGVCLCVLMAVLAAGA

SEQ ID NO: 19 - protein, 19 aa, Homo sapiens
MKVLLCDLLLLSLFSSVFS

SEQ ID NO: 20 - protein, 20 aa, Homo sapiens
MQPTLLLSLLGAVGLAAVNS

SEQ ID NO: 21-35 - skipped (15 entries)

SEQ ID NO: 36 - protein, 6 aa, synthetic construct
DLXEXL

SEQ ID NO: 37 - protein, 97 aa, Homo sapiens
MARFLTLCTWLLLLGPGLLATVRAECSQDCATCSYRLVRPADINFLACVMECEGKLPSLKIWETCKELLQLSKPELPQDGTSTLRENSKPEESHLLA

SEQ ID NO: 38 - protein, 179 aa, synthetic construct
MARFLTLCTWLLLLGPGLLATVRAECSQDCATCSYRLVRPADINFLACVMECEGKLPSLKIWETCKELLQLSKPELPQDGTSTLRENSKPEESHLLAKRYGGFLRKYPKRSSEVAGEGDGDSMGHEDLYKRYGGFLRRIRPKLKWDNQKRYGGFLRRQFKVVTRSQEDPNAYSGELFDA

SEQ ID NO: 39 - protein, 28 aa, Homo sapiens
MLGNKRLGLSGLTLALSLLVCLGALAEA

SEQ ID NO: 40 - protein, 109 aa, synthetic construct
MLGNKRLGLSGLTLALSLLVCLGALAEAKRYGGFLRKYPKRSSEVAGEGDGDSMGHEDLYKRYGGFLRRIRPKLKWDNQKRYGGFLRRQFKVVTRSQEDPNAYSGELFD

SEQ ID NO: 41 - protein, 124 aa, Homo sapiens
MTILFLTMVISYFGCMKAAPMKEANIRGQGGLAYPGVRTHGTLESVNGPKAGSRGLTSLADTFEHVIEELLDEDHKVRPNEENNKDADLYTSRVMLSSQVPLEPPLLFLLEEYKNYLDAANMSM

SEQ ID NO: 42 - protein, 205 aa, synthetic construct
MTILFLTMVISYFGCMKAAPMKEANIRGQGGLAYPGVRTHGTLESVNGPKAGSRGLTSLADTFEHVIEELLDEDHKVRPNEENNKDADLYTSRVMLSSQVPLEPPLLFLLEEYKNYLDAANMSMKRYGGFLRKYPKRSSEVAGEGDGDSMGHEDLYKRYGGFLRRIRPKLKWDNQKRYGGFLRRQFKVVTRSQEDPNAYSGELFD

SEQ ID NO: 43 - protein, 88 aa, Homo sapiens
MLSCRLQCALAALSIVLALGCVTGAPSDPRLRQFLQKSLAAAAGKQELAKYFLAELLSEPNQTENDALEPEDLSQAAEQDEMRLELQR

SEQ ID NO: 44 - protein, 169 aa, synthetic construct
MLSCRLQCALAALSIVLALGCVTGAPSDPRLRQFLQKSLAAAAGKQELAKYFLAELLSEPNQTENDALEPEDLSQAAEQDEMRLELQRKRYGGFLRKYPKRSSEVAGEGDGDSMGHEDLYKRYGGFLRRIRPKLKWDNQKRYGGFLRRQFKVVTRSQEDPNAYSGELFD

SEQ ID NO: 45 - protein, 56 aa, Homo sapiens
MKILVALAVFFLVSTQLFAEEIGANDDLNYWSDWYDSDQIKEELPEPFEHLLQRIA

SEQ ID NO: 46 - protein, 137 aa, synthetic construct
MKILVALAVFFLVSTQLFAEEIGANDDLNYWSDWYDSDQIKEELPEPFEHLLQRIAKRYGGFLRKYPKRSSEVAGEGDGDSMGHEDLYKRYGGFLRRIRPKLKWDNQKRYGGFLRRQFKVVTRSQEDPNAYSGELFD

SEQ ID NO: 47 - protein, 79 aa, Homo sapiens
MDTRNKAQLLVLLTLLSVLFSQTSAWPLYRAPSALRLGDRIPFEGANEPDQVSLKEDIDMLQNALAENDTPYYDVSRNA

SEQ ID NO: 48 - protein, 160 aa, synthetic construct
MDTRNKAQLLVLLTLLSVLFSQTSAWPLYRAPSALRLGDRIPFEGANEPDQVSLKEDIDMLQNALAENDTPYYDVSRNAKRYGGFLRKYPKRSSEVAGEGDGDSMGHEDLYKRYGGFLRRIRPKLKWDNQKRYGGFLRRQFKVVTRSQEDPNAYSGELFD

SEQ ID NO: 49 - protein, 44 aa, Homo sapiens
MNSGVCLCVLMAVLAAGALTQPVPPADPAGSGLQRAEEAPRRQL

SEQ ID NO: 50 - protein, 125 aa, synthetic construct
MNSGVCLCVLMAVLAAGALTQPVPPADPAGSGLQRAEEAPRRQLKRYGGFLRKYPKRSSEVAGEGDGDSMGHEDLYKRYGGFLRRIRPKLKWDNQKRYGGFLRRQFKVVTRSQEDPNAYSGELFD

SEQ ID NO: 51 - protein, 95 aa, Homo sapiens
MKVLLCDLLLLSLFSSVFSSCQRDCLTCQEKLHPALDSFDLEVCILECEEKVFPSPLWTPCTKVMARSSWQLSPAAPEHVAAALYQPRASEMQHL

SEQ ID NO: 52 - protein, 176 aa, synthetic construct
MKVLLCDLLLLSLFSSVFSSCQRDCLTCQEKLHPALDSFDLEVCILECEEKVFPSPLWTPCTKVMARSSWQLSPAAPEHVAAALYQPRASEMQHLKRYGGFLRKYPKRSSEVAGEGDGDSMGHEDLYKRYGGFLRRIRPKLKWDNQKRYGGFLRRQFKVVTRSQEDPNAYSGELFD

SEQ ID NO: 53 - protein, 190 aa, synthetic construct
MAWQGLVLAACLLMFPSTTADCLSRCSLCAVKTQDGPKPINPLICSLQCQAALLPSEEWERCQSFLSFFTPSTLGLNDKEDLGSKSVGEGPYSELAKLSGSFLRKEQVKRYGGFLRKYPKRSSEVAGEGDGDSMGHEDLYKRYGGFLRRIRPKLKWDNQKRYGGFLRRQFKVVTRSQEDPNAYSGELFDA

SEQ ID NO: 54 - protein, 138 aa, synthetic construct
MPRSCCSRSGALLLALLLQASMEVRGWCLESSQCQDLTTESNLLECIRACKPKEQVKRYGGFLRKYPKRSSEVAGEGDGDSMGHEDLYKRYGGFLRRIRPKLKWDNQKRYGGFLRRQFKVVTRSQEDPNAYSGELFDA

SEQ ID NO: 55 - protein, 20 aa, synthetic construct
DLGSKSVGEGPYSELRKEQV

SEQ ID NO: 56 - protein, 28 aa, synthetic construct
DLGSKSVGEGPYSELAKLSGSFLKKEQV

SEQ ID NO: 57 - protein, 18 aa, synthetic construct
DLGSKSVGEGPYSELAKL

SEQ ID NO: 58 - protein, 15 aa, synthetic construct
DLGSKSVGEGPYSEL

SEQ ID NO: 59 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 60 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 61 - DNA, 121 bp, synthetic construct
  source 1..121: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 62 - skipped

SEQ ID NO: 63 - DNA, 887 bp, synthetic construct
  source 1..887: mol_type = other DNA
taatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctagagcctctgctaaccatgttcatgccttcttctttttcctaca

SEQ ID NO: 64 - DNA, 851 bp, synthetic construct
  source 1..851: mol_type = other DNA
ccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctagagcctctgctaaccatgttcatgccttcttctttttcctaca

SEQ ID NO: 65 - DNA, 448 bp, Homo sapiens
  source 1..448: mol_type = genomic DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 66 - DNA, 582 bp, Woodchuck hepatitis virus
  source 1..582: mol_type = genomic DNA
ctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 67 - DNA, 208 bp, Bos taurus
  source 1..208: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 68 - DNA, 49 bp, synthetic construct
  source 1..49: mol_type = other DNA
aataaaagatctttattttcattagatctgtgtgttggttttttgtgtg

SEQ ID NO: 69 - DNA, 765 bp, synthetic construct
  source 1..765: mol_type = other DNA
atggcttggcagggactggtgctggctgcctgcctgctgatgttccctagcaccaccgccgactgcctgagcagatgtagcctgtgcgccgtgaaaacccaggacggccccaagcccatcaaccccctgatctgcagcctgcagtgtcaggccgctctgctgcctagcgaggaatgggagagatgccagagcttcctgagcttcttcacccccagcaccctgggcctgaacgacaaagaggacctgggcagcaagagcgtgggcgagggcccttattctgagctggccaagctgagcggcagctttctgaaagagctggaaaagagcaagttcctgcccagcatcagcaccaaagagaacaccctgagcaagagcctggaagagaagctgcggggcctgagcgacggctttagagagggcgccgagagcgagctgatgagggacgcccagctgaatgacggcgccatggaaaccggcaccctgtacctggccgaagaggaccccaaagaacaagtgaagagatacggcggcttcctgcggaagtaccccaagagatctagcgaggtggccggcgaaggcgacggcgattctatgggacacgaggacctgtacaagcgctatggcggctttctgaggcggatccggcccaagctgaagtgggacaaccagaaacgctacgggggattcctgaggcggcagttcaaggtcgtgacccggtcccaggaagatcccaacgcctacagcggcgagctgttcgacgcttga

SEQ ID NO: 70 - DNA, 576 bp, synthetic construct
  source 1..576: mol_type = other DNA
accatggcttggcagggacttgtgcttgcggcttgtctcctcatgttccctagtacaactgctgattgcctgagcagatgtagcctgtgcgccgtgaaaacccaggacggccccaagcccatcaaccccctgatctgcagcctgcagtgtcaggccgctctgctgcctagcgaggaatgggagagatgccagagcttcctgagcttcttcacccccagcaccctgggcctgaacgacaaagaggacctgggcagcaagagtgtaggtgaaggcccttactctgagttggcaaaactgtctggcagttttctgaggaaggaacaggtcaagcgatatggtggcttcctgagaaagtatccaaagagaagctcagaagttgcaggagaaggtgatggggatagtatgggacacgaagacctgtacaagcgctatggaggctttctgaggagaatacgtcccaaactgaaatgggacaaccagaaacgatatggaggcttcttgagaaggcagttcaaagtggtgactagaagccaagaggaccctaatgcctactcaggggagttgtttgatgcctga

SEQ ID NO: 71 - DNA, 399 bp, synthetic construct
  source 1..399: mol_type = other DNA
atggcttggcagggacttgtgcttgcggcttgtctcctcatgttccctagtacaactgctaaagaggacctgggcagcaagagtgtaggtgaaggcccttactctgagttggcaaaactgtctggcagttttctgaggaaggaacaggtcaagcgatatggtggcttcctgagaaagtatccaaagagaagctcagaagttgcaggagaaggtgatggggatagtatgggacacgaagacctgtacaagcgctatggaggctttctgaggagaatacgtcccaaactgaaatgggacaaccagaaacgatatggaggcttcttgagaaggcagttcaaagtggtgactagaagccaagaggaccctaatgcctactcaggggagttgtttgatgcctga

SEQ ID NO: 72 - DNA, 519 bp, synthetic construct
  source 1..519: mol_type = other DNA
atggcttggcagggacttgtgcttgcggcttgtctcctcatgttccctagtacaactgctgattgcctgagcagatgtagcctgtgcgccgtgaaaacccaggacggccccaagcccatcaaccccctgatctgcagcctgcagtgtcaggccgctctgctgcctagcgaggaatgggagagatgccagagcttcctgagcttcttcacccccagcaccctgggcctgaacgacaaagagtctggcagttttctgaggaaggaacaggtcaagcgatatggtggcttcctgagaaagtatccaaagagaagctcagaagttgcaggagaaggtgatggggatagtatgggacacgaagacctgtacaagcgctatggaggctttctgaggagaatacgtcccaaactgaaatgggacaaccagaaacgatatggaggcttcttgagaaggcagttcaaagtggtgactagaagccaagaggaccctaatgcctactcaggggagttgtttgatgcctga

SEQ ID NO: 73 - DNA, 417 bp, synthetic construct
  source 1..417: mol_type = other DNA
atgccaagatcatgctgttcacgtagtggagcccttctcttggcacttcttctgcaagcaagcatggaagtcagaggttggtgtttggagagttcccaatgccaagacctgactactgagtcaaacctgctggaatgcataagagcctgtaaacccaaggaacaggtgaaacgctatggtgggtttctcaggaaatatcccaaaagatcctctgaggttgctggtgaaggtgatggggacagtatgggccatgaggacctgtacaagagatatggtggattcctcagaagaatcagacccaaactgaagtgggacaaccagaagagatatggagggtttctgaggagacagttcaaggtagtcaccagatcccaagaggaccctaacgcgtatagtggggaactctttgatgcatga

SEQ ID NO: 74 - DNA, 2820 bp, synthetic construct
  source 1..2820: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctaattcgatatcaagcttatcgataccgtcgacctcgagggggtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttaaagggctccgggagggccctagagcctctgctaaccatgttcatgccttcttctttttcctacagcggccgcgccaccatggcttggcagggactggtgctggctgcctgcctgctgatgttccctagcaccaccgccgactgcctgagcagatgtagcctgtgcgccgtgaaaacccaggacggccccaagcccatcaaccccctgatctgcagcctgcagtgtcaggccgctctgctgcctagcgaggaatgggagagatgccagagcttcctgagcttcttcacccccagcaccctgggcctgaacgacaaagaggacctgggcagcaagagcgtgggcgagggcccttattctgagctggccaagctgagcggcagctttctgaaagagctggaaaagagcaagttcctgcccagcatcagcaccaaagagaacaccctgagcaagagcctggaagagaagctgcggggcctgagcgacggctttagagagggcgccgagagcgagctgatgagggacgcccagctgaatgacggcgccatggaaaccggcaccctgtacctggccgaagaggaccccaaagaacaagtgaagagatacggcggcttcctgcggaagtaccccaagagatctagcgaggtggccggcgaaggcgacggcgattctatgggacacgaggacctgtacaagcgctatggcggctttctgaggcggatccggcccaagctgaagtgggacaaccagaaacgctacgggggattcctgaggcggcagttcaaggtcgtgacccggtcccaggaagatcccaacgcctacagcggcgagctgttcgacgcttgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagaagcttacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 75 - DNA, 2219 bp, synthetic construct
  source 1..2219: mol_type = other DNA
ccactccctctctgcgcgctcgctcgctcactgaggccgggcgaccaaaggtcgcccgacgcccgggctttgcccgggcggcctcagtgagcgagcgagcgcgcagagagggagtggaattcgatatcaagcttatcgataccgtcgacctcgagggggtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctagagcctctgctaaccatgttcatgccttcttctttttcctacagcggccgcgccaccatggcttggcagggactggtgctggctgcctgcctgctgatgttccctagcaccaccgccgactgcctgagcagatgtagcctgtgcgccgtgaaaacccaggacggccccaagcccatcaaccccctgatctgcagcctgcagtgtcaggccgctctgctgcctagcgaggaatgggagagatgccagagcttcctgagcttcttcacccccagcaccctgggcctgaacgacaaagaggacctgggcagcaagagcgtgggcgagggcccttattctgagctggccaagctgagcggcagctttctgaaagagctggaaaagagcaagttcctgcccagcatcagcaccaaagagaacaccctgagcaagagcctggaagagaagctgcggggcctgagcgacggctttagagagggcgccgagagcgagctgatgagggacgcccagctgaatgacggcgccatggaaaccggcaccctgtacctggccgaagaggaccccaaagaacaagtgaagagatacggcggcttcctgcggaagtaccccaagagatctagcgaggtggccggcgaaggcgacggcgattctatgggacacgaggacctgtacaagcgctatggcggctttctgaggcggatccggcccaagctgaagtgggacaaccagaaacgctacgggggattcctgaggcggcagttcaaggtcgtgacccggtcccaggaagatcccaacgcctacagcggcgagctgttcgacgcttgagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 76 - DNA, 2603 bp, synthetic construct
  source 1..2603: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggattcgatatcaagcttatcgataccgtcgacctcgagggggtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctagagcctctgctaaccatgttcatgccttcttctttttcctacagcggccgcgccaccatggcttggcagggacttgtgcttgcggcttgtctcctcatgttccctagtacaactgctgattgcctgagcagatgtagcctgtgcgccgtgaaaacccaggacggccccaagcccatcaaccccctgatctgcagcctgcagtgtcaggccgctctgctgcctagcgaggaatgggagagatgccagagcttcctgagcttcttcacccccagcaccctgggcctgaacgacaaagaggacctgggcagcaagagtgtaggtgaaggcccttactctgagttggcaaaactgtctggcagttttctgaggaaggaacaggtcaagcgatatggtggcttcctgagaaagtatccaaagagaagctcagaagttgcaggagaaggtgatggggatagtatgggacacgaagacctgtacaagcgctatggaggctttctgaggagaatacgtcccaaactgaaatgggacaaccagaaacgatatggaggcttcttgagaaggcagttcaaagtggtgactagaagccaagaggaccctaatgcctactcaggggagttgtttgatgcctgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagaagcttacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 77 - DNA, 2164 bp, synthetic construct
  source 1..2164: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggattcgatatcaagcttatcgataccgtcgacctcgagggggagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggcggccgcgccaccatggcttggcagggacttgtgcttgcggcttgtctcctcatgttccctagtacaactgctgattgcctgagcagatgtagcctgtgcgccgtgaaaacccaggacggccccaagcccatcaaccccctgatctgcagcctgcagtgtcaggccgctctgctgcctagcgaggaatgggagagatgccagagcttcctgagcttcttcacccccagcaccctgggcctgaacgacaaagaggacctgggcagcaagagtgtaggtgaaggcccttactctgagttggcaaaactgtctggcagttttctgaggaaggaacaggtcaagcgatatggtggcttcctgagaaagtatccaaagagaagctcagaagttgcaggagaaggtgatggggatagtatgggacacgaagacctgtacaagcgctatggaggctttctgaggagaatacgtcccaaactgaaatgggacaaccagaaacgatatggaggcttcttgagaaggcagttcaaagtggtgactagaagccaagaggaccctaatgcctactcaggggagttgtttgatgcctgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagaagcttacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 78 - DNA, 2388 bp, synthetic construct
  source 1..2388: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggattcgatatcatcgataccgtccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctagagcctctgctaaccatgttcatgccttcttctttttcctacagcggccgcgccaccatggcttggcagggacttgtgcttgcggcttgtctcctcatgttccctagtacaactgctgattgcctgagcagatgtagcctgtgcgccgtgaaaacccaggacggccccaagcccatcaaccccctgatctgcagcctgcagtgtcaggccgctctgctgcctagcgaggaatgggagagatgccagagcttcctgagcttcttcacccccagcaccctgggcctgaacgacaaagaggacctgggcagcaagagtgtaggtgaaggcccttactctgagttggcaaaactgtctggcagttttctgaggaaggaacaggtcaagcgatatggtggcttcctgagaaagtatccaaagagaagctcagaagttgcaggagaaggtgatggggatagtatgggacacgaagacctgtacaagcgctatggaggctttctgaggagaatacgtcccaaactgaaatgggacaaccagaaacgatatggaggcttcttgagaaggcagttcaaagtggtgactagaagccaagaggaccctaatgcctactcaggggagttgtttgatgcctgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcagcctcgaaataaaagatctttattttcattagatctgtgtgttggttttttgtgtggagaagcttacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 79 - DNA, 2270 bp, synthetic construct
  source 1..2270: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggattcgatatcaagcttatcgataccgtcgacctcgagggggtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctagagcctctgctaaccatgttcatgccttcttctttttcctacagcggccgcgccaccatggcttggcagggacttgtgcttgcggcttgtctcctcatgttccctagtacaactgctaaagaggacctgggcagcaagagtgtaggtgaaggcccttactctgagttggcaaaactgtctggcagttttctgaggaaggaacaggtcaagcgatatggtggcttcctgagaaagtatccaaagagaagctcagaagttgcaggagaaggtgatggggatagtatgggacacgaagacctgtacaagcgctatggaggctttctgaggagaatacgtcccaaactgaaatgggacaaccagaaacgatatggaggcttcttgagaaggcagttcaaagtggtgactagaagccaagaggaccctaatgcctactcaggggagttgtttgatgcctgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcagcctcgaaataaaagatctttattttcattagatctgtgtgttggttttttgtgtggagaagcttacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 80 - DNA, 2334 bp, synthetic construct
  source 1..2334: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggattcgatatcatcgataccgtccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctagagcctctgctaaccatgttcatgccttcttctttttcctacagcggccgcgccaccatggcttggcagggacttgtgcttgcggcttgtctcctcatgttccctagtacaactgctgattgcctgagcagatgtagcctgtgcgccgtgaaaacccaggacggccccaagcccatcaaccccctgatctgcagcctgcagtgtcaggccgctctgctgcctagcgaggaatgggagagatgccagagcttcctgagcttcttcacccccagcaccctgggcctgaacgacaaagagtctggcagttttctgaggaaggaacaggtcaagcgatatggtggcttcctgagaaagtatccaaagagaagctcagaagttgcaggagaaggtgatggggatagtatgggacacgaagacctgtacaagcgctatggaggctttctgaggagaatacgtcccaaactgaaatgggacaaccagaaacgatatggaggcttcttgagaaggcagttcaaagtggtgactagaagccaagaggaccctaatgcctactcaggggagttgtttgatgcctgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcagcctcgaaataaaagatctttattttcattagatctgtgtgttggttttttgtgtggagaagcttacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 81 - DNA, 2447 bp, synthetic construct
  source 1..2447: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggattcgatatcaagcttatcgataccgtcgacctcgagggggtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctagagcctctgctaaccatgttcatgccttcttctttttcctacagcggccgcgccaccatgccaagatcatgctgttcacgtagtggagcccttctcttggcacttcttctgcaagcaagcatggaagtcagaggttggtgtttggagagttcccaatgccaagacctgactactgagtcaaacctgctggaatgcataagagcctgtaaacccaaggaacaggtgaaacgctatggtgggtttctcaggaaatatcccaaaagatcctctgaggttgctggtgaaggtgatggggacagtatgggccatgaggacctgtacaagagatatggtggattcctcagaagaatcagacccaaactgaagtgggacaaccagaagagatatggagggtttctgaggagacagttcaaggtagtcaccagatcccaagaggaccctaacgcgtatagtggggaactctttgatgcatgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagaagcttacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa